SEQUENCE LISTING (13 sequences)

SEQ ID NO: 1 - DNA, 550 bp, Pseudomonas fluorescens
gcgcctgacgaagcggcgtagccacaaggagaaccgctgatgcgcctgtccgagctgaaaaaggccggccgcaccctgagcctgcccctgaccctcgatcttgcggacgccgtcggccccggccagttgcaactgctgagcctgttgcgtgtattgccgggcgagcgttacgtgggcgcggcggtctggcgcgggcgtgcggtgctggccaagttattggtgggcagcaaggccgcgcggcattttcagcgtgaactcacgggcgtgcgcctgctggccgaacacggcctgaccaccccccggttgctcgccgatggcttgcaggaaggcgagggcggttggttgctgttcgagttcctcgaaggcgccgaaagcctggccgatgcctggcaggcccgttcgaagcgctgccgccgctggccgacgaacaaaccgcggtgctcgccgaagcgctgggtgcgatcgcgcagatgcacaccaaggctgtggcagaagactgcatctggacacctgctgcgccaggacggcaagctgtacttgatcgacgtgc

SEQ ID NO: 2 - DNA, 5178 bp, Pseudomonas fluorescens
cgaattccttgatcttgtggatcttgccgttttcgccgtagccgaagaacgtggtggcgtactcacccttaccgtcgataccgaggatcgcggttttttcctggaagcccgagcagtggtaagcactggaggcgtgggccaggtggtgctcaaccggttcgatcttgattttcttcggatcgaagcccagttgctccaggcaccagacgatcttgttgcggtagcgcttgtagcgacggttgcccatcaggatcgcgtcgagggcacggtccggggcataccagtaacgcttggcgtagtgccagcgcgcctcgccgaacagactgatcggagcgaacgggatcgccaccacgtcaacgtcggaaggcttgatgccggcctgttccaggcagaacttcgccgactcgtagggcatgcggttctttgcatgtttgtcgcgtacgaagcgctcttcttcggcggccgcgatcagcttgccgtcgatatacagggctgcggaaggatcatggctaagggcgccggacaggccaagaatcgtcaatgccacaggggtctagcctcttttagtctgcatgcaggcgggtcgcgcctgaaaaaagtgtgcttcccgcctgggcaggaaacagctaaagggcgggattatagcttaaaagcagacgaaagctgttagcggcaagcggcaagccattaggaactagcggagttcaaggctgccgtgggcactttgccggtagatcgtataggggagaatgagcatgtcgaggacgccggagacaggcaagtcaagaacggcccagggcacgccgggaatgaatccggtaatacgatcgttcccacgcgcgcgtgggaacgatcaatacaggcctctcacgtgaagcacggggaaccatcacacttgcctgggcaaccgctgatcaatcagttggtacaacgcactgtcctgcggccagttacgcatgaaccgcgcccgatccctggcatatgccggtgcgaaactggcggctgagctgtgctgacagaccgaatccagatcaatcagcgcccagcggtcttcgtgccagaacaggttgtggcccttgaaatccccatggctgatgcgctcgccaataagttgggcaaacaggtggtccagggccagcaattcgttttccggcgcgtcaccgttgtcgatgtagggcgcaaagcgctcgatgatgtccgggcccggcaggtattcggtgatcaggtaggcacggctgcgcagccagaaaacgcgcttttccaatacggccagtggcttgggcgtggcgatacccaggaacgccaggcggttgccttcacgccaggagtgccaggcgcggcttgggcgccagaagcgtttgagccaatgggcgaaccccttgatgttgtaacgcttgatcaccaacggacgaccggccacctcgaccttggccacgctggcggcaccgccggtcttgtacaggtggccgcggtcgagcagcgcatcggcctgttccagcaccggcagcatcgcaggctcttcttcgcgacgaatcgcgcgcaaaccaaacgcaccgcgcactacgctgaacagcgtgcattcacggccggccttgttcaaaaagtccttcaagcgccaggcactgaccttgcgcacctgtttttccagcgcttgcagcggcaacgcgtgctcactgttgccgagcaggtaatacaccagcagctcctcggtaaacggctcgaggtttttcggcaattgggcgaaaaacacaccgaggttttccagtactcggttgcgcgacagcggtttaccggcctcttcgacacggatgcccgcaccgtcgatcaagtacagcttgccgtcctggcgcagcaggttgtccagatgcaggtcttcctgccacagccctttggtgtgcatctgcgcgatcgcacccagcgcttcggcgagcaccgcggtttgttcgtcggccagcggcggcagcgcttcgacggcctgccaggcatcggccaggctttcggcgccttcgaggaactcgaacagcaaccaaccgccctcgccttcctgcaagccatcggcgagcaaccggggggtggtcaggccgtgttcggccagcaggcgcacgcccgtgagttcacgctgaaaatgccgcgcggccttgctgcccaccaataacttggccagcaccgcacgcccgcgccagaccgccgcgcccacgtaacgctcgcccggcaatacacgcaacaggctcagcagttgcaactggccggggccgacggcgtccgcaagatcgagggtcaggggcaggctcagggtgcggccggcctttttcagctcggacaggcgcatcagcggttctccttgtggctacgccgcttcgtcaggcgctgcagccaggtggcgaccagcggcactgtcttcaggctggtcgagataagccgccagcaattggcgcacctgcgcgttcgaccactgcggtgcgcggcgcaacagcggctccaggtccttgacccggtcacgccagccgaacagcaacgggcgggttttctccaggtcgatcaactgcgcggcgtaaccgtcaccgggggcctgcaaaaaaatatgcttggggtaaaaacagccatgcacctggcccacgccatgcagccggcgcgccagccggccacaggccagcaggatggcccggtgttgggcgtcgctcagttgcggccattgctccagcaccgaatccaggtcgttccagccatccagggcacgggtcagcaacatcgcgcggtgttcgccagccaccttgcgttcgccatagaacgccgcctgcaacgccgggatgcccagcgtgcgataacggctgatattgcgaaactcgcgggagaaactcggctcgcccaacggccggtgcagactgcgggtgaggtagttgctctggcgcttgaggtaatacccgtggccctctatctccaggcggaacacactgctccaaccgccgcgactggtgttcggctcgtcgacggcgtccagttgcttggcccacagcgcgtcgaatgtcgccaggccgttacgctccagcacggcacggtcttcagccgccagaaaatcactcattcgcgcccctcgaaaaacttcaccacgtggcgaatccgccgtttgtccgaatcactcaggtgccgactgccacggtactgcaagtagaaacgcaggcgttgggtggccgacaggtgatacttggccactttgtccaggcaagccaggtccttggtgatgcggtatttgagccagaaaccacgccagaaatcgccgttgggacagtcgatcaaatacagggtcgactgatcatcgaccagcaggttgcgccacttcaaatcgttatgggtgaagcgatgatcgtgcatggtgcgcgtgtattcggcgagctggcggctgaccgcatcgacccatttggggtcgcgcaggcgcgcatcgcggtgctcggccagcaccgagaggtcttcggtgcgtggcaactcgcgggtgatcatcgcgccacggtcataggccaggccgttacgctccaggccccaggccaccacgtcggcggtgggtatgccccacttggcgaagcgcttgaggttctgccattcggacttgacccgcggcttgcccaggtagcgtcgcaggcccttgccggcgccgacgtagcgcttgacgtagtaattgaccccaccgcgctgcacccggatcacctccgacaacgggtcgcgggtcaggcgctcgccttgcagggcgaacaccgcttcgaggctgccgaaaatcatccgccaggttggcgtacgcaggttccaggatccaacccgccatcagagcgcatctccatatcgctgcttgcgtgcgtagagcttgtcggccttggcttgcagccactgcaacaacgcgctttcttcggccaggacctggcgcaacggctgctggaagtagcccttgagaaagcgcagcttgtcacggcgggtcaggccgatgtccagcgccgagaagtacagcgcggccaggtccttgtcgcgccagcgatgactgatcttgtcgcgggtctgggcgcggtgcaggtcgatcaccgaaagtttgaagtggtcggcggtcaccggcgtgtcagtgtgcaacaggaaatggcagatgtagcagtcgcgatggttgaccccgccgcgatgcatcatgcccgtcatgcgcgccacttcggcgatcagcgcacgcttgagacgtggctcgggcggctgtttgcgccagtcgatgctgaaatcttccaggctgacggtgggcgccaactcctcggtgacgatgaacgaatgctgatcggccgggttgctgccgcgctcgccataagccaccgcggtcatggtcggcacacccaatgcctgcaagcgctggatcgccagccattcctggccggcgcccaataccgggagtttggcggtgaccaggttcttgacgatctcgccccagccgatgccacggtgaatcttcacgaaatagccacggccatccacctcggtacgcaaggtgcgacgcgcttcgagttcacggtacacctcgccytccagcgccttcgacggcggcgaacgcatcgcgcccggcccagagggtcttgaacggttcggcaagcatcaacttcatggttttggctccgccagaatcacatccgcagcgtgctgcggcatgctgtagaggtcggccgtctcggcgaaggccaacccattgcggctccaggccgcgcgctgtgcagcgtcactcaacatacgtaccaggtagccgttgagctgcgcttgttcaaacggctcatccagcaccaggccgctgtcggcctcattgatgtaatgggcataaccacacaccgccgagaccagcaccggcaggccggccaccagggcttcgagcaacacggtgccggtattttcgttgtacgccgggtggatcaacaggtcggcgcccagcaggaaacgcggaatgtcgctgcgtcccttgaggaactgcacgttatcgcccaagcccaacgtggcgctctgcaattggaatactttggggtcgtcctggccgattacaaacagacgggtgcgtttcttcagttccgcgggcaaggcggccacagccttgaggctgcggtcgacgcccttggtcttgaagcccgagccgatttgcaccagcaacaggtcgtcatcgccgaggttgaattc

SEQ ID NO: 3 - protein, 459 aa, Pseudomonas fluorescens
MRLSELKKAGRTLSLPLTLDLADAVGPGQLQLLSLLRVLPGERYVGAAVWRGRAVLAKLLVGSKAARHFQRELTGVRLLAEHGLTTPRLLADGLQEGEGGWLLFEFLEGAESLADAWQAVEALPPLADEQTAVLAEALGAIAQMHTKGLWQEDLHLDNLLRQDGKLYLIDGAGIRVEEAGKPLSRNRVLENLGVFFAQLPKNLEPFTEELLVYYLLGNSEHALPLQALEKQVRKVSAWRLKDFLNKAGRECTLFSVVRGAFGLRAIRREEEPAMLPVLEQADALLDRGHLYKTGGAASVAKVEVAGRPLVIKRYNIKGFAHWLKRFWRPSRAWHSWREGNRLAFLGIATPKPLAVLEKRVFWLRSRAYLITEYLPGPDIIERFAPYIDNGDAPENELLALDHLFAQLIGERISHGDFKGHNLFWHEDRWALIDLDSVCQHSSAASFAPAYARDRARFQV

SEQ ID NO: 4 - protein, 351 aa, Pseudomonas fluorescens
MADALANKLGKQVVQGQQFVFRRVTVVDVGRKALDDVRARQVFGDQVGTAAQPENALFQYGQWLGRGDTQERQAVAFTPGVPGAAWAPEAFEPMGEPDVVTLDHQRTTGHLDLGHAGGTAGLVQVAAVEQRIGLFQHRQHRRLFFATNRAQTKRTAHYAEQRAFTAGLVQKVLQAPGDLAHLFFQRLQRQRVLTVAEQVIHQQLLGKRLEVFRQLGKHTEVFQYSVARQRFTGLFTDARTVDQVQLAVLAQQVVQMQVFLPQPFGVHLRDRTQRFGEHRGLFVGQRRQRFDGLPGIGQAFGAFEELEQQPTALAFLQAIGEQPGGGQAVFGQQAHAREFTLKMPRGLAAHQ

SEQ ID NO: 5 - DNA, 2391 bp, Pseudomonas fluorescens
tcgctggtcgccacctggctgcagcgcctgacgaagcggcgtagccacaaggagaaccgctgatgcgcctgtccgagctgaaaaaggccggccgcaccctgagcctgcccctgaccctcgatcttgcggacgccgtcggccccggccagttgcaactgctgagcctgttgcgtgtattgccgggcgagcgttacgtgggcgcggcggtctggcgcgggcgtgcggtgctggccaagttattggtgggcagcaaggccgcgcggcattttcagcgtgaactcacgggcgtgcgcctgctggccgaacacggcctgaccaccccccggttgctcgccgatggcttgcaggaaggcgagggcggttggttgctgttcgagttcctcgaaggcgccgaaagcctggccgatgcctggcaggccgtcgaagcgctgccgccgctggccgacgaacaaaccgcggtgctcgccgaagcgctgggtgcgatcgcgcagatgcacaccaaagggctgtggcaggaagacctgcatctggacaacctgctgcgccaggacggcaagctgtacttgatcgacggtgcgggcatccgtgtcgaagaggccggtaaaccgctgtcgcgcaaccgagtactggaaaacctcggtgtgtttttcgcccaattgccgaaaaacctcgagccgtttaccgaggagctgctggtgtattacctgctcggcaacagtgagcacgcgttgccgctgcaagcgctggaaaaacaggtgcgcaaggtcagtgcctggcgcttgaaggactttttgaacaaggccggccgtgaatgcacgctgttcagcgtagtgcgcggtgcgtttggtttgcgcgcgattcgtcgcgaagaagagcctgcgatgctgccggtgctggaacaggccgatgcgctgctcgaccgcggccacctgtacaagaccggcggtgccgccagcgtggccaaggtcgaggtggccggtcgtccgttggtgatcaagcgttacaacatcaaggggttcgcccattggctcaaacgcttctggcgcccaagccgcgcctggcactcctggcgtgaaggcaaccgcctggcgttcctgggtatcgccacgcccaagccactggccgtattggaaaagcgcgttttctggctgcgcagccgtgcctacctgatcaccgaatacctgccgggcccggacatcatcgagcgctttgcgccctacatcgacaacggtgacgcgccggaaaacgaattgctggccctggaccacctgtttgcccaacttattggcgagcgcatcagccatggggatttcaagggccacaacctgttctggcacgaagaccgctgggcgctgattgatctggattcggtctgtcagcacagctcagccgccagtttcgcaccggcatatgccagggatcgggcgcggttcatgcgtaactggccgcaggacagtgcgttgtaccaactgattgatcagcggttgcccaggcaagtgtgatggttccccgtgcttcacgtgagaggcctgtattgatcgttcccacgcgcgcgtgggaacgatcgtattaccggattcattcccggcgtgccctgggccgttcttgacttgcctgtctccggcgtcctcgacatgctcattctcccctatacgatctaccggcaaagtgcccacggcagccttgaactccgctagttcctaatggcttgccgcttgccgctaacagctttcgtctgcttttaagctataatcccgccctttagctgtttcctgcccaggcgggaagcacacttttttcaggcgcgacccgcctgcatgcagactaaaagaggctagacccctgtggcattgacgattcttggcctgtccggcgcccttagccatgatccttccgcagccctgtatatcgacggcaagctgatcgcggccgccgaagaagagcgcttcgtacgcgacaaacatgcaaagaaccgcatgccctacgagtcggcgaagttctgcctggaacaggccggcatcaagccttccgacgttgacgtggtggcgatcccgttcgctccgatcagtctgttcggcgaggcgcgctggcactacgccaagcgttactggtatgccccggaccgtgccctcgacgcgatcctgatgggcaaccgtcgctacaagcgctaccgcaacaagatcgtctggtgcctggagcaactgggcttcgatccgaagaaaatcaagatcgaaccggttgagcaccacctggcccacgcctccagtgcttaccactgctcgggcttccaggaaaaaaccgcgatcctcggtatcgacggtaagggtgagtacgccaccacgttcttcggctacggcgaaaacggcaagatccacaagatcaaggaattcg

SEQ ID NO: 6 - DNA, 520 bp, Pseudomonas fluorescens
  misc_feature (444)..(444): “n” equals any nucleotide.
ataggcacctacgtgcaggcgcaacgatgaagccgtctcggcgggtgcgacgttgaaacgcgccacattgatgatctgcccgctgtccaccgaaggggccaggtgatggcaggtcgagccgtaggtttcatcgttgtagtagatcgcgtaatgctggctgcccaggccccggtacttgggcggtgccgggtgcagattgatcgcgcctttgcgggcattcttgtagatgctcggcggaaaaatgaagtcgccacggtaggagatgatccaatcgccttcccagttatccagatggtaaggatagggatcgcccgggtcccagcaaaacacctcaaggttggagaatacggtcttggcgaattcacgccgtgatcgcaccagtccatggtgcacaccgacagcaggaccttgtcttgcatggctcgattgtctgcatcttacttnttcatttcaggtaatgtgggtgacaacgcccgccccccgtcaaagcccgccttgtcgctagtacgcgacagcctta

SEQ ID NO: 7 - DNA, 5040 bp, Pseudomonas fluorescens
  misc_feature (691)..(691): “n” equals any nucleotide.
ggatcccgaagttcgctacttgcccaacggtaacgccgtgaccaacctgagtctggcgaccagcgaacagtggaccgacaagcagaccggccagaaggtcgagaagaccgaatggcaccgtgtgtcgatgttcggcaaggttgcagagatcgctggtgagtacctgcgcaaaggttcgcaggtctacatcgaaggcaaactgcaaacccgcgagtgggaaaaagacggcatcaagcgttacaccaccgagatcgtggtcgacatgcaaggcaccatgcaactgctgggcggccgtccacagggcgaccaacaggggcagggtggcatgtccaactcggcaccgcgtccacaacagtcgcgtccacagccaagccagcagccacagcgtgagtcgcgtccagcgccccagcaggccgctgcgcaaccggcgccggatttcgacagctttgatgacgatattccgttctaacgcagctttcagtaccgtaacccataaaaaagcgaagccattaatctggcttcgctttttttatggccacagatttttgtgaagtaacaactgcgtgcctgggatcaactcgtatgctgcagcagcggatacagcgaaatcaccagcaacgccgccatgccgaaattgaacatgcgcagccagcgcgggttgcgcagcacgttgcgcagtacactgccgaancccgcccatacccccacgcttggggcgttgatcagagcgaacaccgtagcgatgatcaccacgttcatggtgtagccctgcaagggcgtgtacgtgctgatcgcaccaattgccatgacccacgccttgggattgacccactgaaacgccgctgcgccccagaaccccaggggcttgcctttaccttcggtgtcttctgatgccgggccggaacgggcgatgttccatgccaggtacagcaagtaagccgcgcccgcatagcgcagcacggtgtagagcaccggataggcggtaaaggctgcacccagcccaagccccacggccaataccagcaccaggaacccggagctgatacccaggatgtgcgggatggtgcgctggaagccaaagttgacgcccgaggccaacagcatggcgttattggggccaggggtgacggaagtgacaaacgcaaacaaagcaaacgccagcattaaatcaaacgacaaggacatggatatttcctggaaacgggccttcgcgcttacctgccacccggacagcgcgggatacaggtgcagtgtggccacttgtagttggagtcagaacaacgaactgagaaaaccaatggcaaccatcgagtagaacgctgcacaaatcagcccgaaggacttgcgcgtccgggtgaaaaacggttctgccgaatgggcggaaaacgctaaggcgtaggtacagaaaatcaggaaacccaatagcgcacaggccgccaccagcacgaaactggcccaggccggcgcgttgacgctcaggcccacggtggtcaccgtgaaccaggtcaaggccgccttggggttagtcaggtgaatgcccaacccctgcaaatagaagcccaggttgcgcgtctgcttttcccgtactttggctaccgtcgtggcgttgtgcgccagcgcactgcgcaccgatttccaggccaggaaaaacagatagcaggcgccgaacacttttaaccacaggatgatctgggtgttggacatcaacaacgccgacacgcctgcggcggtcatcgcgccccagcacaacgacccggaaatcaccccggccgccaacgccaggcccggtgtgcgcccgtagttcaaggaggtgttggcgatggccaggttgcccgggccagggctggcggtgccgatcacgtagacccccagtgccgctgcaaaactcgaaaactcaaacatagatgcactcatacccgtcgcgccagcgcgcagtgagcacttcaagccacccatggcttgaagtgacggccaagaaaaagctatttgaagcaacgatgatccggctcctgggcgcggatcttctccatccagggcttgagttccgattgcttgtacaggcgctcgccccaattttccggcgagaccggcagtggccgtcccagcaggatgtagtcggtgagcaggtggatgaactgctgcaggcaataggcacctacgtgcaggcgcaacgatgaagccgtctcggcgggtgcgacgttgaaacgcgccacattgatgatctgcccgctgtccaccgaaggggccaggtgatggcaggtcgagccgtaggtttcatcgttgtagtagatcgcgtaatgctggctgcccaggccccggtacttgggcggtgccgggtgcagattgatcgcgcctttgcgggcattcttgtagatgctcggcggaaaaatgaagtcgccacggtaggagatgatccaatcgccttcccagttatccagatggtaaggatagggatcgcccgggtcccagcaaaacacctcaaggttggagaatacggtcttggcgaattccacgccgtgatcgcaccagtccatggtgcacaccgacagcaggaccttgtccttgcatggctcgattgtctgcatcttcacttctccatttccaggtaatgtgggtgacaacgcccgcccccccgtcaagcccgcgctgtcgctagtacgcgacagccctgaccactgcgttgaagcgttcgaccatttgccccagctccccttcgctggtgataaacggcggtgcaaacagcacatgattgccgtgcgttccctgcacggtgccgctgccgggatagatcaacaggccgcgctgcaacgcctcttgcttgagcgccgcggcatacgcgccgccgcccttgaacggcgccttggtggcgcggctctcgacaaactcgacgcccacgaacagcccgcgcccgcgcacatcaccgacgatatccaggtcagccaggctttccctgagccaggcgcgcaattgctcaccgcgttggcgcacttgcgccagcaggtcttcgtcctcgatgacttgttgcacttccaaggcaatggcacaggccagcgggatggttgacgtgggtctggccgttgcccagcacgcctgaaccacgggccattaccgagtgcacctggtcgctgatcaacagtgccgagatcggcatgtagcccgccgccaggcctttgccgaccgcgaccatgtccggcacgatgccgtcgtcggcataggcgaacagctgcccggtacgccccatgccggccatcacctcatcgaggatcagcagcacatcgtgacgctcgcacacggccttgatcttgcgaaagtaaccgggcaccgcaggcaccgcgccattggtggagcccaccacggtttcggcaaagaacgccgcgacgttttcactgcccaggctgcgaattttggcgtccagttcatcggccaggcgcgtggcgtactgctcttcgctttcgtcgggccgttgatcacggtaggcgtagcatggcgagacaaactcggccgccgcgaacagttgaccaaacaccgattggcgctgcgggttaccggagatgctcaaggtgcccagggtgctgccgtggtagctctgccggcgggaaatgaacaacgacttttcaggcaagccgcgttcgcattggtactggtaagcgattttcatcgccagctccatcacctccgagccgccggataaaaactgcgcacgcgccaggccgccactggctgccaccaggcgttcggccagttcctcggcggcggcggtggtgaagctgcccgcgtgcgcccacgccagtttcttcgcttcgcgctcgaatgccgctgcgatacgcggatggccatggcccaggctcgacaccgcgacaccgccgcaggtgtcgaggtagcgtttgcccgttgcatcctcaagccacacccctgcgccgccgaccgccaggatcggcgaggtgttgagatttttgtaaatcacttttgacatgcgcgcggttccttgggcgattggccttacgggttacttacggagtgatcccaagccgtcggcgcctggtgaaagcgccgcccgaaacgggacgaatgaaacaccgaacgcgtcagcccgatgggcagcaacacaacgataaacatcaaaaacgcaagaaatcttcatgcagcggtaaaagctccaaagagtgaaagggggtggggatgcagtcccagccgggatgctacgctgcgtccagtcaacggcctcgacgaaggccggctgggcggggcgctcaagcacgtagcggcccagaaccagcacgtccatttccgtgcgcatgaagcaggcgtacgcttcctgcggcgtgcacacgatcggttcgccacgcacgttgaacgaggtgttgaccaacaccgagcagccggtgtgctccttgaaactgcgcagcaggtaggtgaacggtgcgttgttttcttcggtcaccgtttgcacccgcgccgacaggtcgacgtgggtcaccgccggcaactgcgagcgaatatggttgatgctgcccaagccccgctcggggccgccttcttgcagcgactgctgggatttgatcgagtcggtgaccggcgcgaccatcaacatgtacgggcttttttcgcagatatcgaaatagttgccggcgtcttcggccagcacggccggggcgaacgggcggaacgactcgcggtatttgatcttcaggttcatggtgcgctgcatctcggggttgcgcgcatcgccgagaatcgaacgcgcgcccaaggcccgtgggccaaactccatgcgcccctggaaccaacccaccaccgcaccgtcggccaggcgactggcgacctggtcatacagcccggcgtcgtcgtagcgggtaaaggggtagtggttttccagcaggaactgggcgatttcttcatcgccaaaacccggccccagcaggctgccgctcatggcatcggacttgttgccatccaagtgcgggcgaccgctgtgcttgaccgcaaaatccaacgccgcccccagggcacaaccggcatcgccggccgccggctggatcc

SEQ ID NO: 8 - protein, 200 aa, Pseudomonas fluorescens
MQTIEPCKDKVLLSVCTMDWCDHGVEFAKTVFSNLEVFCWDPGDPYPYHLDNWEGDWIISYRGDFIFPPSIYKNARKGAINLHPAPPKYRGLGSQHYAIYYNDETYGSTCHHLAPSVDSGQIINVARFNVAPAETASSLRLHVGAYCLQQFIHLLTDYILLGRPLPVSPENWGERLYKQSELKPWMEKIRAQEPDHRCFK

SEQ ID NO: 9 - DNA, 549 bp, Pseudomonas fluorescens
gcccaggacagcggcaccatgcaggcctggctcgaccgctatttcaaccaggcccatcgcctgccggatttgagcccatcggggttcaagccggtcagcgggcgtttgctcagcaccgagcaaggcgctgccgccatggtgctttaccaggacgcgcaaggccggcgcatcagtttctatatccggccgccggggccgaacaacggttttctaccgcgtggcagccgcaccgcagatgggctgcaagcgcaatactggtccggcggcggctacaactatgcggtcgtcagcccggcggaccaggtgcccgcacacctgttgcagttctaggcgcggcgctcctgtattggtcaaatcagcgcgagcgccctaaagtgagcgaccaccgcactacagagtgactcgcattggccgtgcttccctgctccgtcgttactcggtaaacacagcgccccacgacacctcgccatcaggcattctttgagaaccaggcccggcaacaggttacacctcagcgcgcagtccggcatcgccgcatgacgcgaaccg

SEQ ID NO: 10 - DNA, 3279 bp, Pseudomonas fluorescens
ctgcaggcgttcgatcaactcaacaccgaacaacgcgcgttgttgctgtgggtctcggtcgaaggcttgagctacaaggaagtcgccgaaatactcgacgtgccgcttggcaccgtgatgtcacgcctgtcccgcgcccgccaagccctgcggcaactcagcgacggcgaaattgccagcccttccctgcggatactcaaatgatcagcctgccccccagcgaacgcgacctgcatgcctacgtcgatcaccaactcctggagagcgatcgccgtgttctcgaaacgtggctgaccgcgcaccccgacgtcgcggcccaagtgcatgcctggcagcaggatgcgcagttgctgcgcgcgtcattgagcggcgccctgcaacagcccgccaaccccaaccttgacccggcgctgattcgccagcgcatccagcaccggtcgcgccgccacttcgccacggccgccgtgttgctgatcgccgtgagccttggcggcctcggcggctggcatgcccgtgaagccacgcaatcaccccaacagccaatggccgacgcgatgcaagcgttccggctgtttgcccaggacggcatcctgcccgccgattacaacgcccaggacagcggcaccatgcaggcctggctcgaccgctatttcaaccaggcccatcgcctgccggatttgagcccatcggggttcaagccggtcagcgggcgtttgctcagcaccgagcaaggcgctgccgccatggtgctttaccaggacgcgcaaggccggcgcatcagtttctatatccggccgccggggccgaacaacggttttctaccgcgtggcagccgcaccgcagatgggctgcaagcgcaatactggtccggcggcggctacaactatgcggtcgtcagcccggcggaccaggtgcccgcacacctgttgcagttctaggcgcgggcgctcctgtattggtcaaatcagcgcgagcgccctaaagtgagcgaccaccgcactacagagtgactcgcattggccgtgcttcccctgctccgtcgtttactcggtaaacacagcgccccccacgacacctcgcccatccaggcattctttgagaaccaggcccggcaacagggttacaccctcagcgccggccaggtccgggcaatcgccgccatggcacgcgaaacccggcaccggctcgcccgccggcccactcgaagcctgtacctgcacggtccggtgggacgtggcaaaagctggctgctggacggtttcttccaggcgctgcccatcgccgagaaacagcgtgtgcatttccatgagtttttcgcgcgcttgcaccgcggcatgttcgcccaccgggccgtggcacgatgcgctcgccatgaccctcgatgagttgctcgacggctgccaggtgctgtgtttcgacgagttccacgtccacgacatcggcgacgccatgctgatcacgcggctgttccaggccctgttccggcgcggtgtctgggtgctggtcacctccaactatccgcctgaaggcctgttgcccaaccctctgtaccacgagcgtttcaagccggtgatcgacttgatcgccgcgcgcatggacgtcttggaagtcagctcccccgaggattttcgccgcttgccccaggcccacgccacgcaacgctttaccacggggcagtatgtgtggcccggcaccgcggcccaacgcgcgacgctcggcctccccgccacggatggcccggcgcataccctggcagtgggcaaccggcagttggcctgccgctaccatcaggcgcgaacggtcgcgttcaccttcagcgacctctgcgaacaactgacggccgtgatggattacctgctgctgtgcgaagatttcgaccactggatcatcgacggcctgcctcacctggccgagtgtccgattgccgtgcagcagcgctttatcaacctggtggacgtgctctacgacaaagacaagcacctggtgctgatcggtgaacagccactggcacaggcgatgagtggcgaggccatcgacctcgcccgcaccgccagccgcctgaaccaattgcaacaggccagcccgcaaccggtgcccgacccggtatcatgagcgccctttacgccccctagccgagtgaccgcgccgctgatgcataccctcgcccaactcaagtccggccaattggccggtatccagcgcctggacctgtcctgcgggctcaccgaatttccccgggaaatcttcgaactggccgactccctggagatcctcaacctcagcggtaacgccctgagccggttgcccgacgacctgcatcgcctgccacacctgcgggtactgttctgctcggacaacgccttcaccgaactgcccgaatgcctgggccagtgcgcgcaactgagcatgatcggtttcaaggccaaccagatcagccacgtgcccgccgccgcgctgccgccgttgctgcgctggctgatcctcaccgacaaccgtatcagccaattgcccgatgaattgggcgagcgcccactgctgcaaaaactgatgctggccggcaaccaattggcgcatctgccgccgagcctgagtcactgtcaccacctcgaattgctgcgcatcgcgtccaaccgcttcacccagctgccggcgtggctgttgaccctgcccagcctgacctggctggcctatgccggtaacccggtggaaatggcggtggatgtagcggtggacgacgccacgcccgacattccttgggccgagctggaactggcccaagtgctgggcgaaggcgcttcgggggtgattcgcaaagcgctgcgtaaacccacgggcacgcccgttgccgtcaagctctacaaaggcaccatcaccagcgacggttcgccgttgcacgaaatgcaagcctgcatcgccgccgggctgcaccccaacctgatcagggtgcagggccgcgtcatcggccaccccgatgaccaggctgcgctggtgatggacctgatcgacccgagctaccgcaacctggcggccctgccgagcctggcctcgtgtacccgtgacatctacgcgcccgacacgcgtttcagcgccaaagtggccttgcgtatggcacggggcatcgcctcggtggccgcgcatctgcaccggcacggcattacccatggcgacctctacggccacaacatcctgtggaatgaagccggggattgcctgctcggggattttggcgcggcgtcgttccatgccacggcggataccctcgaaacacgggcgctgcag

SEQ ID NO: 11 - protein, 67 aa, Pseudomonas fluorescens
LQAFDQLNTEQRALLLWVSVEGLSYKEVAEILDVPLGTVMSRLSRARQALRQLSDGEIASPSLRILK

SEQ ID NO: 12 - protein, 230 aa, Pseudomonas fluorescens
MISLPPSERDLHAYVDHQLLESDRRVLETWLTAHPDVAAQVHAWQQDAQLLRASLSGALQQPANPNLDPALFATAAVLLIAVSLGGLGGWHAREATQSPQQPMADAMQAFRLFAQDGILPADYNAQDSGTMQAWLDRYFNQAHRLPDLSPSGFKPVSGRLLSTEQGAAAMVLYQDAQGRRISFYIRPPGPNNGFLPRGSRTADGLQAQYWSGGGYNYAVVSPADQVPAHA

SEQ ID NO: 13 - protein, 14 aa, Pseudomonas fluorescens
MVERFNAVVRAVAY

(19) United States
(12) Patent Application Publication
(10) Pub. No.: US 2006/0147438 A1
(43) Pub. Date: Jul. 6, 2006

(54) Bacterial bioherbicide for control of grassy weeds

(76) Inventors: Mark Azevedo, Albany, OR (US); Dallice I. Mills, Corvallis, OR (US); Aleta M. Groenig, Hillsboro, OR (US); Brian W. Russell, Bend, OR (US); Donald Armstrong, Corvallis, OR (US); Gary Banowetz, Corvallis, OR (US); Lloyd Elliott, Corvallis, OR (US)

Correspondence Address: KLARQUIST SPARKMAN, LLP, 121 SW SALMON STREET, SUITE 1600, PORTLAND, OR, 97204, US

(21) Appl. No.: 10/537,017

(22) PCT Filed: Dec. 5, 2003
(86) PCT No.: PCT/US03/38653

§ 371 Date: Jun. 1, 2005

Related U.S. Application Data
(60) Provisional application No. 60/431,651, filed on Dec. 6, 2002.

(51) Int. Cl. A01N 63/00; C12N 1/20

(52) U.S. Cl. 424/93.47; 435/252.34

(57) ABSTRACT

The present disclosure concerns specific Pseudomonas fluorescens and Pseudomonas putida strains, specific genes and their corresponding specific gene products, the specific bioherbicide molecules whose synthesis or secretion is controlled by those genes and gene products, and methods of using these molecules to control deleterious weeds.

### CROSS-REFERENCE TO RELATED APPLICATION

This claims the benefit of U.S. Provisional Application No. 60/431,651, filed on Dec. 6, 2003, which is incorporated herein by reference in its entirety.

### GOVERNMENT RIGHTS

The claimed invention was developed, at least in part with United States government support under USDA CSREES Grant No. 00-34321-9798. The United States government has certain rights in the invention.

### FIELD

The present disclosure concerns specific Pseudomonas fluorescens and Pseudomonas putida strains, specific genes and their corresponding specific gene products, the specific bioherbicide molecules whose synthesis or secretion is controlled by those genes and gene products, and methods of using these molecules to control deleterious weeds.

### BACKGROUND

Annual bluegrass (Poa annua), roughstalk bluegrass (Poa trivialis) and downy brome (Bromus tectorum) are serious weed problems in grass-seed production fields of the Pacific Northwest and other regions. Annual costs to remove P. annua from perennial ryegrass seed to comply with federal and state seed certification standards can exceed $30,000,000 for the Willamette Valley alone (Burr, Section 18, “Emergency Exemptions Requests for Use During the 1997-98 Field Season in Oregon Grass Seed Crops,” Oregon Seed Council, Ag. Research, Inc. 1998). Grass seed producers in Central and Eastern Oregon, as well as regions of Washington and Idaho, face similar difficulties. Increased weed seed contamination in Oregon and Washington also result from state-mandated reductions of open field burning to improve air quality.

Concurrently, controls on chemical herbicides registered for use against P. annua and other weedy grasses have become more stringent. Increased dependence on a limited number of chemicals has been accompanied by the emergence of herbicide-resistant biotypes of P. annua and B. tectorum (Gamroth, “Resistance of annual bluegrass (Poa annua L.) to diuron and ethofumesate,” M. S. Thesis, Oregon State University, Corvallis, 1997; Mueller-Warrant, “Herbicide Resistant Weeds, Prevention and Contro,” 5th Grass Seed Cropping Systems for Sustainable Agriculture Review, 1998). P. annua biotypes that are resistant to herbicides also have been observed in other parts of the nation (Kelly and Coats, Proceedings, Southern Weed Science Society, 51:90, 1998a; Kelly and Coats, Proceedings, Southern Weed Science Society, 51:71, 1998b).

During the past decade, the number of registered chemical herbicides has decreased because of their adverse effects on the environment. Because of the relatively broad specificity of these compounds and their ability to persist in the environment they may impose significant environmental risks. Recognition by the general public and environmental agencies of the need to develop environmentally friendly control strategies has placed the continued use of some chemicals in jeopardy. Moreover, annual bluegrass biotypes that are resistant to synthetic chemical herbicides have reduced the efficacy of these chemicals for control of weeds.

An alternative to herbicides is the use of deleterious rhizobacteria to suppress or reduce growth of weed populations (Boyetchko, HortScience, 32(2):201-205, 1997). For example, pathovars of Xanthomonas campestris pv. poannua are systemic pathogens of P. annua that have been used for biocontrol of annual bluegrass. However, these strategies invariably employ a live organism as the biocontrol agent, which has proven problematic in practice. The effectiveness of live organisms can only be realized if the organism remains viable. Environmental conditions, including soil type, soil pH, temperature, moisture, and competition for nutrients from other microbes frequently preclude live organisms from being effective as biocontrol agents. Thus, the use of live organisms as biocontrol agents often has yielded irreproducible results and ineffective control when organisms are introduced into different soils and new environments. The introduction of live organisms also may result in environmental impact issues.

In view of the above considerations, there is a need for effective alternatives for the control of grassy weeds.

### SUMMARY OF THE DISCLOSURE

Described herein are specific Pseudomonas fluorescens and Pseudomonas putida strains, specific genes, specific gene products, the specific bioherbicide molecules whose synthesis and secretion are controlled by those genes and gene products, and methods of using these molecules to control deleterious weeds. One embodiment of the disclosure is bacterial strain, Pseudomonas fluorescens Biotype C WH6, wherein the bacterial strain inhibits or arrests grassy weed germination. Other embodiments of the disclosure are the bacterial strains Pseudomonas fluorescens Biotype C WH19, Pseudomonas fluorescens Biotype B E34C, Pseudomonas putida Biotype B AH4, and Pseudomonas putida Biotype B AD31. These bacterial strains also inhibit or arrest grassy weed germination.

Also disclosed herein is a Germination-Arrest Factor. The factor is produced by Pseudomonas fluorescens Biotype C WH6, Pseudomonas fluorescens Biotype C WH19, Pseudomonas fluorescens Biotype B E34, Pseudomonas putida Biotype B AH4, or Pseudomonas putida Biotype B AD31, and inhibits or arrests grassy weed germination.

A further embodiment of the disclosure is an isolated nucleic acid as set forth in: (a) SEQ ID NO: 2, (b) SEQ ID NO: 7, (c) SEQ ID NO: 10, or (d) sequences having at least 90% sequence identity to (a), (b), or (c); wherein the nucleic acids encode proteins involved in the synthesis or secretion of Germination-Arrest Factor (GAF).

Yet another embodiment of the disclosure is an isolated Germination-Arrest Factor protein comprising an amino acid sequence as set forth in: (a) SEQ ID NO: 3; (b) SEQ ID NO: 4; (c) SEQ ID NO: 8; (d) SEQ ID NO: 11; (e) SEQ ID NO: 12; (f) sequences having at least 90% sequence identity to (a), (b), (c), (d), or (e); or (g) conservative variants of (a), (b), (c), (d), or (e); wherein the Germination-Arrest Factor protein is involved in the synthesis or secretion of Germination Arrest Factor that inhibits or arrests germination in grassy weeds.

Also disclosed herein is a method of inhibiting or arresting weed germination. The method includes applying Pseudomonas fluorescens Biotype B B34, Pseudomonas fluorescens Biotype C WH19, Pseudomonas fluorescens C Biotype WH6, Pseudomonas putida Biotype B AH4, or Pseudomonas putida Biotype B AD31, or mixtures thereof; to a growth medium in which it would be desirable to inhibit or arrest grassy weed germination, thereby inhibiting or arresting grassy weed germination. Still further embodiments of the disclosure are methods of inhibiting or arresting weed germination that include applying the Germination-Arrest Factor to a growth medium in which it would be desirable to inhibit or arrest grassy weed germination, thereby inhibiting or arresting grassy weed germination.

A further embodiment of the disclosure is a method of using Germination-Arrest Factor as a seed-cleaning adjuvant in seed-cleaning processes as a supplement or alternative to physical removal of target weed seeds.

Further embodiments are compositions for inhibiting or arresting the germination of grassy weeds. The compositions of this disclosure include Germination-Arrest Factor and a timed-release coating coating the Germination-Arrest Factor.

Still another embodiment of the disclosure is a method of inhibiting or arresting weed germination in a grass patch The method includes broadcasting the Germination-Arrest Factor at least once a year across a grass field in which inhibiting or arresting weed germination is desirable, thereby inhibiting or arresting weed germination.

Yet another embodiment of the disclosure is a method of producing a Germination-Arrest Factor. The method includes culturing Pseudomonas fluorescens Biotype B E34, Pseudomonas fluorescens Biotype C WH19, Pseudomonas fluorescens C Biotype WH6, Pseudomonas putida Biotype B AH4, or Pseudomonas putida Biotype B AD31 in a suitable culture medium, collecting the culture medium, and purifying the culture medium to produce the Germination-Arrest Factor.

Also disclosed is a kit for inhibiting or arresting grassy weed growth. The kit includes a Germination-Arrest Factor, a container, and, optionally, instructions for using the kit.

Yet another embodiment of the disclosure is a method of using Germination-Arrest Factor to investigate regulation of seed germination and seedling development by using Germination-Arrest Factor to probe for regulatory sites in plant cells and to investigate regulatory mechanisms controlling seed germination and development.

Still other embodiments are Pseudomonas fluorescens and Pseudomonas putida bacterial strains having the GAF-producing characteristics of Pseudomonas fluorescens Biotype B E34 (deposited as NRRL # B-30481), Pseudomonas fluorescens Biotype C WH19 (deposited as NRRL # B-30484), Pseudomonas fluorescens C Biotype WH6 (deposited as NRRL # B-30485), Pseudomonas putida Biotype B AH4 (deposited as NRRL # B-30482), or Pseudomonas putida Biotype B AD31 (deposited as NRRL # B-30483).

### Sequence Listing

SEQ ID NO: 1 is the nucleotide sequence of Pseudomonasfluoresceis DNA flanking one side of the site of Tn5 insertion at the GAF1 locus.

SEQ ID NO: 2 is the nucleotide sequence of the 5.2 kb EcoRI fragment cloned from strain WH6.

SEQ ED NO: 3 is the amino acid sequence of the ORFc polypeptide from the EcoRI fragment.

SEQ ID NO: 4 is the amino acid sequence of the ORFe polypeptide from the EcoRI fragment.

SEQ ED NO: 5 is the nucleotide sequence of the terminal 2.4 kb EcoRI-PstI fragment cloned from strain WH6.

SEQ ID NO: 6 is the nucleotide sequence of Pseudomonas fluorescens DNA flanking one side of the site of Tn5 insertion in strain WH6-2.

SEQ ID NO: 7 is the nucleotide sequence of the 5 kb BamHI fragment cloned from strain WH6.

SEQ ID NO: 8 is the amino acid sequence of the ORFb polypeptide from the BamHI fragment

SEQ ID NO: 9 is the nucleotide sequence of Pseudomonas fluorescens DNA flanking one side of the site of Tn5 insertion in strain WH6-3.

SEQ ID NO: 10 is the nucleotide sequence of the 3.4 kb PstI fragment cloned from strain WH6.

SEQ ID NO: 11 is the amino acid sequence for the putative PrtI polypeptide encoded by strain WH6.

SEQ ID NO: 12 is the amino acid sequence for the putative PrtR polypeptide encoded by strain WH6.

SEQ ID NO: 13 is the amino acid sequence of the ORFc polypeptide from the BamHI fragment.

### BRIEF DESCRIPTION OF THE FIGURES

FIG. 1 is a digital image illustrating the various Germination Scores in the Standard GAF (Germination Arrest Factor) Bioassay System (see Example 1). Seeds shown are Poa annua seeds. The digital images shown illustrate the typical appearance of Poa annua seeds and seedlings following GAF treatments that generated the indicated Germination Scores. Germination Scores are shown in the upper right side of each digital image. E=embryo. P=plumule. C=coleorhiza. P.R=primary root L.S.=leaf sheath. F.L.=first leaf. A.R=adventitious root Bar=1.0 m

FIG. 2 is a pair of graphs showing the relationship between Germination Score and Pseudomonas fluorescens WH6 culture filtrate concentration (FIG. 2A), and the relationship between Germination Score and the concentration of GAF activity (FIG. 2B). Germination was measured in the Standard GAF Bioassay System using seeds of Poa annua. FIG. 2A-A series of dilutions of culture filtrate from Pseudomonas fluorescens WH6 was prepared and tested in the Standard GAF Bioassay System as described in Example 1, using seeds of Poa annua. Germination score was plotted as a function of culture filtrate concentration. FIG. 2B-GAF concentration was plotted as GAF Equivalents per Liter, where a GAF equivalent of 1.0 is defined as the minimal amount of GAF activity that will give a Germination Score of 1.0 when dissolved in one liter of solution.

FIG. 3 is a pair of digital images showing that treatment of Arabidopsis with a GAF extract prepared from WH6 culture filtrate (FIG. 3A) produced no effect on seed germination as compared to control seeds (FIG. 3B) germinated in the absence of GAF. FIG. 3A shows germination of GAF-exposed seeds, whereas FIG. 3B shows control seeds. Arabidopsis seeds were surface sterilized by exposure for two minutes to a 50% (v/v) bleach solution containing 5% (v/v) Tween-20 (as a surfactant). The seeds were then rinsed thoroughly with sterile distilled water, and sown onto the agar media described below. Germination of Arabidopsis seeds was evaluated at seven days after treatment. Bar=1 mm FIG. 3A-Arabidopsis seeds were sown onto sterile 2% (w/v) agar containing 10% (by volume) of a concentrated (20×) GAF extract prepared by extracting dried WH6 culture filtrate with 90% (v/v) ethanol. GAF extraction and concentration was performed as described in the legend to Table 3, except that the dry extract was redissolved in a volume of 0.05 M KH₂PO₄ buffer (pH 6.5, KOH) equal to one twentieth of the original culture filtrate volume. FIG. 3B—As a control, Arabidopsis seeds were sown onto 2% agar containing 10% (by volume) of the Pseudomonas Minimal Salts Medium (see Example 1 for formulary). Seeds of Poa annua sown onto agar containing the GAF concentrate served as a positive control.

FIG. 4 is a series of digital images showing the time course of GAF effects on Poa annua seed germination. Seeds of P. annua were allowed to imbibe water for 48 hours, then were exposed to WH6 filtrate for 1, 4, 8, 12, 20, or 24 hours. Each seed was then thoroughly rinsed with water to remove residual treatment and placed in a growth chamber under conditions favorable to seed germination (20° C., eight hour photoperiod, 50 μM/m²/s). These digital images demonstrate the effects of treatment after exposure to WH6 filtrate for the indicated time. Bar=1 mm.

FIG. 5 is a series of digital images showing the effects of GAF extracts derived from Pseudomonas fluorescens WH6 on the germination of the seeds of Poa annua in soil systems. Woodburn sandy loam soil was passed through a screen (0.3 cm) and dried overnight at 37° C. prior to use. FIGS. 5A and 5B illustrate the GAF response of Poa annua seed treated on Woodburn sandy-loam soil saturated with a 5× GAF concentrate prepared as described in the legend to Table 5. The digital images were made seven days after treatment. Bar=1 mm. FIG. 5A shows GAF-treated seeds. FIG. 5B shows untreated control seeds. FIGS. 5C and 5D illustrate the GAF response of Poa annua seed allowed to imbibe water for 5 days on flats of moist Woodburn sandy-loam soil and then sprayed with a 4× GAF concentrate, prepared as described in the legend to Table 9, except that the dried 90% ethanol extract was reconstituted in distilled water containing 0.1% (v/v) Tween-20. The spray treatment was repeated after two days. Controls were sprayed with water containing 0.1% (v/v) Tween-20 using the same time intervals. After spraying the flats were maintained at 15° C. with 50 μmol/m²/s light and misted with water as needed. FIG. 5C shows the GAF-treated flats seven days after the first spray treatment FIG. 5D shows the control flats seven days after the first spray treatment FIGS. 5E and 5F show close-up views of the same flats.

FIG. 6 is a pair of digital images showing the effects of GAF extracts derived from Pseudomonas fluorescens WH6 on the germination of the seeds Aegilops cylindrica in soil systems. These images illustrate the GAF response of Aegilops cylindrica seeds treated (as described in the legend to Table 6) on Woodburn sandy-loam soil saturated with a 5× GAF concentrate. The images were made seven days after treatment. Bar=1 mL FIG. 6A shows GAF-treated seeds. FIG. 6B shows untreated control seeds.

FIG. 7 is a graph showing the result of gel filtration chromatography of WH6 GAF activity on a Sephadex G-10 column A sample (19 ml) of Pseudomonas fluorescens WH6 culture filtrate was taken to dryness in vacuo at 45° C., and the dry solids were extracted three times with 90% (v/v) ethanol (6.3 ml for five minutes per each extraction). The combined ethanol extracts were dried in vacuo at (45° C.) and redissolved in 3.8 ml of distilled water. A 3.0 ml aliquot of this solution was loaded on a Sephadex G-10 column (1.5×41 cm, 30 grams of Sephadex G-10, ca. 68 ml bed volume) that had been packed and equilibrated in distilled water. The G-10 column was eluted with distilled water, and fractions (3.0 ml) were collected. The pH of each fraction was adjusted to 6.0 by the addition of 0.3 ml of 0.5 M KH₂PO₄ buffer (ph 6.0, KOH), and samples of each fraction were tested for GAF activity in the Standard GAF Bioassay System (see Example 1) using seeds of annual bluegrass (Poa annua). Following chromatography of the GAF sample, the column void volume was determined by chromatographing a sample of Blue Dextran on the column.

FIG. 8 is a graph showing the results of a test of the retention of WH6 GAF activity by an anion-exchange column (QAE-Sephadex). A sample (18 ml) of Pseudomonas fluorescens WH6 culture filtrate was taken to dryness in vacuo at 45° C., and the dry solids were extracted three times with 90% (v/v) ethanol (6 ml for five minutes per each extraction). The combined ethanol extracts were dried in vacuo (45° C.) and redissolved in 12 ml of 0.025 M Tris-HCl buffer (pH.9, HCl). A 1 ml aliquot of this solution was taken for a bioassay sample, and the remaining 11 ml sample was loaded on a QAE-Sephadex column (1.5×7.5 cm, 2 grams of QAE-Sephadex, ca. 13.3 ml bed volume) equilibrated with the same buffer. (The QAE-Sephadex column had been packed and washed overnight with the same buffer containing 0.5 M KCl, then washed with buffer without KCl, prior to loading the sample). After the sample was loaded, the column was washed with 72 ml of the equilibration buffer, followed by 72 ml of 0.05 M KH₂PO₄ (pH 5.6, KOH). The column eluate was collected in 12 ml fractions. The pH of each fraction was adjusted to ca. 6.3, and samples of each fraction were tested for GAP activity in the Standard GAF Bioassay System (see Example 1) using seeds of annual bluegrass (Poa annua).

FIG. 9 is a graph showing the results of a test of the retention of WH6 GAF activity by a cation-exchange column (SP-Sephadex). A sample (18 ml) of Pseudomonas fluorescens WH6 culture filtrate was taken to dryness in vacuo at 45° C., and the dry solids were extracted three times with 90% (v/v) ethanol (6 ml for five minutes per each extraction). The combined ethanol extracts were dried in vacuo (45° C.) and redissolved in 12 ml of 0.025 M KH₂PO₄ buffer (pH 3.8 KOH). A 1 ml aliquot of this solution was taken for a bioassay sample, and the remaining 11 ml sample was loaded on a SP-Sephadex column (1.5×8.4 cm, 2 grams of QAE-Sephadex, ca. 14.8 ml bed volume) equilibrated with the same buffer. (The SP-Sephadex column had been packed and washed overnight with the same buffer prior to loading the sample.) After the sample was loaded, the column was washed with 78 ml of the equilibration buffer, followed by 65 ml of 0.05 M KH₂PO₄ (pH 8.2, KOH). The column eluate was collected in 13 ml fractions. The pH of each fraction was adjusted to ca. 6.1, and samples of each fraction were tested for GAF activity in the Standard GAF Bioassay System (see Example 1) using seeds of annual bluegrass (Poa annua).

FIG. 10 is a set of digital images showing the results of thin-layer chromatography (TLC) of 90% (v/v) ethanol extracts of WH6 culture filtrates. FIG. 10A is a digital image of a TLC plate viewed under ultraviolet light Chromatographic samples were prepared from Pseudomonas fluorescens WH6 culture filtrates as described in Example 1. The samples were chromatographed on Avicel Microcrystalline Cellulose TLC plates and analyzed as described under the same reference. FIG. 10B is a schematic showing the distribution of GAF activity on the TLC plate shown in FIG. 10A. Cellulose was recovered from the indicated chromatographic zones and analyzed for GAF activity as described in Methods. The zones of GAF activity are indicated with Germination Scores as defined in Table 5. FIG. 10C is a digital image showing ninhydrin staining of a duplicate TLC plate. The TLC plate was developed and sprayed with ninhydrin as described in Example 1.

FIG. 11 is a set of digital images showing a comparison of the ninhydrin staining of thin-layer chromatograms of 90% ethanol extracts of culture filtrates prepared from wild type WH6, from the gaf1 mutant of WH6, and from the gaf1 mutant after restoration of GAF activity by complementation with the wild type GAF1 gene. FIG. 11A is a digital image of the ninhydrin staining of a TLC chromatogram of an extract of a culture filtrate prepared from wild type WH6 culture filtrate. FIG. 11B is a digital image of the ninhydrin staining of a TLC chromatogram of an extract of a culture filtrate prepared from the gaf1 mutant FIG. 11C is a digital image of the ninhydrin staining of a TLC chromatogram of an extract of a culture filtrate prepared from the gaf1 mutant following complementation with wild type GAF1 gene. Chromatographic samples (90% ethanol extracts of culture filtrates derived from wild type Pseudomonas fluorescens WH6, from the gaf1 mutant of WH6, and from the gaf1 mutant following complementation with the wild type GAF1 gene) were prepared as described in Example 1. The samples were chromatographed on Avicel Microcrystalline Cellulose TLC plates and analyzed as described under the same reference. Arrows identify the ninhydrin positive zone present in culture filtrates prepared from WH6 and the gaf1 mutant following complementation with the wild type GAF1 gene but absent in the gap mutant. This zone corresponds to the position of GAF activity in chromatograms tested in the Standard GAF Bioassay System.

FIG. 12 is a set of digital images showing a comparison of the ninhydrin staining of thin-layer chromatograms of 90% ethanol extracts of culture filtrates prepared from wild type WH6, from the gaf2 mutant of WH6, and from the gaf2 mutant after restoration of GAF activity by complementation with the wild type GAF2 gene. FIG. 12A is a digital image of the ninhydrin staining of a LC chromatogram of an extract of a culture filtrate prepared from wild type WH6 culture filtrate. FIG. 12B is a digital image of the ninhydrin staining of a TLC chromatogram of an extract of a culture filtrate prepared from the gaf2 mutant FIG. 12C is a digital image of the ninhydrin staining of a TLC chromatogram of an extracts of a culture filtrate prepared from the gaf2 mutant following complementation with wild type GAF2 gene. Chromatographic samples (90% ethanol extracts of culture filtrates derived from wild type Pseudomonas fluorescens WH6, from the gaf2 mutant of WH6, and from the gaf2 mutant following complementation with the wild type GAF2 gene, were prepared as described in Example 1. The samples were chromatographed on Avicel Microcrystalline Cellulose TLC plates, and the developed plates were analyzed as described under the same reference. Arrow identifies the ninhydrin positive zone present in the extract of culture filtrates from WH6 and the gaf2 mutant following complementation with the wild type GAF2 gene, but absent in the gaf2 mutant. The zone corresponds to the position of GAF activity in chromatograms tested in the Standard GAF Bioassay System.

FIG. 13 is a set of digital images showing a comparison of the ninhydrin staining of thin-layer chromatograms of 90% ethanol extracts of culture filtrates prepared from wild type WH6, from the gaf3 mutant of WH6, and from the gaf3 mutant after restoration of GAF activity by complementation with the wild type GAF3 gene. FIG. 13A is a digital image of the ninhydrin staining of a TLC chromatogram of an extract of a culture filtrate prepared from wild type WH6 culture filtrate. FIG. 13B is a digital image of the ninhydrin staining of a TLC chromatogram of an extract of a culture filtrate prepared from the gaf3 mutant FIG. 13C is a digital image of the ninhydrin staining of a TLC chromatogram of an extract of a culture filtrate prepared from the gaf3 mutant following complementation with wild type GAF3 gene. Chromatographic samples (from culture filtrates derived from wild type Pseudomonas fluorescens WH6, from the gaf3 mutant of WH6, and from the gaf3 mutant following complementation with the wild type GAF3 gene) were prepared as described in Example 1. The samples were chromatographed on Avicel Microcrystalline Cellulose TLC plates, and the developed plates were analyzed as described under the same reference. Arrows identify the ninhydrin positive zone present in culture filtrates from WH6 and the gaf3 mutant following complementation with wild type GAF3 gene but absent in the gaf3 mutant. This zone corresponds to the position of GAF activity in chromatograms tested in the Standard GAF Bioassay System.

### DETAILED DESCRIPTION

### I. Abbreviations

ExPASy: Expert Protein Analysis System

FAME: Fatty Acid Methyl Ester Analysis

FPM: Fluorescent Pseudomonas Medium

GAF: Germination-Arrest Factor

h: hour

OD: Optical Density

ORF: Open Reading Frame

mL: milliliter

μM: micromolar

mm: millimeter

mm: millimolar

m²: square meters

PCR: Polymerase Chain Reaction

PMS: Pseudomonas Minimal Salt Medium

QSAR: Quantitative Structure Activity Relationships

s: second

SCFU: Single Colony Forming Units

SIB: Swiss Institute of Bioinformatics

TLC: thin-layer chromatography

UV: Ultraviolet

v/v: volume-to-volume

w/v: wieght-to-volume

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided.

Adjuvant: A chemical added to an herbicide formulation or tank mix to improve mixing and application or enhance performance. Most herbicide formulations contain at least a small percentage of adjuvants. Wetting agents and spreaders are the adjuvants most frequently added. Common adjuvants include, but are not limited to, wetting agents, such as anionic, cationic, nonionic, and amphoteric surfactants, stabilizers, preservatives, antioxidants, extenders, solvents, emulsifiers, invert emulsifiers, spreaders, stickers, penetrants, foaming agents, anti-foaming agents, thickeners, safeners, compatibility agents, crop oil concentrates, viscosity regulators, binders, tackers, drift control agents, or other chemical agents, such as fertilizers, antibiotics, fungicides, nematicides, or pesticides.

Annual Bluegrass (Poa annua): A low-growing, cool season grass that dies early in the summer when the top soil dries out Annual bluegrass can be distinguished easily from other grasses by its typical leaf tip, which is shaped like the bow of a boat. In addition, the leaf blade is often crinkled at midsection. The mature plant grows as dense, low-spreading tufts, 8 to 30 cm tall, and often roots at the lower nodes.

Annual bluegrass begins to emerge in late summer and early fall when night temperatures are in the 60's and moisture is abundant. Annual bluegrass seeds continue to germinate through the fall, winter and spring, a characteristic that makes chemical control more difficult Germination and growth of annual bluegrass are favored by moist soil conditions and cool temperatures. Thus, it has a strong competitive advantage over warm season grasses from fall through spring. On closely mowed and irrigated turf, annual bluegrass will dominate a stand of bermuda grass by late spring if herbicides are not used. Populations of annual bluegrass are greatly reduced by taller mowing heights and limited use of water.

Annual bluegrass initiates seedheads in late fall and winter, but seedhead development is greatest in the spring and early summer. After seedhead appearance, the turf develops a yellowish-white color and an uneven appearance.

Annual bluegrass is best controlled with pre- and post-emergence herbicides in warm season turf grasses. Preemerge products generally are applied prior to the emergence of annual bluegrass in the fall. The date of emergence varies between locations and years, but generally begins when night temperatures are in the 60's and daytime temperatures are below 85° F. Where winter grasses are to be overseeded on bermuda grass turf, applications of preemergence products generally are made 60 to 90 days before seeding. Annual bluegrass also can be controlled using the compounds and methods described herein.

Antibiotic: A substance, for example penicillin or streptomycin, often produced by or derived from certain fungi, bacteria, and other organisms, that can destroy or inhibit the growth of other microorganisms.

Anti-foaming agent: An adjuvant that reduces foaming of spray mixtures that require vigorous agitation.

Antioxidant: An adjuvant, such as vitamin E, vitamin C, or beta carotene that protects a compound, for example an herbicide, from the damaging effects of oxidation.

Antisense, Sense, and Antigene: Double-stranded DNA (dsDNA) has two strands, a 5′->3′ strand, referred to as the plus strand, and a 3′->5′ strand (the reverse compliment), referred to as the minus strand. Because RNA polymerase adds nucleic acids in a 5′ a 3′ direction, the minus strand of the DNA serves as the template for the RNA during transcription. Thus, the RNA formed will have a sequence complementary to the minus strand and identical to the plus strand (except that U is substituted for T).

Antisense molecules are molecules that are specifically hybridizable or specifically complementary to either RNA or plus strand DNA. Sense molecules are molecules that are specifically hybridizable or specifically complementary to the minus strand of DNA. Antigene molecules are either antisense or sense molecules complimentary to a dsDNA target. In one embodiment, an antisense molecule specifically hybridizes to a target mRNA and inhibits transcription of the target mRNA.

Bioherbicide: A preparation of living inoculum of a plant pathogen or other microorganism, or a compound produced by such a pathogen or other microorganism, that is formulated and applied in a manner analogous to that of a chemical herbicide in an effort to suppress the growth of weed species. The use of bioherbicides is based on the fundamental epidemiological principles of plant pathology.

Pathogen factors such as low inoculum levels, weakly virulent pathogens, and poor spore dispersal mechanisms; environmental factors such as unfavorable moisture and/or temperature conditions; and plant factors such as low susceptibility of the host, and widely dispersed host populations often limit disease. The bioherbicide approach is an attempt to bypass many of these restraints on disease development by periodically dispersing an abundant supply of virulent inoculum, or a compound produced by a plant pathogen or other microorganism, uniformly onto a susceptible weed population. Generally, the application is timed to take advantage of favorable environmental conditions and/or the most susceptible stage of plant growth. Similarly, the bioherbicide generally is formulated to avoid unfavorable environmental conditions and to facilitate application.

Buffer: An ionic compound that resists changes in its pH.

cDNA (complementary DNA): A piece of DNA lacking internal, non-coding segments (introns) and transcriptional regulatory sequences. cDNA can also contain untranslated regions (UTRs) that are responsible for translational control in the corresponding RNA molecule. cDNA is synthesized in the laboratory by reverse transcription from messenger RNA extracted from cells.

Compatibility Agent: An adjuvant that aids in combining herbicides effectively.

Crop Oil Concentrate: A phytobland petroleum or vegetable oil that increases absorption of an herbicide through the cuticle of leaves.

Dicot: Any flowering plant with two embryonic seed leaves (cotyledons). Cotyledons usually appear at germination.

DNA (deoxyribonucleic acid): A long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. Thus, for instance, it is appropriate to generate probes or primers from the reverse complement sequence of the disclosed nucleic acid molecules.

Downy Brome (Bromus tectorum): An erect winter- or spring-annual grass. The seedlings are bright green with conspicuously hairy leaves, hence the common name, downy brome. At maturity, the foliage and seedheads often become purplish before drying completely and becoming brown or tan. The species grows quickly in the spring and often matures and sets seeds before most other species. It typically grows 50-60 cm (20-24 inches) tall, with a finely divided, fibrous root system that may reach a depth of about 30 cm (12 inches). When environmental conditions are poor and/or when grazing animals crop the plants, downy brome plants that reach heights of just 5-10 cm (2-4 in) can still flower and produce viable seed. The stems are erect, slender, and glabrous or may be slightly soft and hairy. The nodding, open panicles with moderately awned spikelets are very distinctive. The spikelets readily penetrate fur, socks and pants and its seeds may thus be widely dispersed by people and animals.

Drift Control Agent: An adjuvant used to reduce the fine particles in the spray pattern that are responsible for herbicide drift and crop injury.

Dry flowable: A granule formulation much like a wettable powder, except that the active ingredient is formulated on a large particle (granule) instead of onto a ground powder. This type of formulation offers essentially the same advantages and disadvantages as wettable powder formulations. However, these formulations generally are more easily mixed and measured than wettable powders. Because they create less dust when handling, they cause less inhalation hazard to the applicator during pouring and mixing.

Emulsifiable concentrate: A liquid herbicide formulation that contains the active ingredient, one or more solvents, and an emulsifier that allows mixing with water. Formulations of this type are highly concentrated, relatively inexpensive per pound of active ingredient, and easy to handle, transport, and store. In addition, they require little agitation (will not settle out or separate) and are not abrasive to machinery or spraying equipment.

Emulsifier: A substance that promotes the suspension of one liquid in another. Emulsifiers are often used to disperse petroleum-based herbicides in water.

Encode: A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

Extender: An adjuvant added to an herbicide to modify, dilute, or adulterate it.

Foaming agent: An adjuvant used to reduce foaming in a spray system so that pumps and nozzles can operate properly.

Functional fragments and variants of a polypeptide: Included are those fragments and variants that maintain one or more functions of the parent polypeptide. It is recognized that the gene or cDNA encoding a polypeptide can be considerably mutated without materially altering one or more the polypeptide's functions. First, the genetic code is well-known to be degenerate, and thus different codons encode the same amino acids. Second, even where an amino acid substitution is introduced, the mutation can be conservative and have no material impact on the essential functions of a protein. See Stryer, Biochemistry 3rd Ed., (c) 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al., 1997). Other modifications that can be made without materially impairing one or more functions of a polypeptide include, for example, in vivo or in vitro chemical and biochemical modifications or the incorporation of unusual amino acids. Such modifications include, for example, acetylation, carboxylation, phosphorylation, glycosylation, ubiquination, labeling, e.g., with radionucleides, and various enzymatic modifications, as will be readily appreciated by those well skilled in the art. A variety of methods for labeling polypeptides and labels useful for such purposes are well known in the art, and include radioactive isotopes such as ³²P, ligands which bind to or are bound by labeled specific binding partners (e.g., antibodies), fluorophores, chemiluminescent agents, enzymes, and antiligands. Functional fragments and variants can be of varying length. For example, some fragments have at least 10, 25, 50, 75, 100, 200, or even more amino acid residues.

Fungicide: A chemical substance that destroys or inhibits the growth of fungi.

Germination-Arrest Factor (GAF): A factor that inhibits or arrests seed germination. In some embodiments of the disclosure, GAF is a highly specific, effective, naturally occurring bioherbicide that is an alternative to chemical herbicides (e.g., Diuron) for the control of grassy weeds. GAF was identified as the product of particular isolates of certain rhizobacteria (Pseudomonas species; see Example 2) obtained from Willamette Valley soils in Oregon. GAF irreversibly arrests the germination of the seeds of a number of grassy weeds and some cultivated grasses, for example, but not limited to, annual bluegrass, downy brome, jointed goatgrass, roughstalk bluegrass, rattail fescue, and cultivated perennial ryegrass, and tall fescue, in addition to both domestic and wild oats. GAF does not arrest the germination of seeds of wheat, corn, barley, or the dicot species tested to date.

GAF has a unique mode of action that distinguishes it from less selective inhibitors of seed germination. Sensitive seeds initiate germination in the presence of GAF but seedling development is irreversibly arrested immediately following emergence of the coleorhiza and plumule. Thus, sensitivity to GAF is both species-specific and developmentally specific. Established seedlings appear to have little if any sensitivity to GAF, offering unique opportunities for weed management in grass cropping systems. Furthermore, culture filtrates containing GAF can be diluted, for example, filtrates can be diluted two- or three-fold, or even as much as ten-fold, or even more with little or no loss of activity. Thus, GAF is active at very low absolute concentrations.

An “effective amount” of GAF refers to an amount that has an adverse biological effect on at least some of the seeds exposed to the GAF. For example, the effective amount of GAF may be an amount sufficient to arrest germination of at least some weed seeds in a seed population. In specific embodiments of the disclosure, an effective amount of a GAF arrests germination in at least 10% of the seeds treated. In particular embodiments of the disclosure, an effective amount of GAF arrests germination in at least 20%, or even 50%, of a seed population. In more particular embodiments of the disclosure, an effective amount of a GAF arrests germination in over 90%, or nearly 100% of a seed population. Specific examples of effective amounts of GAF are provided in the Examples below.

Germination-Arrest Factor Proteins or Peptides: GAF proteins are defined herein as polypeptides that either function directly to inhibit or arrest germination of grassy weeds, or are required for the synthesis or secretion of a Germination Arrest Factor. GAF peptides are defined as functional fragments or variants of GAF proteins that are capable of either functioning directly to inhibit or arrest germination of grassy weeds, or are required for the synthesis or secretion of a Germination Arrest Factor.

Grassy Weed: Any of various undesirable plants having slender leaves characteristic of the grass family, for example, but not limited to crabgrass, goosegrass, dallisgrass, bahiagrass, annual bluegrass, downy brome, jointed goatgrass, roughstalk bluegrass, rattail fescue, perennial ryegrass, and tall fescue. Grassy weeds are troublesome in lawns, sports fields, playgrounds, golf courses and other turf grass areas because they are unsightly and lead to poor playing conditions. In addition, grassy weeds greatly increase mowing costs and present a safety hazard on sports fields and playgrounds.

Granule: An herbicide formulation that is used for soil-applied herbicides. In a granule formulation, the active ingredient is formulated onto large particles (granules). The primary advantages of this type of formulation are that the formulation is ready to use with simple application equipment (seeders or spreaders), and the drift potential is low because the particles are large and settle quickly. The disadvantages of this formulation are that it does not adhere to foliage (not intended for foliar applications), and may require mixing into the soil in order to achieve adequate herbicidal activity.

Herbicide: A compound or composition that produces an adverse effect on a plant, including (but not limited to) physiological damage to the plant; inhibition or modulation of plant growth; inhibition or modulation of plant reproduction; or death of the plant Exemplary examples of herbicides include, but are not limited to, photosystem II inhibitors, protox inhibitors and superoxide generators, glufosinate, cell growth disruptors, cell growth inhibitors, lipid biosynthesis inhibitors, growth regulator herbicides, pigment inhibitors, inhibitors of amino acid biosynthesis, and inhibitors of cell wall biosynthesis.

To be effective, herbicides generally must 1) adequately contact plants; 2) be absorbed by plants; 3) move within the plants to the site of action, without being deactivated; and 4) reach toxic levels at the site of action. The application method used, whether preplant incorporated, preemergence, or postemergence, determines whether the herbicide will contact germinating seedlings, roots, shoots, or leaves of plants.

The term “mode of action” refers to the sequence of events from absorption into plants to plant death. The mode of action of the herbicide influences how the herbicide is applied. For example, contact herbicides that disrupt cell membranes, such as acifluorfen (Blazer) or paraquat (Gramoxone Extra), need to be applied postemergence to leaf tissue in order to be effective. Seedling growth inhibitors, such as trifluralin (Treflan) and alachlor (Lasso), need to be applied to the soil to effectively control newly germinated seedlings.

Because the seeds of many weed species are quite small and germinate within 0.5 to 1.0 inch of the soil surface, soil-applied herbicides generally are positioned in the top 1 to 2 inches of soil. Herbicide positioning can be accomplished by mechanical incorporation or by rainfall. Once an herbicide comes into contact with a plant, absorption through the roots or shoots occurs. An herbicide that is absorbed through the roots generally will be taken up as long as the herbicide-treated soil remains in contact with the absorbing region near the root tips. As the roots grow to greater soil depths, herbicide uptake generally declines. Therefore, weeds not killed before the root tips grow out of the herbicide-treated soil likely will survive.

Many soil-applied herbicides are absorbed through plant shoots while they are still underground and may kill or injure the shoots before they emerge from the soil. Volatile herbicides such as the thiocarbamates (e.g., EPTC [Eradicane]) and the dinitroanilines (e.g., trifluralin [Treflan]) can penetrate the shoot as gases. Less volatile herbicides such as the acetanilides (e.g., alachlor [Lasso]) are absorbed into the shoot as liquids. Physical and environmental factors that promote rapid crop emergence reduce the length of time that a plant is in contact with a soil-applied herbicide and, therefore, reduce the possibility of crop injury.

Herbicides differ in their ability to translocate within a plant. The soil-applied dinitroaniline herbicides (e.g., trifluralin [Treflan]) are not mobile within the plant Therefore, their injury symptoms are confined to the site of uptake. Other herbicides are mobile within the plant. For example, soil-applied atrazine is absorbed by plant roots and moves upward within the xylem of the plant to be concentrated in the leaves. In general injury symptoms will be most prominent at the site where the mobile herbicides concentrate.

Effective postemergence herbicide application is dependent upon adequate contact with above-ground plant shoots and leaves. Therefore, spray pressure, nozzle type, and volume generally are adjusted for adequate plant coverage.

For postemergence herbicides, the chemical and physical relationships between the leaf surface and the herbicide often determine the rate and amount of uptake. Factors such as plant size and age, water stress, air temperature, relative humidity, and herbicide additives can influence the rate and amount of herbicide uptake. Additives such as crop oil concentrates, surfactants, or liquid fertilizer solutions (e.g., UAN) can increase herbicide uptake by a plant. Application of herbicides under hot and dry conditions or application to older and larger weeds or weeds under water stress can decrease the amount of herbicide uptake. Differences in the rate and amount of herbicide uptake influence the potential for crop injury and weed control and often explain the year to year variation in the effectiveness of the herbicide. In addition, the faster an herbicide is absorbed by a plant, the less likely it will be that rain will wash the herbicide off the plants.

Like soil-applied herbicides, postemergence herbicides differ in their ability to move within a plant For adequate weed control nonmobile postemergence herbicides generally must thoroughly cover the plant. Nonmobile herbicides are often called contact herbicides, and include the bipyridylium, diphenylether, benzothiadiazole, and nitrile families. Other herbicides are mobile within the plant and can move from the site of application to their site of herbicidal activity. For example, growth regulator herbicides such as 2,4-D and dicamba (Banvel) move both upward and downward within a plant's phloem to the growing points of the shoots and roots. In general, injury symptoms will be most prominent at the sites at which the mobile herbicides concentrate.

Plants that can rapidly degrade or deactivate an herbicide can escape that herbicide's toxic effects. For example, corn is tolerant to the triazine herbicides because it quickly deactivates these herbicides by binding them to naturally occurring plant chemicals. Soybean tolerance to metribuzin (Sencor, Lexone) is at least partially due to the deactivation of the herbicide by conjugating to plant sugar molecules.

Furthermore, a crop may be injured by an herbicide to which it is normally tolerant. This often occurs because environmental stresses such as hot or cold temperatures, high relative humidity, or hail decrease a plant's natural ability to reduce herbicide uptake or deactivate an herbicide. Postemergence cyanazine (Bladex) injury to corn under cold and wet weather conditions is an example of environmentally induced herbicide injury. An excessive application of herbicide, due to misapplication, can also injure a tolerant crop by overwhelming the crop's herbicide degradation and deactivation systems.

A number of weed species that were once susceptible and easily managed by certain herbicides have now developed resistance to these agents. These weeds are no longer controlled by applications of previously effective herbicides. To date, at least 53 species of weeds are resistant to at least five different herbicide families.

Invert emulsifier: An adjuvant that allows water-based herbicides to mix with a petroleum carrier.

Isolated: An isolated biological component (such as a nucleic acid molecule or protein) is one that has been substantially separated or purified away from other biological components in the cell of the organism in which the component naturally occurs, i.e., other chromosomal and extra-chromosomal DNA and RNA, proteins and organelles. Nucleic acids and proteins that have been isolated include nucleic acids and proteins purified by standard purification methods. The term also embraces nucleic acids and proteins prepared by recombinant expression in a host cell as well as chemically synthesized nucleic acids.

Jointed Goatgrass (Aegilops cylindrica): A native of southern Europe and western Asia, jointed goatgrass is so closely related to wheat that both species can interbreed. It is difficult to distinguish jointed goatgrass from wheat until spikes appear. Jointed goatgrass spreads exclusively by seed, and grows best in cultivated fields, but can also invade grasslands.

Jointed goatgrass is a winter annual, but about five percent of a population may be spring annuals. Leaves are grasslike, up to a half-inch wide, and have evenly spaced fine hairs along the leaf edges and down the sheath opening. The ligule is short and membranous; auricles are short and hairy. Stems can grow up to four feet tall and are tipped with slender, cylindrical spikes that appear to be a series of joints stacked on top of each other. Reddish to straw-colored spikes emerge in May to June, and uppermost joints are tipped by straight awns. Up to three seeds are enclosed in each joint.

Jointed goatgrass is found in all major United States winter wheat production regions, from Texas to South Dakota and eastern Montana, and in portions of the Northwest and Utah. No herbicides are available that can selectively control jointed goatgrass in winter wheat, however spring tillage and general grass killers provide good control.

Liquid flowable: An herbicide formulation made up of finely ground active ingredient suspended in a liquid. Flowables generally are mixed with water for application, are easily handled and applied, and seldom clog nozzles. Some of their disadvantages are that they may leave a visible residue on plant and soil surfaces, and typically require constant and thorough agitation to remain in suspension.

Nematicide: A substance or preparation used to kill nematodes.

Ninhydrin: A chemical compound with the molecular formula C₉H₆O₄, ninhydrin is also known as ninhydrin monohydrate, 1,2,3-triketohydrindene monohydrate, 1,2,3-indantrione monohydrate, 2,2-dihydroxy-1,3-indandione, 1H-indene-1,2,3-trione monohydrate. Ninhydrin produces a purple reaction product in the presence of primary amines. A ninhydrin-positive or ninhydrin-reactive agent is one that produces such a reaction product, and the presence of such a reaction product indicates that the agent includes at least one primary amine. Thus, a ninhydrin-reactive agent is one that includes at least one primary amine, for example a peptide or protein, or an another agent that includes at least one primary amine, such as an enzymatically synthesized or modified agent

Nucleic Acid: A deoxyribonucleotide or ribonucleotide polymer in either single or double stranded form, and unless otherwise limited, encompasses known analogues of natural nucleotides that hybridize to nucleic acids in a manner similar to naturally occurring nucleotides.

Nucleotide: This term includes, but is not limited to, a monomer that includes a base linked to a sugar, such as a pyrimidine, purine or synthetic analogs thereof, or a base linked to an amino acid, as in a peptide nucleic acid. A nucleotide is one monomer in a polynucleotide. A nucleotide sequence refers to the sequence of bases in a polynucleotide.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid molecules.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15, or 20 bases.

Open reading frame: A series of nucleotide triplets (codons) coding for amino acids without any internal termination codons. These sequences are usually translatable into a peptide.

Pellet: An herbicide formulation used for soil-applied herbicides. In a pellet formulation, the active ingredient is formulated onto large particles (pellets). The primary advantages of this type of formulation are that the formulation is ready to use with simple application equipment (seeders or spreaders), and the drift potential is low because the particles are large and settle quickly. The disadvantages of this formulation are that it does not adhere to foliage (not intended for foliar applications), and may require mixing into the soil in order to achieve adequate herbicidal activity.

Penetrant: An adjuvant that allows an herbicide to get through the outer surface to the inside of a treated area.

Perennial Ryegrass (Lolium perenne): A perennial grass that grows to 8-90 centimeters tall, and has loose to densely tufted, short-lived, glabrous leaves. Perennial ryegrass can be decumbent or (rarely) prostrate, sometimes rooting at lowest nodes, usually with two to four nodes below spike. Basal leaf-sheaths are green, reddish, purplish, or straw-colored, and leaf-blades are folded in young shoots. Mature blades are acute, attenuate, or somewhat rounded at apices, usually less than 14 centimeters long, and one to six millimeters broad. Auricles typically are present, but may be absent, and are minute, up to three millimeters long; bearing 5-37 spikelets.

Pesticide: A chemical used to kill pests, for example insects.

Polypeptide: A polymer in which the monomers are amino acid residues which are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

The term polypeptide fragment refers to a portion of a polypeptide that exhibits at least one useful epitope. The phrase functional fragment of a polypeptide refers to all fragments of a polypeptide that retain an activity, or a measurable portion of an activity, of the polypeptide from which the fragment is derived. Fragments, for example, can vary in size from a polypeptide fragment as small as an epitope capable of binding an antibody molecule to a large polypeptide capable of participating in the characteristic induction or programming of phenotypic changes within a cell.

The term substantially purified polypeptide as used herein refers to a polypeptide that is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Preservative: An adjuvant that inhibits degradation of an herbicide.

Probes and primers: Nucleic acid probes and primers can be readily prepared based on the nucleic acid molecules provided in this disclosure. A probe comprises an isolated nucleic acid attached to a detectable label or reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, e.g., in Sambrook et al. (In Molecular Cloning: A Laboratory Manual, CSHL, New York, 1989) and Ausubel et al. (In Current Protocols in Molecular Biology, Greene Publ. Assoc. and Wiley-Intersciences, 1992).

Primers are short nucleic acid molecules, preferably DNA oligonucleotides 10 nucleotides or more in length. More preferably, longer DNA oligonucleotides can be about 15, 17, 20, or 23 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al (In Molecular Cloning: A Laboratory Manual, CSHL, New York, 1989), Ausubel et al (In Current Protocols in Molecular Biology, Greene Publ. Assoc. and Wiley-Intersciences, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular probe or primer increases with its length. Thus, in order to obtain greater specificity, probes and primers can be selected that comprise at least 17, 20, 23, 25, 30, 35, 40, 45, 50 or more consecutive nucleotides of GAF nucleotide sequences.

Protein: A biological molecule expressed by a gene and comprised of amino acids.

Purified: The term purified does not require absolute purity; rather, it is intended as a relative term. Thus, for example, a purified protein preparation is one in which the protein referred to is more pure than the protein in its natural environment within a cell.

Rattail Fescue (Vulpia myuros): Rattail fescue is an annual grassy weed. It grows to be up to about two feet tall. Rattail fescue exhibits narrow leaf blades, which are folded and hairless. Panicles are slender and up to eight inches in length, and awns are about 5/16 to ⅜ inch long.

Roughstalk Bluegrass (Poa trivialis): A perennial bluegrass that grows from stolons that may reach about one to three feet in height. These plants go dormant throughout the summer and carry out their life cycle during the winter months.

Roughstalk bluegrass stems are covered with many small hairs, with brown to purple bands surrounding the nodes. Leaves are folded in the bud and have the boat-shaped tip typical of most bluegrass species. Leaf blades are about two to seven inches long, and about two to five millimeters wide. They are covered with many small hairs, and have a relatively large (about four to six millimeters) membranous ligule. The seedhead is a panicle very similar to other bluegrass turf species.

Roughstalk bluegrass produces fibrous roots with a stoloniferous system that contributes significantly to the spread of this weed. As an agronomic weed, roughstalk bluegrass is relatively easy to distinguish from other grasses. The distinctive boat-shaped leaf tip, seedhead, growing season, and presence of stolons are all characteristics that aid in identification.

Safener: An adjuvant that reduces the toxicity of an herbicide formulation to the herbicide handler or to the crop.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of a GAF protein, and the corresponding cDNA sequence, will possess a relatively high degree of sequence identity when aligned using standard methods. This homology will be more significant when the orthologous proteins or cDNAs are derived from species that are more closely related, compared to species more distantly related.

Typically, orthologs are at least 50% identical at the nucleotide level and at least 50% identical at the amino acid level when comparing GAF genes and proteins to orthologous GAF genes and proteins.

Methods of alignment of sequences for comparison are well known in the art. Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene, 73: 237-244, 1988; Higgins & Sharp CABIOS 5:151-153, 1989; Corpet et al. Nuc. Acids Res. 16, 10881-10890, 1988; Huang et al. Computer Appls. in the Biosciences 8, 155-165, 1992; and Pearson et al. Meth. Mol. Bio. 24, 307-331, 1994. Altschul et al. (J. Mol. Biol. 215:403-410, 1990) present a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI website, together with a description of how to determine sequence identity using this program.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence remains hybridized to a perfectly matched probe or complementary strand. Conditions for nucleic acid hybridization and calculation of stringencies can be found in Sambrook et al. (1989) Molecular Cloning: A Laboratory Manual, CSHL, New York and Tijssen (1993) Laboratory Techniques in Biochemistry and Molecular Biology-Hybridization with Nucleic Acid Probes Part I, Chapter 2, Elsevier, New York.

Nucleic acid sequences that do not show a high degree of identity can nevertheless encode similar amino acid sequences, due to the degeneracy of the genetic code. It is understood that changes in nucleic acid sequence can be made using this degeneracy to produce multiple nucleic acid molecules that all encode substantially the same protein.

Soluble powder: An herbicide formulation that, when mixed with water, dissolves readily and forms a true solution. Soluble powder formulations are dry and include the active ingredient and additives. When thoroughly mixed, no further agitation is necessary to keep the active ingredient dissolved in solution.

Solution: A liquid formulation that includes an active ingredient and an additive. Solution formulations are designed for those active ingredients that dissolve readily in water. Generally, when herbicides formulated as solutions are mixed with water, the active ingredient will not settle out of solution or separate.

Solvent: A substance (usually liquid) suitable for, or employed in solution, or in dissolving something. For example, water is an appropriate solvent of most salts; alcohol of resins; ether of fats; and mercury or acids of metals.

Spreader: An adjuvant that allows an herbicide to form a uniform coating layer over the treated surface.

Stabilizer: A substance that renders or maintains a solution, mixture, suspension, or state resistant to chemical change.

Sticker: An adjuvant that causes an herbicide to adhere to plant foliage. Stickers reduce spray runoff during application and washoff by rain. Many stickers are blended with wetting agents so that they both increase spray coverage and provide better adhesion. These combined products often are call spreader-stickers.

Surfactant: A type of adjuvant designed to improve the dispersing/emulsifying, absorbing, spreading, sticking and/or pest-penetrating properties of an herbicide formulation. Surfactants can be divided into the following five groupings: 1) non-ionic surfactants, 2) crop oil concentrates, 3) nitrogen-surfactant blends, 4) esterified seed oils, and 5) organo-silicones.

Suitable surfactants may be nonionic, cationic, or anionic, depending on the nature of the compound used as an active ingredient Surfactants may be mixed together in some embodiments of the disclosure. Nonionic surfactants include polyglycol ether derivatives of aliphatic or cycloaliphatic alcohols, saturated or unsaturated fatty acids and alkylphenols. Fatty acid esters of polyoxyethylene sorbitan, such as polyoxyethylene sorbitan trioleate, also are suitable nonionic surfactants. Other suitable nonionic surfactants include water-soluble polyadducts of polyethylene oxide with polypropylene glycol, ethylenediaminopolypropylene glycol and alkylpolypropylene glycol. Particular nonionic surfactants include nonylphenol polyethoxyethanols, polyethoxylated castor oil, polyadducts of polypropylene and polyethylene oxide, tributylphenol polyethoxylate, polyethylene glycol and octylphenol polyethoxylate.

Cationic surfactants include quaternary ammonium salts carrying, as N-substituents, an 8 to 22 carbon straight or branched chain alkyl radical. The quaternary ammonium salts carrying may include additional substituents, such as unsubstituted or halogenated lower alkyl benzyl, or hydroxy-lower alkyl radicals. Some such salts exist in the form of halides, methyl sulfates, and ethyl sulfates. Particular salts include stearyldimethylammonium chloride and benzyl bis(2-chloroethyl)ethylammonium bromide.

Suitable anionic surfactants may be water-soluble soaps as well as water-soluble synthetic surface-active compounds. Suitable soaps include alkali metal salts, alkaline earth metal salts, and unsubstituted or substituted ammonium salts of higher fatty acids. Particular soaps include the sodium or potassium salts of oleic or stearic acid, or of natural fatty acid mixtures. Synthetic anionic surfactants include fatty sulfonates, fatty sulfates, sulfonated benzimidazole derivatives, and alkylarylsulfonates. Particular synthetic anionic surfactants include the sodium or calcium salt of ligninsulfonic acid, of dodecyl sulfate, or of a mixture of fatty alcohol sulfates obtained from natural fatty acids. Additional examples include alkylarylsulfonates, such as sodium or calcium salts of dodecylbenzenesulfonic acid, or dibutylnaphthalenesulfonic acid. Corresponding phosphates for such anionic surfactants are also suitable.

Tall Fescue (Festuca arundinacea): Tall fescue is a deep rooted, cool season perennial grass. The plant produces vigorous growth in the spring and fall and its extensive root system helps it withstand drought conditions. Tall fescue does produce short rhizomes but has a bunch-type growth habit; it spreads primarily by erect tillers. Individual tillers, or sterns, terminate in an inflorescence, reach about three to four feet in height, and have broad, dark green basal leaves. Leaf blades are glossy on the underside and serrated on the margins. The leaf sheath is smooth and the ligule is a short membrane. The inflorescence is a compact panicle, about three to four inches long with lanceolate spikelets about one-half inch or more long. The grass flowers in the spring and seed mature in early summer.

Tall fescue is found from the Pacific Northwest to the southern states in low-lying pastures. Although it grows best in moist environments, tall fescue has good drought tolerance and will survive during dry periods in a dormant state. Tall fescue is adapted to a wide range of soils, but does best on clay soils high in organic matter. Tall fescue is well adapted to the “transition zone” of the United States where summers are too hot and humid for cool season grasses and winters too cold for warm season grasses. In the South, tall fescue is best adapted to those states in the transition zone: Oklahoma, Arkansas, Missouri, Tennessee, Kentucky, Virginia and northern parts of North Carolina, Georgia and Texas.

Thickener: An adjuvant that reduces drift by increasing droplet size and reducing volume of spray contained in drift-prone droplets.

Timed-Release Coating: A coating on a solid or particulate GAF formulation that retards degradation and prolongs GAF activity. Coatings can be divided into three categories: (1) coatings that directly degrade in the presence of water, (2) coatings that are broken apart by wet and dry cycles, and (3) coatings degraded by specific temperatures, for example Degree Herbicides by Monsanto.

Vector: A nucleic acid molecule as introduced into a host cell, thereby producing a transfected host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

Water-Resistant Coating: A coating on a solid or particulate GAF formulation that repels water and delays dissolution of the herbicide. One common technique used commercially is interfacial polycondensation of multifunctional isocyanates with multifunctional amines. In this technique, the oil phase containing the active agene and the isocyanate is emulsified in the aqueous phase containing the amine monomer. The isocyanate reacts with the amine at the oil-water interface to form a solid polyurea shell wall about the encapsulated active agent. A second technique involves coating GAF with whey protein that has been treated to provide a specific form or structure that is more highly resistant to dissolution in water.

Weed: Any unsightly, useless, or injurious plant, particularly plants growing in cultivated ground to the injury of the crop or desired vegetation, plant seeds contaminating a desired seed variety, or plants growing to the disfigurement of an area, for example a garden or a road-side. The term “weed” has no definite application to any particular plant, or species of plants. In some embodiments, a weed is a grassy weed, for instance, annual bluegrass, downy brome, jointed goatgrass, roughstalk bluegrass, rattail fescue, perennial ryegrass, tall fescue, or domestic or wild oats.

Wettable powder: A dry, finely ground herbicide formulation in which the active ingredient is combined with a finely ground carrier (usually mineral clay), along with other ingredients to enhance the ability of the powder to suspend in water. Generally, the powder is mixed with water for application. Wettable powders are one of the most widely used herbicide formulations and offer low cost and ease of storage, transport, and handling; lower phytotoxicity potential than emulsifiable concentrates and other liquid formulations; and less skin and eye absorption hazard than emulsifiable concentrates and other liquid formulations. Some disadvantages are that they require constant and thorough agitation in the spray tank, are abrasive to pumps and nozzles (causing premature wear), may produce visible residues on plant and soil surfaces, and can create an inhalation hazard to the applicator while handling (pouring and mixing) the concentrated powder. Typical solid diluents are described in Watkins et al., Handbook of Insecticide Dust Diluents and Carriers, 2nd Ed., Dorland Books, Caldwell, N.J. The more absorptive diluents are preferred for wettable powders and the denser ones for dusts.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In case of conflict, the present specification, including explanations of terms, will control. In addition, the materials, methods, and examples are illustrative only and not intended to be limiting.

### III. Description of Several Embodiments

Disclosed herein are isolated bacterial strains, all of which have been deposited with the Agricultural Research Service (ARS) Culture Collection. The strains include Pseudomonas fluorescens Biotype B E34 (ARS #B-30481), Pseudomonas fluorescens Biotype C WH19 (ARS #B-30484), Pseudomonas fluorescens Biotype C WH6 (ARS #B-30485), Pseudomonas putida Biotype B AH4 (ARS #B-30482), and Pseudomonas putida Biotype B AD31 (ARS #B-30483). These bacterial strains inhibit or arrest grassy weed germination.

Also disclosed herein is a Germination-Arrest Factor. The factor is produced by Pseudomonas fluorescens Biotype B E34, Pseudomonas fluorescens Biotype C WH19, Pseudomonas fluorescens C Biotype WH6, Pseudomonas putida Biotype B AH4, or Pseudomonas putida Biotype B AD31, and it inhibits or arrests grassy weed germination. In some examples, the grassy weed is Poa annua, Poa trivialis or Bromus tectorum, and in some examples, the grassy weed is crabgrass, goosegrass, dallisgrass, bahiagrass, annual bluegrass, downy brome, jointed goatgrass, roughstalk bluegrass, rattail fescue, perennial rye grass, or tall fescue.

In certain examples, the Germination-Arrest Factor is a hydrophilic molecule, and in particular examples, the Germination-Arrest Factor has a molecular weight less than 3,000 daltons. In certain other examples, the Germination-Arrest Factor reacts with ninhydrin, and in other particular examples, the Germination-Arrest Factor includes an ionizable group.

Also described herein is an isolated nucleic acid as set forth in (a) SEQ ID NO: 2, (b) SEQ ID NO: 7, (c) SEQ ID NO: 10, or (d) sequences having at least 90% sequence identity to (a), (b), or (c), wherein the nucleic acid encodes a Germination-Arrest Factor or a protein involved in the synthesis and or secretion of a Germination-Arrest Factor.

Another embodiment of the disclosure is an isolated Germination-Arrest Factor protein that includes an amino acid sequence as set forth in (a) SEQ ID NO: 3, (b) SEQ ID NO: 4, (c) SEQ ID NO: 8, (d) SEQ ID NO: 11, (e) SEQ ID NO: 12, (f) SEQ ID NO: 13, (g) sequences having at least 90% sequence identity to (a), (b), (c), (d), (e), or (t), or (h) conservative variants of (a), (b), (c), (d), (e), or (t). The Germination-Arrest Factor protein inhibits or arrests germination in grassy weeds or is required for the synthesis and or secretion of a Germination-Arrest Factor that inhibits or arrests germination in grassy weeds. In some examples, the grassy weed is Poa annua, Poa trivialis or Bromus tectorum, and in some examples, the grassy weed is, annual bluegrass, downy brome, jointed goatgrass, roughstalk bluegrass, rattail fescue, and cultivated perennial rye grass, or tall fescue.

Also disclosed herein is a method of inhibiting or arresting weed germination in a growth medium in which it would be desirable to inhibit or arrest grassy weed germination. The method includes applying Pseudomonas fluorescens Biotype B E34, Pseudomonas fluorescens Biotype C WH19, Pseudomonas fluorescens C Biotype WH6, Pseudomonas putida Biotype B AH4, or Pseudomonas putida Biotype B AD31 to the growth medium in an amount sufficient to inhibit or arrest grassy weed germination.

Other embodiments of the disclosure are methods of inhibiting or arresting weed germination in a growth medium in which it would be desirable to inhibit or arrest grassy weed germination. The methods include applying the Germination-Arrest Factor to the growth medium in an amount sufficient to inhibit or arrest grassy weed germination. In some examples, the Germination-Arrest Factor is applied in a formulation that also includes a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide. In certain examples, the Germination-Arrest Factor is applied in a formulation that is a solution, a soluble powder, an emulsifiable concentrate, a wettable powder, a liquid flowable, a dry flowable, a water-dispersible granule, a granule, or a pellet.

Still other embodiments of the disclosure are methods of inhibiting or arresting weed germination in grass seed. The methods include applying Pseudomonas fluorescens Biotype B E34, Pseudomonas fluorescens Biotype C WH19, Pseudomonas fluorescens C Biotype WH6, Pseudomonas putida Biotype B AH4, or Pseudomonas putida Biotype B AD31, or mixtures thereof to the grass seed in an amount sufficient to inhibit or arrest grassy weed germination.

Also disclosed herein is a method of inhibiting or arresting weed germination in grass seed. The method includes applying Germination-Arrest Factor to the grass seed in an amount sufficient to inhibit or arrest grassy weed germination. In some examples, the Germination-Arrest Factor is applied in a formulation that also includes a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide. In certain examples, the Germination-Arrest Factor is applied in a formulation that is a solution, a soluble powder, an emulsifiable concentrate, a wettable powder, a liquid flowable, a dry flowable, a water-dispersible granule, a granule, or a pellet.

A further embodiment of the disclosure is a composition for inhibiting or arresting the germination of grassy weeds. The composition includes a Germination-Arrest Factor and a timed-release coating over at least a portion of the Germination-Arrest Factor. In certain examples, the composition also includes a water-resistant coating over the timed-release coating.

Still further embodiments are methods of inhibiting or arresting weed germination in a grass patch. The methods include broadcasting an herbicidally effective amount of a Germination-Arrest Factor at least once a year across a grass field in which inhibiting or arresting weed germination is desirable, thereby inhibiting or arresting weed germination. In some examples, the Germination-Arrest Factor is applied in a formulation that also includes a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide. In certain examples, the Germination-Arrest Factor is applied in a formulation that is a solution, a soluble powder, an emulsifiable concentrate, a wettable powder, a liquid flowable, a dry flowable, a water-dispersible granule, a granule, or a pellet. In certain examples, the Germination-Arrest Factor is formulated as a granule. In particular examples, the granule is at least partially coated with a timed-release coating, and in still more particular examples, the timed-release coating is coated with a water-resistant coating.

Also disclosed are methods of producing a Germination-Arrest Factor. The methods include culturing Pseudomonas fluorescens Biotype B E34, Pseudomonas fluorescens Biotype C WH19, Pseudomonas fluorescens C Biotype WH6, Pseudomonas putida Biotype B AH4, or Pseudomonas putida Biotype B AD31 in a suitable culture medium, collecting the culture medium, and purifying the culture medium to produce the Germination-Arrest Factor.

Yet another embodiment of the disclosure is a kit for inhibiting or arresting grassy weed growth. The kit includes a Germination-Arrest Factor, a container, and, optionally, instructions for using the kit.

Yet still other embodiments are Pseudomonas fluorescens or Pseudomonas putida bacterial strains having the GAF-producing characteristics of Pseudomonas fluorescens Biotype B E34 (deposited as NRRL # B-30481), Pseudomonas fluorescens Biotype C WH19 (deposited as NRRL # B-30484), Pseudomonas fluorescens C Biotype WH6 (deposited as NRRL # B-30485), Pseudomonas putida Biotype B AH4 (deposited as NRRL # B-30482), or Pseudomonas putida Biotype B AD31 (deposited as NRRL # B-30483). In certain embodiments, the Germination-Arrest Factor produced by these bacterial strains is a hydrophilic molecule, has a molecular weight less than 3,000 daltons, reacts with ninhydrin, and comprises an ionizable group.

### IV. Bacterial Strains

Disclosed herein are isolated bacterial strains, all of which have been deposited with the Agricultural Research Service (ARS) Culture Collection (NRRL). The strains include Pseudomonas fluorescens Biotype B E34 (NRRL #B-30481), Pseudomonas fluorescens Biotype C WH19 (NRRL #B-30484), Pseudomonas fluorescens Biotype C WH6 (NRRL #B-30485), Pseudomonas putida Biotype B AH4 (NRRL #B-30482), and Pseudomonas putida Biotype B AD31 (NRRL #B-30483). These bacterial strains inhibit or arrest grassy weed germination by producing Germination-Arrest Factor (see section V, below).

Other GAF-producing Pseudomonas strains also are readily identified that share the GAF-producing characteristics of deposited strains Pseudomonas fluorescens Biotype B E34 (NRRL #B-30481), Pseudomonas fluorescens Biotype C WH19 (NRRL #B-30484), Pseudomonas fluorescens Biotype C WH6 (NRRL #B-30485), Pseudomonas putida Biotype B AH4 (NRRL #B-30482), and Pseudomonas putida Biotype B AD31 (NRRL #B-30483). Such a bacterial strain is identified by screening the bacteria or bacterial products for GAF activity, for example using any of the seed germination assays described herein (see section VIII, below). Such a GAF-producing strain (or the bacterial product of such a strain), for example, irreversibly arrests the germination of the seeds of a number of grassy weeds, for example, but not limited to, annual bluegrass, downy brome, jointed goatgrass, roughstalk bluegrass, and rattail fescue, perennial rye grass, and tall fescue (which, in some embodiments of the disclosure, might fall to the ground during harvest of these crop plants), in addition to both domestic and wild oats. However, such a GAF-producing strain (or the bacterial product of such a strain), does not arrest the germination of seeds of wheat, corn, or barley.

In addition, the GAF produced by such a bacterial strain is a hydrophilic molecule that is insoluble or sparingly soluble in organic solvents. In certain embodiments, the GAF produced by such a bacterial strain has a molecular weight less than 3,000, reacts with ninhydrin, and contains at least one ionizable group.

One specific, non-limiting method for screening for GAF-producing bacterial strains involves eliminating certain contaminant strains in a mixed culture using a restriction digest profile of the DNA fragments from one or more of strains Pseudomonas fluorescens Biotype B E34 (NRRL #B-30481), Pseudomonas fluorescens Biotype C WH19 (NRRL #B-30484), Pseudomonas fluorescens Biotype C WH6 (NRRL #B-30485), Pseudomonas putida Biotype B AH4 (NRRL #B-30482), and Pseudomonas putida Biotype B AD31 (NRRL #B-30483). In some embodiments, strains that do not produce GAF have a very different restriction digest profile than that of the known GAF-producing strains. Conversely, in some embodiments, strains that produce GAF have a restriction digest profile similar or identical to that of the known GAF-producing strains.

### V. Germination-Arrest Factor

Disclosed herein is a highly specific, effective, naturally occurring bioherbicide that is an alternative to chemical herbicides (e.g., Diuron) for the control of grassy weeds. For example, in one embodiment of the disclosure, GAF is used in grass seed production systems or other cropping systems. In other embodiments of the disclosure, GAF is used in natural settings, in horticultural landscapes, or in other settings where grassy weeds are problematic.

GAF was identified as the product of particular isolates of certain rhizobacteria (Pseudomonas species; see Example 2) obtained from Willamette Valley soils in Oregon. GAF irreversibly arrests the germination of the seeds of a number of grassy weeds, for example, but not limited to, annual bluegrass, downy brome, jointed goatgrass, roughstalk bluegrass, and rattail fescue, perennial rye grass, and tall fescue (which, in some embodiments of the disclosure, might fall to the ground during harvest of these crop plants), in addition to both domestic and wild oats. GAF does not arrest the germination of seeds of wheat, corn, barley, or the dicot species tested to date.

GAF has a unique mode of action that distinguishes it from less selective inhibitors of seed germination. Sensitive seeds initiate germination in the presence of GAF, but seedling development is irreversibly arrested immediately following emergence of the coleorhiza and plumule. Thus, sensitivity to GAF is both species-specific and developmentally specific. Established seedlings appear to have little if any sensitivity to GAF, offering unique opportunities for weed management in grass cropping systems. Furthermore, culture filtrates containing GAF can be diluted, for example, filtrates can be diluted two- or three-fold, or even ten-fold, or more, with little or no loss of activity. Thus, GAF is active at very low absolute concentrations.

Three WH6 genes have been identified and sequenced that are essential for the production of GAF. Two of these have homology to bacterial genes of known function (one involved with secretion and the other with a minor chemical modification). The third gene encodes a putative polypeptide that has homology with a recent entry from the P. fluorescens Microbial Genomes Annotation Project This predicted polypeptide has a motif suggesting that it has serine/threonine kinase activity. These findings, as well as the developmental and species selectivity of GAF, indicate that GAF is a bacterial product that has not previously been described or identified.

GAF is a hydrophilic molecule that is insoluble or sparingly soluble in organic solvents. GAF has a molecular weight less than 3,000, it reacts with ninhydrin, and it contains at least one ionizable group. GAF chromatographs with defined Rf values in particular thin-layer chromatography systems, and mutation of any of the three genes that have been identified as essential for GAF biosynthesis results in both loss of the biological activity associated with GAF and loss of one particular ninhydrin-positive band visible in these TLC separations. Restoration (complementation) of the functional gene in the mutant bacteria results in the recovery of both biological activity and this particular ninhyrin-positive band, establishing that this compound is the one responsible for GAF activity.

An “effective amount” of GAF refers to an amount that has an adverse biological effect on at least some of the seeds exposed to the GAF. For example, the effective amount of a GAF may be an amount sufficient to arrest germination of at least some seeds in a seed population. In specific embodiments of the disclosure, an effective amount of a GAF arrests germination in at least 10% of the seeds treated. In particular embodiments of the disclosure, an effective amount of a GAF arrests germination in at least 20%, or even 50%, of a seed population. In more particular embodiments of the disclosure, an effective amount of a GAF arrests germination in over 90%, or nearly 100% of a seed population. Specific examples of effective amounts of GAF are provided in the Examples below.

### VI. Variation of Germination Arrest Factor Peptides, Germination-Arrest Factor Nucleic Acid Sequences, and Germination-Arrest Factor Molecules

A. Sequence Variants

The germination-arresting or -inhibiting characteristics of the peptides disclosed herein lie not in the precise amino acid sequence, but rather in the three-dimensional structure inherent in the amino acid sequences encoded by the DNA sequences. It is possible to recreate the binding characteristics of any of these peptides, for instance the germination-arresting or -inhibiting activity, by recreating the three-dimensional structure, without necessarily recreating the exact amino acid sequence. This can be achieved by designing a nucleic acid sequence that encodes for the three-dimensional structure, but which differs, for instance by reason of the redundancy of the genetic code. Similarly, the DNA sequence may also be varied, while still producing a functional herbicidal peptide.

Variant germination-arresting or -inhibiting peptides, and peptides that are required for the synthesis and/or secretion of Germination Arrest Factor, include peptides that differ in amino acid sequence from the disclosed sequences, but that share structurally significant sequence homology with any of the provided peptides. Such variants may be produced by manipulating the nucleotide sequence of the encoding sequence, using standard procedures, including site-directed mutagenesis or PCR. The simplest modifications involve the substitution of one or more amino acids for amino acids having similar biochemical properties. These so-called conservative substitutions are likely to have minimal impact on the activity of the resultant peptide, especially when made outside of the binding site of the peptide. Table 1 shows amino acids that may be substituted for an original amino acid in a peptide, and which are regarded as conservative substitutions.

More substantial changes in peptide structure may be obtained by selecting amino acid substitutions that are less conservative than those listed in Table 1. Such changes include changing residues that differ more significantly in their effect on maintaining polypeptide backbone structure (e.g., sheet or helical conformation) near the substitution, charge or hydrophobicity of the molecule at the target site, or bulk of a specific side chain. The following substitutions are generally expected to produce the greatest changes in protein properties: (a) a hydrophilic residue (e.g., seryl or threonyl) is substituted for (or by) a hydrophobic residue (e.g., leucyl, isoleucyl, phenylalanyl, valyl or alanyl); (b) a cysteine or proline is substituted for (or by) any other residue; (c) a residue having an electropositive side chain (e.g., lysyl, arginyl, or histadyl) is substituted for (or by) an electronegative residue (e.g., glutamyl or aspartyl); or (d) a residue having a bulky side chain (e.g., phenylalanine) is substituted for (or by) one lacking a side chain (e.g., glycine).

TABLE 1

| Original | Conservative |
|---|---|
| Residue | Substitutions |
| Ala | ser |
| Arg | lys |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

Variant Germination-Arrest Factor-encoding sequences, and sequences that code for peptides that are required for the synthesis and/or secretion of Germination Arrest Factor, may be produced by standard DNA mutagenesis techniques, for example, M13 primer mutagenesis. Details of these techniques are provided in Sambrook (In Molecular Cloning: A Laboratory Manual, Cold Spring Harbor, N.Y., 1989), Ch. 15. By the use of such techniques, variants may be created which differ in minor ways from the disclosed sequences. DNA molecules and nucleotide sequences which are derivatives of those specifically disclosed herein and that differ from those disclosed by the deletion, addition, or substitution of nucleotides while still encoding a peptide that inhibits or arrests germination or is required for the synthesis and/or secretion of a Germination Arrest Factor, are comprehended by this disclosure. In their most simple form, such variants may differ from the disclosed sequences by alteration of the coding region to fit the codon usage bias of the particular organism into which the molecule is to be introduced.

Alternatively, the coding region may be altered by taking advantage of the degeneracy of the genetic code to alter the coding sequence such that, while the nucleotide sequence is substantially altered, it nevertheless encodes a peptide having an amino acid sequence substantially similar to the disclosed fusion sequences. For example, the ninth amino acid residue of SEQ ID NO: 3 is alanine. Because of the degeneracy of the genetic code, four nucleotide codon triplets—(GCG, GCC, GCr and GCA)—code for alanine. Thus, the nucleotide sequence of the sequence could be changed at this position to any of these alternative codons without affecting the amino acid composition or characteristics of the encoded peptide. Based upon the degeneracy of the genetic code, variant DNA molecules may be derived from the cDNA and gene sequences disclosed herein using standard DNA mutagenesis techniques as described above, or by synthesis of DNA sequences. Thus, this disclosure also encompasses nucleic acid sequences that encode Germination-Arrest Factor peptides, and peptides that are required for the synthesis and/or secretion of Germination Arrest Factor, but which vary from the disclosed nucleic acid sequences by virtue of the degeneracy of the genetic code.

In some embodiments of the disclosure, a variant GAF sequence has, for example 70%, 75%, 80%, 85%, 90%, 95%, 98%, or 99% sequence identity with SEQ ID NO: 2, SEQ ID NO: 7, or SEQ ID NO: 10.

B. Peptide Modifications

The present disclosure includes biologically active molecules that mimic the action of the GAF peptides of the present disclosure. The peptides of the disclosure include synthetic embodiments of naturally-occurring peptides described herein, as well as analogues (non-peptide organic molecules), derivatives (chemically functionalized protein molecules obtained starting with the disclosed peptide sequences) and variants (homologs) of the GAF peptides. Each peptide of the disclosure is comprised of a sequence of amino acids, which may be either L- and/or D-amino acids, naturally occurring and otherwise.

Peptides may be modified by a variety of chemical techniques to produce derivatives having essentially the same activity as the unmodified peptides, and optionally having other desirable properties. For example, carboxylic acid groups of the peptides, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptides, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are also within the scope of the present disclosure, whereby the three-dimensional arrangement of the chemical constituents of such peptido- and organomimetics mimic the three-dimensional arrangement of the peptide backbone and component amino acid side chains in the GAF peptides, resulting in such peptido- and organomimetics of the peptides of this disclosure having measurable or enhanced germination-arresting activity. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology Munson (ed.) 1995, Ch. 102, for descriptions of techniques used in CADD. Also included within the scope of the disclosure are mimetics prepared using such techniques that produce germination-arresting peptides.

In some embodiments of the disclosure, a variant GAF peptide sequence has, for example 70%, 75%, 80%, 85%, 90%, 95%, 98%, or 99% sequence identity with SEQ ID NO: 3, SEQ ID NO: 4, SEQ ID NO: 8, SEQ ID NO: 11, or SEQ ID NO: 12.

C. Germination-Arrest Factor Molecules

The present disclosure includes biologically active molecules that mimic the action of the Germination-Arrest Factors of the present disclosure. The Germination-Arrest Factors of the disclosure include synthetic embodiments of the naturally-occurring compounds described herein, as well as analogues, derivatives and the variants of these compounds that specifically inhibit or arrest germination of grassy weeds.

GAF molecules may be modified by a variety of chemical and enzymatic techniques to produce derivatives having essentially the same activity as the unmodified GAF molecules and optionally having other desirable properties, such as changes in target specificity or enhanced efficacy. For example, various substituents may be introduced at the ninhydrin-reactive group or at the ionizable group known to be present on GAF molecules.

### VII. Production of Pseudomonas Extracts

GAF is produced by particular isolates of certain rhizobacteria (Pseudomonas species; see Example 2). In general Pseudomonas isolates are inoculated into Pseudomonas Minimal Salts Medium and incubated with gentle agitation for several days prior to harvest. The cultures are then centrifuged, and the supernatant is filtered.

Measured volumes of the culture filtrate are dried, and the dry solids recovered from the culture filtrate are extracted with ethanol or other solvents. The dry solids are then redissolved in a solvent appropriate to the planned experiment prior to use.

### VII. Seed Germination Assays

Any seed germination assay can be used to measure GAF activity. One such assay is the Standard GAF Bioassay System described herein. In the Standard GAF Bioassay System, aliquots of each test solution are distributed in different wells of a sterile multi-well culture plate. Surface-sterilized seeds are aseptically transferred aseptically to each well and submerged in the test solution. The culture plate is then sealed and incubated in a germinator for several days. Seed germination is scored on a four-point scale (see Table 5).

Seed germination also can be determined using a germination assay on filter paper. Surface sterilized seeds are vacuum dried and then sprinkled on glass-microfiber filter discs wetted with sterilized water and placed in a Petri dish. The Petri dishes are sealed with Parafilm, packaged in aluminum foil and put in a growth chamber for a preconditioning period.

After the preconditioning period, Petri dishes are opened and individual seeds are placed on individual glass-microfiber discs treated with sterile water or test solution. All Petri dishes are then resealed with Parafilm and returned to the growth chamber. Several days later, the seed germination rate is determined under a stereoscopic microscope by counting the number of seeds with and without an emerged radical.

Any other seed germination assay known in the art can be used to screen for OAF activity, for example assays outlined in the Ecological Effects Test Guidelines: OPPTS 850.4225 Seedling Emergence Tier II, Guidelines developed by the Office of Prevention, Pesticides and Toxic Substances, United States Environmental Protection Agency (available electronically from EPA Public Access Gopher (gopher.epa.gov).]

### IX. GAF Formulations

The compounds of the present disclosure can be combined with appropriate solvents or surfactants to form a product called a formulation. Formulations enable the uniform distribution of a relatively small amount of GAF over a comparatively large area. In addition to providing the user with a form of GAF that is easy to handle, formulating GAF can enhance its phytotoxicity, improve its shelf-life, and protect it from adverse environmental conditions while in storage or transit.

The primary kinds of herbicide formulations are: solutions, soluble powders, emulsifiable concentrates, wettable powders, liquid flowables, dry flowables, water-dispersible granules, granules, and pellets. Formulations vary according to the solubility of the herbicide active ingredient in water, oil and organic solvents, and the manner the formulation is applied (i.e., dispersed in a carrier, such as water, or applied as a dry formulation).

Solution formulations are designed for those active ingredients that dissolve readily in water. The formulation is a liquid and consists of the active ingredient and additives. Generally, when herbicides formulated as solutions are mixed with water, the active ingredient will not settle out of solution or separate. Suitable liquid carriers, such as solvents, may be organic or inorganic. Water is one example of an inorganic liquid carrier. Organic liquid carriers include vegetable oils and epoxidized vegetable oils, such as rape seed oil, castor oil, coconut oil, soybean oil and epoxidized rape seed oil, epoxidized castor oil, epoxidized coconut oil, epoxidized soybean oil, and other essential oils. Other organic liquid carriers include silicone oils, aromatic hydrocarbons, and partially hydrogenated aromatic hydrocarbons, such as alkylbenzenes containing 8 to 12 carbon atoms, including xylene mixtures, alkylated naphthalenes, or tetrahydronaphthalene. Aliphatic or cycloaliphatic hydrocarbons, such as paraffins or cyclohexane, and alcohols, such as ethanol, propanol or butanol, also are suitable organic carriers. Gums, resins, and rosins used in forest products applications and naval stores (and their derivatives) also may be used. Additionally, glycols, including ethers and esters, such as propylene glycol, dipropylene glycol ether, diethylene glycol 2-methoxyethanol, and 2-ethoxyethanol, and ketones, such as cyclohexanone, isophorone, and diacetone alcohol may be used. Strongly polar organic solvents include N-methylpyrrolid-2-one, dimethyl sulfoxide, and N,N-dimethylformamide.

Typical liquid diluents and solvents are described in Marsden, Solvents Guide, 2nd Ed., Interscience, NY, 1950. Solubility under 0.1% is preferred for suspension concentrates; solution concentrates are preferably stable against phase separation at 0° C. McCutcheon's Detergents and Emulsifiers Annual, Allured Publ. Corp., Ridgewood, N.J., as well as Sisely and Wood, Encyclopedia of Surface Active Agenits, Chemical Publ., Co., Inc., NY 1964, list surfactants and recommended uses.

Soluble powder formulations are similar to solutions in that, when mixed with water, they dissolve readily and form a true solution. Soluble powder formulations are dry and include the active ingredient and additives. When thoroughly mixed, no further agitation is necessary to keep the active ingredient dissolved in solution.

Emulsifiable concentrate formulations are liquids that contain the active ingredient, one or more solvents, and an emulsifier that allows mixing with water. Formulations of this type are highly concentrated, relatively inexpensive per pound of active ingredient, and easy to handle, transport, and store. In addition, they require little agitation (will not settle out or separate) and are not abrasive to machinery or spraying equipment Formulations of this type may, however, have greater phytotoxicity than other formulations, and they are subject to over- or underdosing through mixing or calibration errors. In addition, these types of formulations are more easily absorbed through skin of humans or animals, and contain solvents that may cause deterioration of rubber or plastic hoses and pump parts.

Wettable powders are dry, finely ground formulations in which the active ingredient is combined with a finely ground carrier (usually mineral clay), along with other ingredients to enhance the ability of the powder to suspend in water. Generally, the powder is mixed with water for application. Wettable powders are some of the most widely used herbicide formulations and offer low cost and ease of storage, transport, and handling; lower phytotoxicity potential than emulsifiable concentrates and other liquid formulations; and less skin and eye absorption hazard than emulsifiable concentrates and other liquid formulations. Some disadvantages are that they require constant and thorough agitation in the spray tank, are abrasive to pumps and nozzles (causing premature wear), may produce visible residues on plant and soil surfaces, and can create an inhalation hazard to the applicator while handling (pouring and mixing) the concentrated powder. Typical solid diluents are described in Watkins et al., Handbook of Insecticide Dust Diluents and Carriers, 2nd Ed., Dorland Books, Caldwell, N.J. The more absorptive diluents are preferred for wettable powders and the denser ones for dusts.

Liquid flowable formulations are made up of finely ground active ingredient suspended in a liquid. Flowables generally are mixed with water for application, are easily handled and applied, and seldom clog nozzles. Some of their disadvantages are that they may leave a visible residue on plant and soil surfaces, and typically require constant and thorough agitation to remain in suspension.

Dry flowable and water-dispersible granule formulations are much like wettable powders except that the active ingredient is formulated on a large particle (granule) instead of onto a ground powder. This type of formulation offers essentially the same advantages and disadvantages as wettable powder formulations. However, these formulations generally are more easily mixed and measured than wettable powders. Because they create less dust when handling, they cause less inhalation hazard to the applicator during pouring and mixing.

Granules and pellets are used for soil-applied herbicides. In a granule or pellet formulation, the active ingredient is formulated onto large particles (granules or pellets). The primary advantages of this type of formulation are that the formulation is ready to use with simple application equipment (seeders or spreaders), and the drift potential is low because the particles are large and settle quickly. The disadvantages of these formulations are that they do not adhere to foliage (not intended for foliar applications), and may require mixing into the soil in order to achieve adequate herbicidal activity.

Granulated materials of inorganic or organic nature may be used in formulating granules and pellets, such as dolomite or pulverized plant residues. Suitable porous granulated adsorptive carriers include pumice, broken brick, sepiolite, and bentonite. Additionally, nonsorbent carriers, such as sand, may be used. Some solid carriers are biodegradable polymers, including biodegradable polymers that are digestible or degrade inside an animal's body over time.

The methods of making such formulations are well known. Solutions are prepared by simply mixing the ingredients. Fine, solid compositions are made by blending and, usually, grinding, as in a hammer or fluid energy mill. Suspensions are prepared by wet-milling (see, for example, U.S. Pat. No. 3,060,084). Granules and pellets may be made by spraying the active material upon preformed granular carriers or by agglomeration techniques. See J. E. Browning, “Agglomeration”, Chemical Engineering, Dec. 4, 1967, p 147, and Perry's Chemical Engineer's Handbook, 4th Ed., McGraw-Hill, NY, 1963, pp. 8-59. For further information regarding the art of formulation, see, for example: U.S. Pat. No. 3,235,361, U.S. Pat. No. 3,309,192, U.S. Pat. No. 2,891,855, Klingman, Weed Control as a Science, John Wiley & Sons, Inc., New York, 1961 pp. 81-96, and Fryer and Evans, Weed Control Handbook, 5th Edn. Blackwell Scientific Publications, Oxford, 1968, pp. 101-103.

In selecting a formulation for an herbicide, the following considerations may be weighed: 1) how the formulation will affect the phytotoxicity of undesirable plants and/or desirable plant, 2) how the formulation will influence the compatibility of other crop protection chemicals, 3) what application machinery are available and most suited for the job, 4) how the formulation will affect the life of the application equipment, 5) whether the application equipment is designed for applying a particular formulation, and 6) concerns about safety for the applicator and other people.

The concentration of a compound, such as GAF, which serves as an active ingredient, may vary according to particular compositions and applications. In some embodiments of the disclosure, the percentage by weight of the active ingredient will be from about 0.1% to about 90%, for example to give final GAF concentrations equivalent to, or greater than, that in Pseudomonas fluorescens WH6 culture filtrate. A suitable amount for a particular application may be determined using bioassays for the particular pest intended to be controlled. Higher concentrations are usually employed for commercial purposes or products during manufacture, shipment, or storage; such embodiments have concentrations at least about 10%, or from about 25% to about 90% by weight. Prior to use, a highly concentrated formulation may be diluted to a concentration appropriate for the intended use, such as from about 0.1% to 10%, or from about 1% to 5%.

### X. Inactive Ingredients in GAF Formulations

In some embodiments of the disclosure, inactive ingredients, (adjuvants) are added to herbicide formulations to improve the performance of the herbicide. For example, in one embodiment of the disclosure, GAF is formulated with a surfactant A surfactant (surface active agent) is a type of adjuvant designed to improve the dispersing/emulsifying, absorbing, spreading, sticking and/or pest-penetrating properties of the spray mixture. Surfactants can be divided into the following five groupings: 1) non-ionic surfactants, 2) crop oil concentrates, 3) nitrogen-surfactant blends, 4) esterified seed oils, and 5) organo-silicones.

Suitable surfactants may be nonionic, cationic, or anionic, depending on the nature of the compound used as an active ingredient. Surfactants may be mixed together in some embodiments of the disclosure. Nonionic surfactants include polyglycol ether derivatives of aliphatic or cycloaliphatic alcohols, saturated or unsaturated fatty acids and alkylphenols. Fatty acid esters of polyoxyethylene sorbitan, such as polyoxyethylene sorbitan trioleate, also are suitable nonionic surfactants. Other suitable nonionic surfactants include water-soluble polyadducts of polyethylene oxide with polypropylene glycol, ethylenediaminopolypropylene glycol and alkylpolypropylene glycol. Particular nonionic surfactants include nonylphenol polyethoxyethanols, polyethoxylated castor oil, polyadducts of polypropylene and polyethylene oxide, tnibutylphenol polyethoxylate, polyethylene glycol and octylphenol polyethoxylate. Cationic surfactants include quaternary ammonium salts carrying, as N-substituents, an 8 to 22 carbon straight or branched chain alkyl radical.

The quaternary ammonium salts carrying may include additional substituents, such as unsubstituted or halogenated lower alkyl, benzyl, or hydroxy-lower alkyl radicals. Some such salts exist in the form of halides, methyl sulfates, and ethyl sulfates. Particular salts include stearyldimethylammonium chloride and benzyl bis(2-chloroethyl)ethylanmuonium bromide.

Suitable anionic surfactants may be water-soluble soaps as well as water-soluble synthetic surface-active compounds. Suitable soaps include alkali metal salts, alkaline earth metal salts, and unsubstituted or substituted ammonium salts of higher fatty acids. Particular soaps include the sodium or potassium salts of oleic or stearic acid, or of natural fatty acid mixtures. Synthetic anionic surfactants include fatty sulfonates, fatty sulfates, sulfonated benzimidazole derivatives, and alkylarylsulfonates. Particular synthetic anionic surfactants include the sodium or calcium salt of ligninsulfonic acid, of dodecyl sulfate, or of a mixture of fatty alcohol sulfates obtained from natural fatty acids. Additional examples include alkylarylsulfonates, such as sodium or calcium salts of dodecylbenzenesulfonic acid, or dibutylnaphthalenesulfonic acid. Corresponding phosphates for such anionic surfactants are also suitable.

Other adjuvants include carriers and additives, for example, wetting agents, such as anionic, cationic, nonionic, and amphoteric surfactants, buffers, stabilizers, preservatives, antioxidants, extenders, solvents, emulsifiers, invert emulsifiers, spreaders, stickers, penetrants, foaming agents, anti-foaming agents, thickeners, safeners, compatibility agents, crop oil concentrates, viscosity regulators, binders, tackers, drift control agents, or other chemical agents, such as fertilizers, antibiotics, fungicides, nematicides, or pesticides. Such carriers and additives may be used in solid, liquid, gas, or gel form, depending on the embodiment and its intended application.

Additionally, the composition may include plural herbicidal compounds. Such a composition includes GAF as described herein and a second herbicidal compound.

### XI. Methods of Use

The GAF compounds disclosed herein may be used to control grassy weeds and cultivated grasses, for example, but not limited to, annual bluegrass, downy brome, jointed goatgrass, roughstalk bluegrass, and rattail fescue, perennial ryegrass, and tall fescue, in addition to both domestic and wild oats. In one embodiment of the disclosure, GAF is used in grass seed production systems or other cropping systems. In other embodiments of the disclosure, GAF is used in natural settings, horticultural landscapes, or any setting where grassy weeds are problematic. One specific, non-limiting example of a method of use of GAF is to control grassy weeds in natural settings, such as wilderness areas, lands under control of the Bureau of Land Management, or in ecologically sensitive areas. Another specific, non limiting example of a method of use is to control grassy weeds along roadsides. In any particular embodiment of the disclosure, GAF is applied in an herbicidally effective amount. That amount may depend on a variety of factors, including (but not limited to) the area to be treated, the seed or growth medium to be treated, or the mechanism of application.

In one embodiment of the disclosure, GAF is applied by direct application to the growth medium, for example soil. Soil-applied herbicides may be used at various times, including several weeks before crop planting (early preplant), just prior to crop planting (preplant or preplant-incorporated), immediately after crop planting but before weed emergence (preemergence), or after crop and generally weed emergence (postemergence), and directed onto the soil or weeds while preventing contact with the crop (post-directed). Activity of soil-applied herbicides is influenced by soil texture, organic matter content, pH, water status, and tillage. Herbicide uptake may occur via roots, seeds, shoots, and vegetative propagules.

In one embodiment of the disclosure, GAF is applied as an early preplant herbicide. Early preplant herbicides are applied to the soil weeks in advance of crop planting, so herbicides applied in this manner generally have sufficient residual activity in soils to extend through the first few weeks after emergence.

In another embodiment of the disclosure, GAF is applied as a preplant-incorporated herbicide. Generally, preplant-incorporated herbicides are physically mixed into the soil prior to crop planting. Incorporation usually is performed with some type of secondary tillage implement that distributes the herbicide evenly in the top two to three inches of the soil.

In another embodiment of the disclosure, GAF is applied as a preemergence herbicide. Preemergence herbicides generally are applied to the soil prior to crop and weed emergence. In annual crops, preemergence herbicides are usually applied immediately after the crop is planted. Herbicides applied to the soil in perennial crops, turf grass, pastures, or non-cropland areas before weed seedlings emerge are also considered preemergence herbicides. Rainfall or irrigation moves preemergence herbicides into the top few inches of soil for optimum activity.

In other embodiments of the disclosure, GAF is applied as a foliar-applied herbicide to control developing weed seeds. The effectiveness of foliar-applied herbicides is determined by the rate and timing of application, target weed species, use of spray adjuvants, application equipment, and environmental conditions. Foliar penetration of herbicides is greatest in non-stressed plants under conditions of moderate temperature and high relative humidity. A rain-free period following postemergence herbicide application is helpful to allow a toxic dosage of the herbicide to be absorbed by the target plants.

In other embodiments of the disclosure, GAF is applied as a seed-cleaning adjuvant in seed-cleaning processes as a supplement or alternative to physical removal of target weed seeds.

In some embodiments of the disclosure, GAF is applied once, while alternative embodiments of the disclosure employ plural applications of GAF. In particular embodiments of the disclosure, GAF is applied on a weekly, monthly, quarterly, or annual basis. In any particular embodiment of the disclosure, the frequency of application may be regular or irregular, and the time elapsed between successive applications may be the same or different. For example, and without limitation, GAF may be applied every other week; every other month; twice a month; every three months; every six months; every nine months; or annually. The frequency and number of applications of GAF may depend on a variety of factors, including (but not limited to) the area to be treated, the seed variety to be treated, environmental conditions, and the method of application.

In certain embodiments of the disclosure, GAF is applied in an area-wide manner, such as in protection of agricultural crops, for example grass or wheat crops. In addition to agricultural applications, area-wide applications may include silvicultural, horticultural, or other forms of environmental grassy weed management and control. In such embodiments of the disclosure, GAF may be applied to the soil, such as drenching a particular locus with a liquid formulation or applying the active ingredient in solid form to a locus.

Certain embodiments use GAF for grassy weed control in grass seed production and storage. For example, GAF may be applied to grass seed or with non-targeted crop seed (for example, but not limited to, clover, wheat, corn, barley) to arrest germination of grassy weeds. In some examples, GAF is applied to the seed by soaking, coating, or dressing seeds prior to sowing. In another example, GAF is applied to a physical substrate that serves as a germination barrier as in seed strips for landscape applications.

GAF also may be applied to the soil where the seeds will be planted, such as in-furrow or in-field application. In such applications, GAF may be applied to provide a certain concentration of the compound in the environment at a particular locus. That certain concentration may be measured, established, or determined according to the needs of the user. For example, when applying the GAF to crops, the rate of application may depend on the nature of soil, the type of application (e.g., spraying crop foliage, burial in soil), the crop plant to be protected, the weed species to be controlled, the prevailing climatic conditions, the growing season, and other factors.

As another example, when applying GAF to stored or transported agricultural products, the rate of application may depend on the localized environment (e.g., storage within a warehouse, storage under a covered shelter, transport within a trailer), expected duration of storage, product to be protected, the weed species to be controlled, economic considerations, and other factors. In certain embodiments, the rates of concentration are in the range from about 0.01 to about 1000 ppm (parts-per-million), such as from about 0.1 to about 500 ppm, of active ingredient. In area-wide applications, rates of application per hectare may be from about 0.5 g/ha to 2000 g/ha, such as particularly from about 10 to 1000 g/ha, or from about 20 to 600 g/ha.

### XII. GAF Kits

In some embodiments of the disclosure, GAF is embodied in an acceptable carrier and stored within a container capable of storing the composition for its shelf life. The container may be made of any suitable material such as plastic or other polymer, glass, metal, or the like. In some embodiments of the disclosure, printed instructions and/or a printed label indicating that the composition may be used to control grassy weeds are associated with this container. In certain examples, the instructions and/or label provides information regarding the use of the composition for herbicidal purposes, and is associated with the container by being adhered to the container, or accompanying the container in a package. In particular examples, the instructions specify the weeds intended to be controlled by the composition, the method and rate of application, dilution protocols, use precautions, and the like. Additionally, the container may include a feature or device for applying the composition to the seed population or locus to be treated. For example, if the article of manufacture includes a liquid composition, the feature or device may be a hand-operated, motorized, or pressurized pressure-driven sprayer.

In some embodiments, for large-scale applications, a GAF kit may include a drum, whereas for household kits for control of grassy weeds GAF may be provided in a can or bottle. In other embodiments, GAF is provided as small scale, highly purified material for experimental use in understanding plant developmental processes. In still other embodiments, GAF is provided as a seed cleaning adjuvant

### EXAMPLES

### Example 1

### General Methods

A. Growth of Bacterial Cultures

Pseudomonas isolates stored in cryovials (50% glycerol, −60° C.) were inoculated into Wheaton bottles half-filled with Pseudomonas Minimal Salts Medium (PMS). The tops of the bottles were loosely capped and secured with tape. The bottles were placed on a rotary shaker (200 rpm) in a 27° C. chamber and allowed to grow for 7 days prior to harvest

B. Pseudomonas Minimal Salts Medium (PMS)

The PMS medium used was developed by Gasson (Applied and Environmental Microbiology (1980). 39:25-29). The medium is made as follows: Dissolve 0.2 g potassium chloride, 1.0 g ammonium phosphate, 2.0 g sodium phosphate (monobasic), and 4.96 g sodium phosphate (dibasic) in distilled water to make 1 liter final volume. Autoclave. Allow the medium to cool and then add 2 ml of a sterile solution of 20% (w/v) magnesium sulfate (heptahydrate) and 20 ml of a sterile 10% (w/v) glucose solution per liter of autoclaved medium.

C. Preparation of Culture Filtrates

Pseudomonas isolates were grown in PMS medium (see above) for 7 days. The cultures were centrifuged (3,000×g, 15 minutes), and the supernatant was passed through a bacteriological filter (Millipore GP Express Steritop, 0.22 mM pore size). The resulting sterile culture filtrates were stored at 4° C.

D. Solvent Extraction of Dried Culture Filtrates

Measured volumes of culture filtrate from Pseudomonas fluorescens WH6 (or other bacterial strains as indicated in the text) were taken to dryness in vacuo at 45° C. The evaporation flask was selected to have a volume at least ten-times that of the aliquot of culture filtrate to be evaporated. The dry solids recovered from the culture filtrate were extracted three times (5 minutes per extraction) with 90% (v/v) ethanol (or other test solvents as indicated in the text). For each of these three extractions, the dry solids were swirled with a volume of solvent equal to one-third of the original volume of culture filtrate. The three extracts prepared in this manner were combined and either stored at 4° C. for later use or immediately taken to dryness in vacuo at 45° C. and redissolved in a solvent appropriate to the planned experiment.

E. Surface Sterilization of Seed

The seed of Poa annua (or other test seeds) were surface-sterilized by placing two to five grams of seeds in a 50 ml glass beaker, covering the seeds with a 46 to 48% (v/v) solution of H₂SO₄, and stirring vigorously for 5 minutes. The seed/acid mixture was filtered through a gouch crucible and repeatedly rinsed with tap water to remove residual acid. The seeds were then placed in a beaker and covered with bleach (NaOCl, 5.25%) containing 1% (v/v) polyoxethylene sorbitan monolaurate Tween-20). The seeds were stirred for 5 minutes and then repeatedly rinsed with sterile deionized water and placed in a sterile Petri dish. The seed can be used immediately while wet or dried (spread in a thin layer and allowed to dry under a laminar flow hood with the Petri dish lid ajar for 4 to 8 hours) to prevent premature germination.

F. Standard GAF Bioassay System

Bioassays for GAF activity were performed with Poa annua (annual bluegrass) seeds unless otherwise indicated. For each concentration of each test solution, 200 μl aliquots of solution were distributed to each of 3 wells of a sterile Costar forty-eight well tissue culture plate. Three surface-sterilized Poa annua seed were aseptically transferred to each well and submerged in the liquid, providing a total of nine observations for each concentration of each solution tested. The lid of the tissue culture plate was sealed with Parafilm, and the plate was placed in a germinator (20° C., 8 hour day length at a light intensity of 50 μmol/m²/s, 16 hour dark period) for seven days.

G. TLC Chromatography

Aliquots (75 ml each) of culture filtrates (from Pseudomonas fluorescens Wh6 wildtype cultures, from cultures of the GAF mutants of Wh6, and from cultures of the GAF mutants following complementation with the corresponding wildtype GAF gene) were each taken to dryness in vacuo at 45° C. The samples of dry solids recovered in this manner were each extracted three times with 90% (v/v) ethanol (25 ml for 5 minutes per each extraction of each sample). The combined ethanol extracts from a given sample were dried in vacuo (45° C.) and redissolved in 3.75 ml of 76% (v/v) ethanol to give a chromatographic sample of approximately 20-fold (20×) concentration relative to the original culture filtrate.

For TLC chromatography, 0.15 ml of the 20× concentrate was applied as a band across Avicel Microcrystalline Cellulose TLC plates (250 μm thick, 5×20 cm). The chromatograms were developed over a distance of 15 cm with ethyl acetate:isopropanol:water (15:30:20) as the developing solvent. The chromatographic separations were performed in cylindrical chromatography tanks (6 cm×23 cm) containing 25 ml of the developing solvent. The developed chromatograms were examined and photographed under short-wave UV light

For bioassay of GAF activity, the cellulose was scraped from the developed plates as 1×5 cm wide zones (as indicated). The cellulose from each zone was collected in a 2.0 ml Minifuge tube and suspended in 1.0 ml of distilled water by vortexing. The cellulose was then pelleted by centrifugation (15,000 rpm, Minifuge), and the supernatant from each tube was tested for GAF activity in the standard GAF bioassay system (see above) using seeds of annual bluegrass (Poa annua).

For ninhydrin staining, the developed TLC chromatograms were sprayed with a solution of 0.25% (w/v) ninhydrin dissolved in 100 ml of 95% (v/v) ethanol containing 3.0 ml of glacial acetic acid and then heated in an oven at 80-90° C. for 20 minutes.

### Example 2

### Selection of Pseudomonas Isolates that Arrest Germination of the Seeds of Grassy Weeds

Summary

Pseudomonas bacteria were isolated from the rhizosphere of Poa, Triticale, Triticum, Hordeum, and Lolium species. These bacterial isolates were screened initially for the ability of live cultures to cause stunting of the roots and shoots of young seedlings of the grassy weed known as annual bluegrass (Poa annua). Isolates selected in this manner were evaluated further by testing the ability of live cultures to arrest the germination of seeds of Poa annua. Twelve isolates were active in this test, and culture filtrates prepared from these isolates also were active in arresting the germination of the seeds of Poa annua. On the basis of the latter bioassay, these isolates were presumed to produce and secrete a Germination-Arrest Factor (GAF) into the culture medium. Five of the more active isolates (WH6, E34, AD31, AH4 and WH19) were selected for further study. These five isolates were taxonomically identified using Fatty Acid Methyl Ester Analysis and were deposited into the ARS Patent Culture Collection (NRRL). The five isolates identified in this manner were cultured, lyophilized, and submitted to the ARS Patent Culture Collection for deposit under the conditions of the Budapest Treaty on the International Recognition of the Deposit of Microorganisms for the Purpose of Patent Procedures on Jun. 13, 2001. Listed here are the strains and the corresponding NRRL numbers: E34, B-30481; AH4, B-30482; AD31, B-30483; WH19, B-30484; WH6, B-30485]

A. Isolation of Pseudomonas Bacteria from the Rhizosphere

Roots of Poa, Triticale, Triticum, Hordeum, and Lolium were collected from a variety of locations on agricultural landscapes and roadsides throughout the Willamette Valley and Coast Range Valleys of Western Oregon. Single colony forming units (SCFU) of Pseudomonas spp. were isolated from water-washes of roots collected from each site. Individual colonies that developed on Fluorescent Pseudomonas Medium (FPM; Sands and Rovira, Applied Microbiology (1970) 20:513-514; Simon et al., J. Applied Bacteriology (1973) 98:503-517) were transferred once on the same medium, and stored at 4° C. Each SCFU was screened for ability to stunt young seedlings of Poa annua by placing pre-germinated seedlings onto cooled agar containing 10% (v/v) of a live bacterial culture grown in Pseudomonas Minimal Salt Medium (PMS; Gasson, Applied and Environmental Microbiology (1980). 39:25-29; see General Methods, Example 1.) Twenty-nine isolates that reduced shoot and/or root elongation of the annual bluegrass seedlings by more than 20% when compared to controls were identified. These isolates were transferred to PMS, grown to an OD 5_{80 nm} of at least 1.0, and stored in 50% glycerol at −60° C.

B. Screening of Pseudomonas Isolates for Ability to Arrest the Germination of Seeds of Poa annua (Annual Bluegrass)

The Pseudomonas isolates selected by the screening procedures described above were tested for their ability to arrest the germination of Poa annua seeds. Surface sterilized seeds were sown on PMS agar that had been mixed with liquid cultures of the test isolate. Of the 29 isolates tested in this manner, eight did not affect germination (but did stunt subsequent elongation of the root and shoot of the seedlings), nine allowed partial germination to occur, and twelve suppressed germination. The twelve isolates that suppressed germination of Poa annua seeds in these live culture tests were selected for further study. The origins of these isolates are summarized below (Table 2). Seven of the twelve isolates were derived from the rhizosphere of Poa, three from Triticale, and two from Triticum.

TABLE 2
Origin of Pseudomonas isolates exhibiting ability to arrest the germination
of Poa annua (annual bluegrass) seeds in tests with live bacterial cultures

| Isolate | | |
|---|---|---|
| Code | Isolated From: | Collection Location: |
| AH10 | Healthy Poa spp. | Disturbed site, Alsea Valley, |
| | | Benton Co., OR |
| AH4 | Healthy Poa spp. | Disturbed site, Alsea Valley, |
| | | Benton Co., OR |
| AD31 | Dying Poa spp. | Cut bank, Alsea Valley, |
| | | Benton Co., OR |
| BT1 | Mixed Poa spp. | Research Plot, Botany Farm, OSU, |
| | | Linn Co., OR |
| E34 | Mixed Poa spp. | Hyslop Crops Research Farm, OSU, |
| | | Benton Co., OR |
| E24 | Mixed Poa spp. | Hyslop Crops Research Farm, OSU, |
| | | Benton Co. OR |
| TDH5 | Healthy Poa spp. | Field of organically grown vegetables, |
| | | Benton Co., OR |
| TR44 | Healthy Triticale | Research Plot, Botany Farm, OSU, |
| | | Linn Co., OR |
| TR46 | Healthy Triticale | Research Plot, Botany Farm, OSU, |
| | | Linn Co., OR |
| TR33 | Healthy Triticale | Research Plot, Botany Farm, OSU, |
| | | Linn Co., OR |
| WH19 | Healthy Triticum | Hyslop Crops Research Farm, OSU, |
| | | Benton Co., OR |
| WH6 | Healthy Triticum | Hyslop Crops Research Farm, OSU, |
| | | Benton Co., OR |

The twelve isolates indicated were selected on the basis of their ability to arrest germination of Poa annua seeds in tests using live bacterial cultures. These twelve isolates were selected from an initial group of 29 isolates exhibiting ability to cause stunting of Poa annua seedlings in tests with live cultures. The tests with live cultures were performed as described in Example 1.

C. Selection of Pseudomonas Isolates Releasing a Highly Active Germination-Arrest Factor (GAF) into the Culture Medium

Each of the twelve Pseudomonas isolates that arrested germination of Poa annua seeds in tests using live cultures were cultured in liquid PMS medium (see Example 1). Bacteria-free culture filtrates were prepared from these cultures as described in Example 1. The culture filtrates were diluted to 25% of their original concentration and tested for their ability to arrest the germination of Poa annua seeds. Culture filtrates from five of the twelve isolates tested maintained complete arrest of germination at this dilution (Table 3), indicating that a highly active Germination-Arrest Factor (GAF) was being released into the culture medium. These isolates (WH6, E34, AD31, AH4 and WH19) were selected for further study.

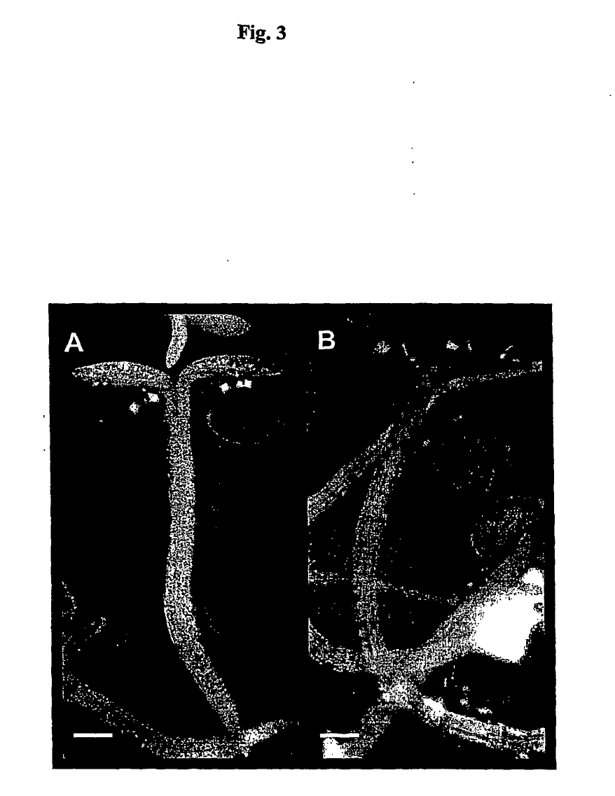

Experiments were also conducted to determine the effects of GAF on the germination of the seeds of dicotyledonous plants (Table 8). Germination of surface-sterilized seeds of red clover, white clover, and Arabidopsis placed on agar mixed with live WH6 bacterial cultures was unaffected by the presence of the bacteria. Similarly, treatment of Arabidopsis with WH6 culture filtrate and GAF extracts prepared from this source in ten-fold concentrates resulted in somewhat reduced elongation of the roots and shoots of Arabidopsis seedlings, but no effect on seed germination was observed (FIG. 3).

TABLE 3
Effects of culture filtrates from selected Pseudomonas isolates on the
germination of seeds of Poa annua (annual bluegrass)

| | OBSERVED GEMINATION RESPONSE | | |
|---|---|---|---|
| | AT RELATIVE SAMPLE CONCENTRATION (X) INDICATED | | |
| ISOLATE | 0.25X | 0.5X | 1.0X |
| AH10 | Stunting of seedling growth | Germination arrested | Germination arrested |
| AH4* | Germination arrested | Germination arrested | Germination arrested |
| AD31* | Germination arrested | Germination arrested | Germination arrested |
| BT1 | Normal development | Stunting of seedling | Germination arrested |
| | | growth | |
| E34* | Germination arrested | Germination arrested | Germination arrested |
| E24 | Stunting of seedling | Germination arrested | Germination arrested |
| | growth | | |
| TDH5 | Stunting of seedling | Germination arrested | Germination arrested |
| | growth | | |
| TR44 | Normal development | Stunting of seedling | Germination arrested |
| | | growth | |
| TR46 | Stunting of seedling | Germination arrested | Germination arrested |
| | growth | | |
| TR33 | Stunting of seedling | Germination arrested | Germination arrested |
| | growth | | |
| WH19* | Germination arrested | Germination arrested | Germination arrested |
| WH6* | Germination arrested | Germination arrested | Germination arrested |
| Culture filtrates were prepared from the indicated Pseudomonas isolates (identified as arresting the germination of seeds of Poa annua in preliminary screening using live bacteria). Culture filtrates were prepared as described in Example 1, diluted with sterile distilled water to give the | | | |
| # concentrations indicated below, and tested by the same procedures used in the Standard GAF Bioassay System (see Example 1). | | | |

*These isolates, which arrested germination at the lowest concentration tested (0.25X), were selected for further study.

D. Identification of Isolates and Deposit of Reference Cultures

The five Pseudomonas isolates selected for high GAF activity as described above were sent to a commercial laboratory for taxonomic identification by Fatty Acid Methyl Ester (FAME) Analysis. FAME analysis revealed that three of the isolates had a high similarity index for biotypes of Pseudomonas flurorescens and two of the isolates had a high similarity index for biotypes of Pseudomonas putida. The identifications of the isolates are shown in Table 4. The five isolates identified in this manner were cultured, lyophilized, and submitted to the ARS Patent Culture Collection for deposit under the conditions of the Budapest Treaty on the International Recognition of the Deposit of Microorganisms for the Purpose of Patent Procedures on Jun. 13, 2001.

TABLE 4
Identification of Pseudomonas isolates releasing high GAF activity
into culture filtrates

| ISOLATE | IDENTIFICATION BY FAME ANALYSIS |
|---|---|
| WH6 | Pseudomonas fluroescens, Biotype C |
| E34 | Pseudomonas fluroescens, Biotype B |
| WH19 | Pseudomonas fluroescens, Biotype C |
| AH4 | Pseudomonas putida, Biotype B |
| AD31 | Pseudomonas putida, Biotype B |

The indicated Pseudomonas isolates were identified by Fatty Acid Methyl Ester (FAME) Analysis (Microcheck Inc., Microbial Analysis Lab (P.O. Box 456, Northfield, Vermont 05663).

### Example 3

### Biological Activity of the Germination-Arrest Factor (GAF) from Pseudomonas fluorescens isolates WH6 and E34

Summary

Bacteria-free culture filtrates of Pseudomonas fluorescens isolates WH6 and E34 irreversibly arrest the germination of seeds of annual bluegrass (Poa annua) at a stage immediately following emergence of the coleorhiza and plumule. Successive dilutions of the culture filtrates elicit a decreasing array of developmental disturbances, which are scored and used to estimate the relative concentration of GAF responsible for these effects. The effects of GAF are both species-specific and developmentally specific. Seeds of wheat, barley, and corn were unaffected by GAF treatment, but seed germination was arrested in a large number of other graminacious species, including a number of grassy weeds. The seeds of the dicots tested were unaffected.

In sensitive seeds, the effects of GAF are specific for the early stages of seed germination; foliar applications of GAF extracts or root immersion in GAF solutions had little if any effect on growth when these treatments were given at later stages in seedling development. In addition, the effects of GAF treatment on seed germination quickly become irreversible, with permanent arrest achieved in less than 24 hours. Furthermore, growth chamber tests of the efficacy of GAF in arresting the germination of seeds of annual bluegrass and jointed goatgrass sown on soils native to the Willamette Valley indicate that GAF is effective in soil systems as well as in the aqueous systems used in the standard bioassay for GAF activity.

A. Development of a Bioassay System for GAF Activity

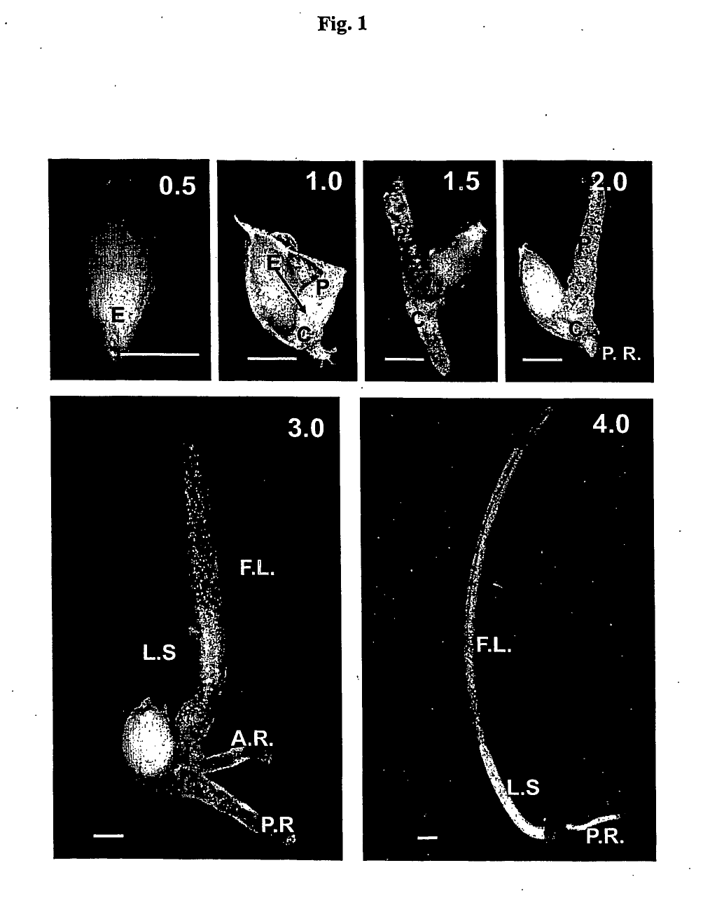

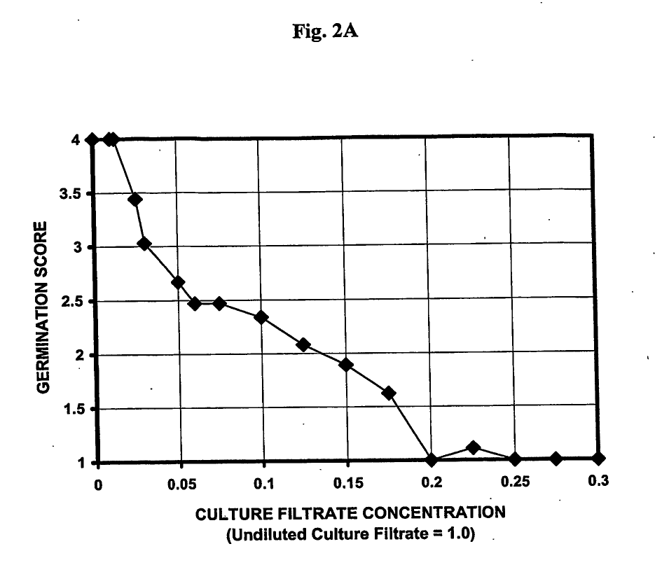

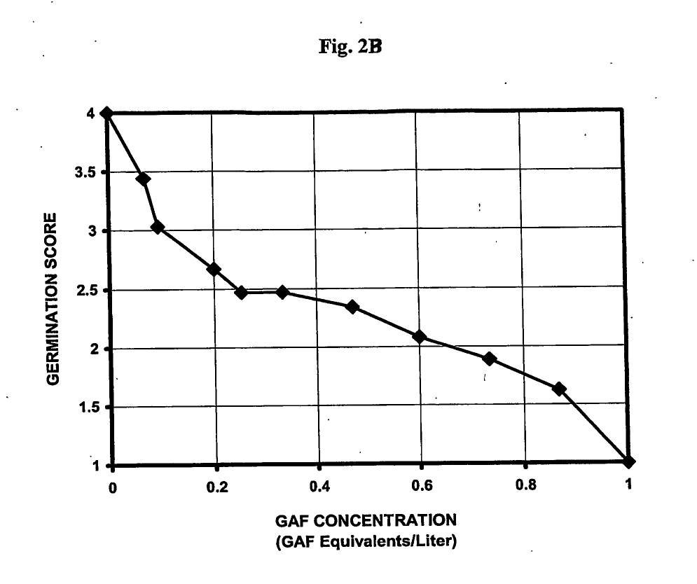

The effects of culture filtrates from Pseudomonas flurorescens isolates WH6 and E34 on the germination of seeds of annual bluegrass (Poa annua) were tested over a range of filtrate concentrations. When tested at full strength, these GAF-containing culture filtrates arrested germination of the annual bluegrass seeds at a stage immediately following emergence of the coleorhiza and plumule. Serial dilution of the culture filtrates resulted in a decreasing array of developmental disturbances. The developmental defects associated with varying GAF concentrations were characterized and used to develop a developmental scoring system for the estimation of GAF activity. This scoring system ranges from a Germination Score of zero (no visible germination of the seed occurs) to a Germination Score of four (seedling growth indistinguishable from that obtained with untreated seeds). A score of 0.5 is used for those cases where only the coleorhiza is visible, and score of one is reserved for cases of complete arrest immediately after emergence of the coleorhiza and plumule. The complete scoring system is summarized in Table 5. Digital images illustrating the various Germination Scores are shown in FIG. 1. The relationship between Germination Score and the concentration of GAF activity, tested in the Standard GAF Bioassay System (Example 1) using seeds of Poa annua, is illustrated by the graphs shown in FIGS. 2A and B.

B. Species-Specificity of GAF Effects on Seed Germination

The species-specificity of the respective GAF activities present in culture filtrates from Pseudomonas flurorescens isolates WH6 and E34 were evaluated in tests of the seeds from seventeen species of graminacious weeds and crop plants (Tables 6 and 7). The GAF activity from both isolates arrested the germination of seeds of Aegilops cylindrica (jointed goatgrass), Bromus tectorum (downy brome or cheatgrass), Vulpia myuros (rattail fescue), and six perennial and annual species of Poa. Seed germination was arrested in tall fescue and perennial ryegrass regardless of whether the grass cultivars used were infected with the fungal endophytes Neotyphodium coenophialum or N. loliae (e.g. cultivars Titan and Cutter) or free of these endophytes (e.g. cultivars A. U. Triumph and Linn). In contrast, germination of the seeds of spring wheat, barley, and maize was unaffected by GAF treatment. In addition, in bioassay tests conducted with WH6 filtrate, the germination of Avena fatua (wild oats) and Avena sativa (domestic oats) was arrested, but the germination of Oryza sativa (rice) was not affected (Table 7B).

Experiments were also conducted to determine the effects of GAF on the germination of the seeds of dicotyledonous plants (Tables 8A and 8B). Germination of surface-sterilized seeds of red clover, white clover, and Arabidopsis placed on agar mixed with live WH6 bacterial cultures was unaffected by the presence of the bacteria. Similarly, treatment of Arabidopsis with WH6 culture filtrate and GAF extracts prepared from this source in ten-fold concentrates resulted in somewhat reduced elongation of the roots and shoots of Arabidopsis seedlings, but no effect on seed germination was observed (FIG. 3). In addition, GAF from WH6 culture filtrate was tested on eight dicot species from seven plant families, with little or no effect on seed germination.

TABLE 5
Germination scoring system used to assess the effects of GAF activity on seed
germination and seedling development in Poa annua (annual bluegrass)

| GERMINATION | |
|---|---|
| SCORE | DEVELOPMENTAL CHARACTERISTICS |
| 0 | No visible seed germination. The seed has imbibed water, but the plumule and |
| | coleorhiza have not erupted through the seed coat. |
| 0.5 | Seed has imbibed water, and the embryo is white. The coleorhiza is just |
| | beginning to emerge from the seed, but the plumule has not yet emerged. |
| 1.0 | The plumule and coleorhiza have erupted through the seed coat. Coleorhizal |
| | hairs may be present, but no root emergence is observed. The plumule is |
| | chlorotic and greatly reduced in size relative to controls. |
| 1.5 | The plumule has expanded, but is not greater than the length of the seed, and is |
| | chlorotic. The primary root has elongated somewhat but is noticeably stunted. |
| 2.0 | The plumule is longer than the length of the seed, but is less than 1½ times the |
| | seed length, and is chlorotic. The primary root is elongated but stunted |
| | compared to controls. |
| 2.5 | The plumule is greater than 1½ times the seed length and remains chlorotic. |
| | The primary root is elongated but stunted compared to controls. |
| 3.0 | The first true leaf has emerged from the plumule and appears normal |
| | (with chlorophyll present) except that it is stunted in length when compared to |
| | controls. The primary root is elongated but also stunted. |
| 3.5 | The first true leaf is fully emerged and green in color. The primary root is |
| | elongated but stunted compared to controls. The first pair of adventitious |
| | lateral roots may be present, but they are also stunted compared to controls. |
| 4.0 | Normal germination. The first true leaf is fully emerged and green in color. |
| | The primary root is elongated, with the first pair of adventitious lateral roots |
| | beginning to emerge from the crown of the developing seedling. |

The developmental characteristics that define the various Germination Scores used to assess the effects of GAF on Poa annua seeds tested in the Standard GAF Bioassay System (see Example 1) are summarized above.

C. Developmental Specificity of GAF Effects on Seed Germination

The effects of GAF appear to be not only species-specific but developmentally specific, as well. The ability of GAF to specifically arrest the germination of sensitive seeds at a particular stage in the process is one of the biologically interesting features of this compound. Moreover, the effects of GAF appear largely limited to interfering with these very early stages of seedling development. Foliar sprays containing concentrated GAF extracts had little effect on the number of leaves, tillers, or plant dry weight of eight different graminacious species (Table 9). Immersion of the roots of these seedlings in GAF concentrates reduced overall plant dry weights in some species and had no effect on others (Table 10).

TABLE 6
The effects of culture filtrates from Pseudomonas fluorescens WH6
and E34 on the germination of various Poa species and cultivars

| | MEAN GERMINATION SCORE* | | |
|---|---|---|---|
| | (±Standard Error of the Mean) | | |
| | For the Indicated Treatment | | |
| POA SPECIES | | E34 | WH6 |
| AND CULTIVARS | H₂O | Culture Filtrate | Culture Filtrate |
| P. alpina cv Alpine | 3.9 ± 0.1 | 1.8 ± 0.1 | 1.6 ± 0.1 |
| P. annua (Wild-Type) | 4.0 ± 0.0 | 1.0 ± 0.0 | 1.0 ± 0.0 |
| P. glauca cv Tundra | 3.0 ± 0.1 | 1.0 ± 0.0 | 1.0 ± 0.0 |
| P. glauca cv Unknown | 3.3 ± 0.1 | 1.7 ± 0.1 | 1.5 ± 0.1 |
| P. pratensis cv | 3.7 ± 0.1 | 1.0 ± 0.0 | 1.0 ± 0.0 |
| Viva Kentucky | | | |
| P. pratensis cv | 3.6 ± 0.2 | 1.0 ± 0.0 | 1.0 ± 0.0 |
| Gnome Kentucky | | | |
| P. supine cv Supra Nova | 3.6 ± 0.1 | 2.0 ± 0.1 | 2.3 ± 0.1 |
| P. trivialis cv | 2.8 ± 0.1 | 1.0 ± 0.0 | 1.0 ± 0.0 |
| Cypress Roughstalk | | | |
| P. trivialis (Wild-Type) | 3.0 ± 0.1 | 1.0 ± 0.0 | 1.0 ± 0.0 |
| Seeds of the indicated species and cultivars were tested for their response to the GAF activity present in culture filtrates from WH6 and E34. Bioassays were performed in 48 well tissue culture clusters. Surface sterilized seeds were imbibed in sterile distilled water for 48 hr (15° C., 8 hr day at 50 μmol/m²/s; | | | |
| # 10° C., 16 hr dark period), and individual seeds were transferred to a well which contained 1.0 ml culture filtrate from WH6 or E34. The plates were sealed and placed at 20° C. 50 μmol/m²/s for 48 hr. Treatment liquids were then removed and replaced with sterile distilled water (150 μl per | | | |
| # well) to facilitate germination. Three seeds were used per treatment with three replicates per trial and the experiment was repeated. Seeds exposed to sterile distilled water were used as non-treated control. Germination was scored as in the Standard GAF Bioassay System (see Table 5). | | | |

*Germination Score of 1.0 = Germination completely arrested immediately after emergence of the coleorhiza and plumule. Germination Score of 4.0 = Normal germination and seedling development. (See Table II-1 for complete details of the scoring system.)

D. Irreversible Nature of GAF Effects on Seed Germination

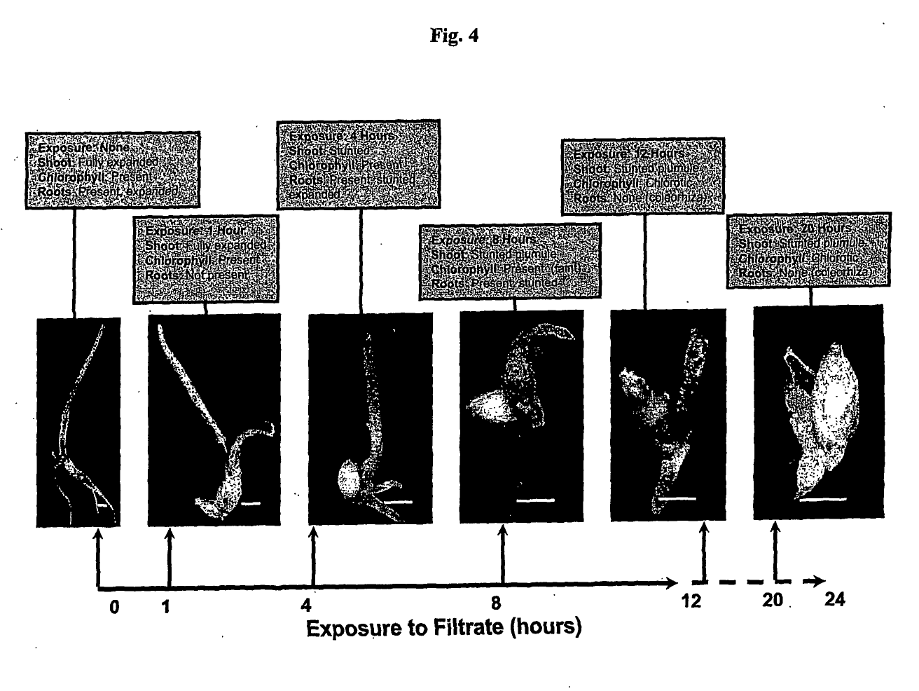

The time course of GAF effects on seed germination was investigated using seeds of Poa annua. Seeds were allowed to imbibe water for 48 hours prior to exposure to varying concentrations of WH6 culture filtrate for periods of one to 24 hours. Following the exposure to culture filtrate for varying time periods, the seeds were thoroughly rinsed with distilled water to remove GAF activity and then placed in a growth chamber for seven days under conditions favorable for seed germination. With even one hour of exposure to WH6 culture filtrate, primary root development was severely inhibited, and after 20 hours of exposure to the filtrate, germination was completely and irreversibly inhibited (FIG. 4).

TABLE 7A
The effects of culture filtrates from Pseudomonas fluorescens WH6
and E34 on the germination of several important graminaceous
weeds and crop species

| | MEAN GERMINATION SCORE* | | |
|---|---|---|---|
| | (±Standard Error of the Mean) | | |
| | For the Indicated Treatment | | |
| SPECIES | | E34 | WH6 |
| AND CULTIVARS | H₂O | Culture Filtrate | Culture Filtrate |
| Aegilops cylindrica | 3.9 ± 0.1 | 1.2 ± 0.1 | 1.5 ± 0.2 |
| (Wild-Type) | | | |
| Bromus tectorum | 3.1 ± 0.2 | 1.1 ± 0.1 | 1.2 ± 0.2 |
| (Wild-Type) | | | |
| Festuca arundenceae cv | 3.4 ± 0.2 | 1.3 ± 0.1 | 1.4 ± 0.1 |
| A. U. Triumph | | | |
| Festuca arundenceae | 3.9 ± 0.1 | 1.0 ± 0.0 | 1.0 ± 0.0 |
| cv Titan | | | |
| Hordeum vulgare cv Morex | 2.3 ± 0.2 | 2.1 ± 0.4 | 2.0 ± 0.3 |
| Lolium perenne cv Cutter | 3.6 ± 0.1 | 1.1 ± 0.1 | 1.4 ± 0.1 |
| Lolium perenne cv Linn | 3.2 ± 0.2 | 1.0 ± 0.0 | 1.1 ± 0.1 |
| Triticum sativum cv | 3.5 ± 0.2 | 3.0 ± 0.0 | 3.0 ± 0.0 |
| Madsen | | | |
| Vulpia myuros (Wild-Type) | 4.0 ± 0.0 | 1.0 ± 0.0 | 1.0 ± 0.0 |
| Zea mays cv W22 | 3.9 ± 0.1 | 3.6 ± 0.2 | 3.9 ± 0.1 |
| (blue corn) | | | |
| Zea mays cv | 4.0 ± 0.0 | 4.0 ± 0.0 | 4.0 ± 0.0 |
| B73 (yellow corn) | | | |
| Seeds of the indicated species were tested for their response to the GAF activity present in culture filtrates from WH6 and E34. Bioassays of small seeded species were performed in 48 well tissue culture clusters, while bioassays of large seeded species were performed in 24 well tissue culture clusters. Surface sterilized seeds were imbibed in sterile distilled water for 48 hr (15° C., 8 hr day at | | | |
| # 50 μmol/m²/s; 10° C., 16 hr dark period), and individual seeds were transferred to a well that contained 1.0 (48-well clusters) or 2.0 (24-well clusters) ml culture filtrate from WH6 or E34. The plates were sealed and placed at 20° C. 50 μmol/m²/s for 48 hr. Treatment liquids were then removed and replaced | | | |
| # with sterile distilled water (150 or 400 μl/well in 48- and 24-well plates, respectively) to facilitate germination. Three seeds were used per treatment with three replicates per trial and the experiment was repeated. Seeds exposed to sterile distilled water were used as non-treated control. Germination | | | |
| # was scored as in the Standard GAF Bioassay System (see Table 5). | | | |

*Germination Score of 1.0 = Germination completely arrested immediately after emergence of the coleorhiza and plumule. Germination Score of 4.0 = Normal germination and seedling development. (See Table 5 for complete details of the scoring system.)

E. Efficacy of WH6 GAF Extracts in Arresting Seed Germination in Soil Systems

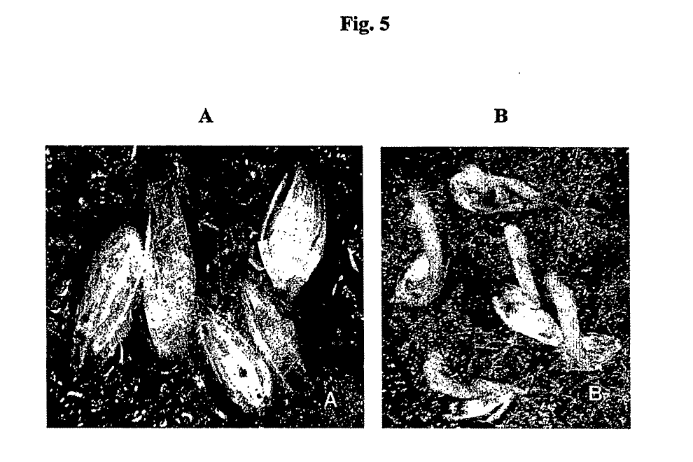

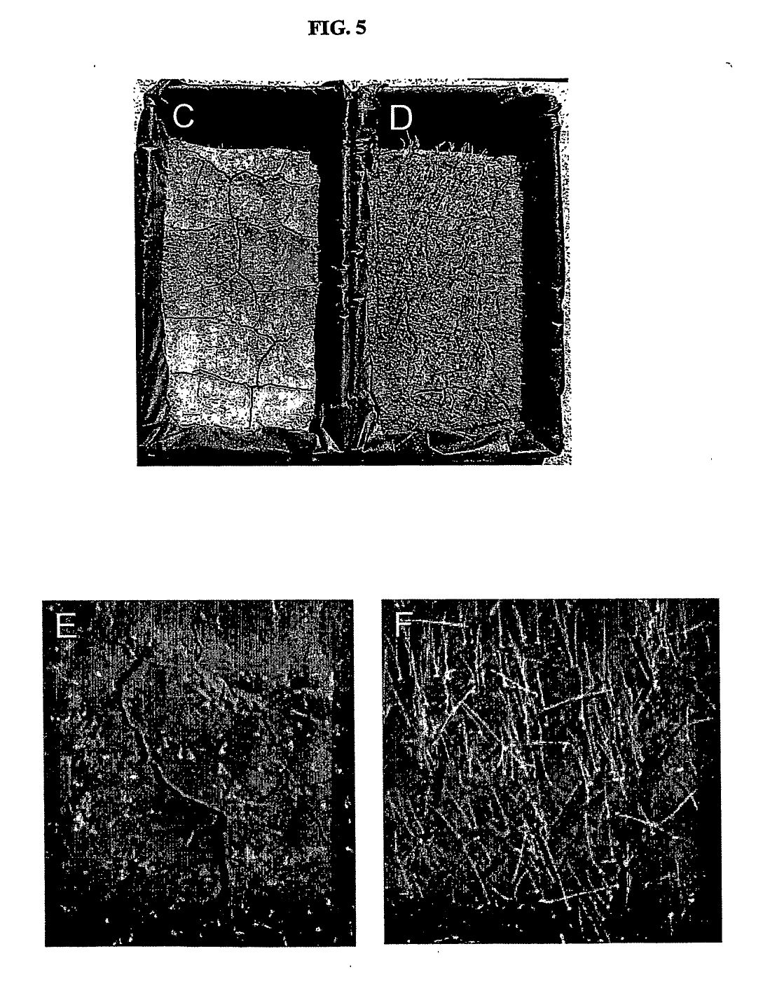

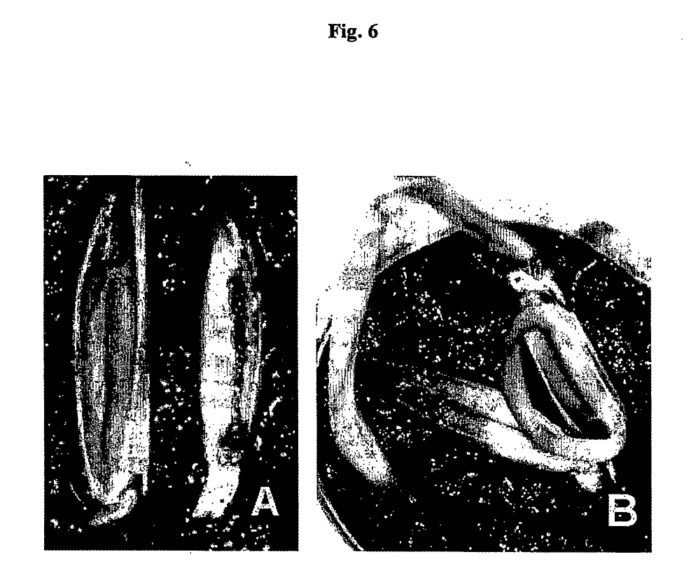

The ability of GAF extracts (prepared from WH6 culture filtrates) to arrest the germination of seeds of annual bluegrass and jointed goatgrass was tested and compared in the standard (aqueous) bioassay system and in growth chamber tests using seeds germinated on native soils. As shown in FIGS. 5 and 6 and Tables 11 and 12, GAF extracts completely inhibited or arrested germination of the seeds of annual bluegrass and jointed goat grass in both the standard bioassay system and in tests using soil systems.

TABLE 7B
The effects of culture filtrate from Pseudomonas fluorescens WH6
on the germination of Avena fatua (wild oats), Avena sativa (oats)
and Oryza sativa (rice)

MEAN GERMINATION
SCORE* (±Standard
Error of the Mean)
For the Indicated Treatment

| | | WH6 |
|---|---|---|
| SPECIES AND CULTIVARS | H₂O | Culture Filtrate |
| Avena sativa cv Cayuse | 4.0 ± 0.0 | 1.25 ± 0.04 |
| Avena fatua (Wild type) | 4.0 ± 0.0 | 1.90 ± 0.2 |
| Oryza sativa cv Cypress | 4.0 ± 0.0 | 3.5 ± 0.0 |
| Seeds of the indicated species were tested for their response to the GAF activity present in culture filtrate from WH6. Bioassays were performed in sterile glass petri dishes containing blue blotter paper saturated with either GAF or H₂O. The seed “hull” from seeds of Avena fatua were removed to | | |
| # facilitate germination prior to surface sterilization. Ten surface sterilized seeds were aseptically transferred to the surface of each blotter paper and the lids sealed. The dishes were placed into a germination chamber (25° C., 8 hr day at 50 μmol/m²/s, 16 hr dark period), and the seeds allowed to | | |
| # germinate. There were 10 seeds per treatment and the experiment was repeated. Seeds exposed to sterile distilled water were used as non-treated control. Germination was scored as in the Standard GAF Bioassay System (see Table 5). | | |

*Germination Score of 1.0 = Germination completely arrested immediately after emergence of the coleorhiza and plumule. Germination Score of 4.0 = Normal germination and seedling development. (See Table 5 for complete details of the scoring system.)

TABLE 8A
The effects of live cultures of Pseudomonas fluorescens WH6
on the germination of some dicotyledonous plants

| | MEAN GERMINATION SCORE* |
|---|---|
| PLANT SPECIES | (±Standard Error of the Mean) |
| Trifolium repens (white clover) | |
| Untreated Controls | 4.0 ± 0.0 |
| Treated Seeds | 4.0 ± 0.0 |
| Trifolium pratense (red clover) | |
| Untreated Controls | 4.0 ± 0.0 |
| Treated Seeds | 4.0 ± 0.0 |
| Arabidopsis thaliana | |
| Untreated Controls | 4.0 ± 0.0 |
| Treated Seeds | 3.5 ± 0.0 |

Surface-sterilized seeds of the indicated species of dicotyledonous plants were sown onto 0.9% agar mixed with live WH6 bacterial cultures (10% v/v). The agar cultures were placed in a growth chamber under conditions favorable to seed germination. Germination Scores, based on the scoring system described in Table 5, were determined after 7-days.
*Germination Score of 1.0 = Germination completely arrested immediately after emergence of the coleorhiza and plumule. Germination Score of 4.0 = Normal germination and seedling development. (See Table 5 for complete details of the scoring system.)

TABLE 8B
The effects of culture filtrate from Pseudomonas fluorescens WH6 on the germination
of the seeds of selected dicotyledonous plants

| | | | | Control | | | |
|---|---|---|---|---|---|---|---|
| | | Control | | PMS w/o | | WH6 | |
| | | H₂O | | glucose | | Culture Filtrate | |
| SPECIES AND COMMON NAMES | Plant Family | (% Germ.) | | (% Germ.) | | (% Germ.) | |
| Chrysanthemum maximum Shasta daisy | Asteraceae | 10/12 | (83) | 6/12 | (50) | 12/12 | (100) |
| Menthua piperita Peppermint | Lamiaceae | 7/9 | (78) | 5/9 | (56) | 6/9 | (67) |
| Antirrhinum majus Snapdragon | Scrophulariaceae | 9/9 | (100) | 5/9 | (56) | 7/9 | (78) |
| Petunia hybrida Hybrid Petunia | Solanaceae | 8/9 | (89) | 6/9 | (67) | 7/9 | (78) |
| Raphanus sativus Radish | Brassicaceae | 12/12 | (100) | 4/12 | (33) | 7/12 | (58) |
| Lactuca sativa Leaf lettuce | Asteraceae | 12/12 | (100) | 12/12 | (100) | 12/12 | (100) |
| Daucus carota Carrot | Aplaceae | 12/12 | (100) | 11/12 | (92) | 10/12 | (83) |
| Lycopersicon esculentum Tomato | Solanaceae | 12/12 | (100) | 10/12 | (83) | 8/12 | (67) |
| Seeds of the indicated species were tested for their response to the GAF activity present in culture filtrate from WH6. Bioassays were performed in 48 well tissue culture clusters placed into a germination chamber (20° C. 50 μmol/m²/s). Surface sterilized seeds were placed into 1.0 ml of filtrate from WH6 or PMS medium without | | | | | | | |
| # glucose (control for culture medium) or H₂O (positive germination control). Percent germination was determined for each species when the H₂O treated seeds reached 100% germination or at 10 days. | | | | | | | |

TABLE 9
Effects of foliar applications of GAF concentrates derived from
P. fluorescens WH6 on the growth of seedlings of
graminacious plant species

| | | | Number of leaves and | |
|---|---|---|---|---|
| | Dry weight (grams) at | | tillers at | |
| Species and | 5-weeks (±SEM) | | 4-weeks (±SEM) | |
| Cultivars | Shoots | Roots | Leaves | Tillers |
| Aegilops cylindrica | | | | |
| (Wild-Type) | | | | |
| Buffer Control | 0.72 ± 0.04 | 0.88 ± 0.08 | 21.6 ± 1.7 | 7.1 ± 0.4 |
| GAF Treatment | 0.73 ± 0.04 | 0.83 ± 0.07 | 19.9 ± 1.4 | 7.0 ± 0.4 |
| (10X)* | | | | |
| Bromus tectorum | | | | |
| (Wild-Type) | | | | |
| Buffer Control | 0.72 ± 0.02 | 0.55 ± 0.05 | 49.5 ± 4.1 | 14.2 ± 1.1 |
| GAF Treatment | 0.63 ± 0.02 | 0.58 ± 0.05 | 50.6 ± 1.9 | 15.2 ± 0.8 |
| (10X)* | | | | |
| Festuca | | | | |
| arundenceae | | | | |
| cv Shenandoah | | | | |
| Buffer Control | 0.29 ± 0.04 | 0.13 ± 0.02 | 17.6 ± 1.8 | 5.7 ± 0.7 |
| GAF Treatment | 0.30 ± 0.05 | 0.14 ± 0.02 | 16.2 ± 1.8 | 5.5 ± 0.5 |
| (10X)* | | | | |
| Lolium perenne | | | | |
| cv Morning Star | | | | |
| Buffer Control | 0.32 ± 0.03 | 0.22 ± 0.03 | 40.1 ± 2.8 | 11.5 ± 0.7 |
| GAF Treatment | 0.39 ± 0.03 | 0.29 ± 0.03 | 45.0 ± 2.7 | 12.0 ± 0.6 |
| (10X)* | | | | |
| Poa annua | | | | |
| Buffer Control | 0.55 ± 0.08 | 0.43 ± 0.05 | 43.3 ± 5.6 | 12.9 ± 1.6 |
| GAF Treatment | 0.55 ± 0.05 | 0.41 ± 0.05 | 45.2 ± 3.2 | 12.6 ± 0.9 |
| (10X)* | | | | |
| Triticum sativum | | | | |
| cv Madsen | | | | |
| Buffer Control | 0.84 ± 0.03 | 0.56 ± 0.03 | 13.1 ± 0.8 | 3.5 ± 0.2 |
| GAF Treatment | 0.80 ± 0.02 | 0.51 ± 0.02 | 14.0 ± 0.6 | 3.5 ± 0.2 |
| (10X)* | | | | |
| Triticum sativum | | | | |
| cv Penewawa | | | | |
| Buffer Control | 0.97 ± 0.04 | 0.39 ± 0.02 | 14.3 ± 0.3 | 3.6 ± 0.2 |
| GAF Treatment | 0.90 ± 0.04 | 0.40 ± 0.03 | 15.0 ± 0.6 | 3.5 ± 0.2 |
| (10X)* | | | | |
| Vulpia myuros | | | | |
| (Wild-Type) | | | | |
| Buffer Control | 0.45 ± 0.04 | 0.33 ± 0.02 | 76.7 ± 3.3 | 23.4 ± 1.0 |
| GAF Treatment | 0.44 ± 0.04 | 0.34 ± 0.03 | 77.2 ± 5.5 | 24.5 ± 1.6 |
| (10X)* | | | | |
| Seedlings of the indicated graminacious species were allowed to develop to the two-leaf stage and then transplanted into vermiculite. The leaves of the seedlings were then sprayed to saturation with a concentrated GAF solution or a buffer control solution. To prepare the concentrated GAF solution, culture filtrates derived from Pseudomonas fluorescens WH6 were taken to dryness in vacuo at 45° C. | | | | |
| # and then extracted three times with 90% (v/v) ethanol using ethanol volumes equivalent to one-third of the original culture filtrate volume for each extraction. The combined extracts were taken to dryness in vacuo at 45° C. and redissolved in a volume of 0.05 M KH₂PO₄ buffer (pH 6.5, KOH) equal to one-tenth of the original culture filtrate volume. Following the foliar application of the GAF | | | | |
| # concentrate and the control solution, the seedlings were grown in the greenhouse with weekly applications of a 20:20:20 N:P:K fertilizer. The number of leaves and tillers were determined at weekly intervals for 4 weeks. The seedlings were harvested and shoot and root dry weights determined at 5 weeks post-treatment. | | | | |

*A GAF concentration of 10X is equivalent to dissolving the GAF extract in a volume equal to one-tenth of the original culture filtrate volume from which it was derived.

TABLE 10
Effects of transient immersion of roots in GAF concentrates on
the growth of seedlings of graminacious plant species

| | DRY WEIGHT AT 5-WEEKS | |
|---|---|---|
| | (Grams ± Standard Error of the Mean) | |
| SPECIES AND CULTIVARS | Shoots | Roots |
| Aegilops cylindrica (Wild-Type) | | |
| Buffer Control | 0.28 ± 0.04 | 0.17 ± 0.03 |
| GAF Treatment (10X)* | 0.21 ± 0.04 | 0.14 ± 0.03 |
| Bromus tectorum (Wild-Type) | | |
| Buffer Control | 0.11 ± 0.02 | 0.08 ± 0.02 |
| GAF Treatment (10X)* | 0.08 ± 0.02 | 0.07 ± 0.01 |
| Lolium perenne cv Morning Star | | |
| Buffer Control | 0.10 ± 0.01 | 0.06 ± 0.01 |
| GAF Treatment (10X)* | 0.05 ± 0.01 | 0.03 ± 0.01 |
| Triticum sativum cv Madsen | | |
| Buffer Control | 0.46 ± 0.03 | 0.25 ± 0.02 |
| GAF Treatment (10X)* | 0.30 ± 0.03 | 0.20 ± 0.04 |
| Triticum sativum cv Penewewa | | |
| Buffer Control | 0.56 ± 0.04 | 0.19 ± 0.01 |
| GAF Treatment (10X)* | 0.35 ± 0.02 | 0.13 ± 0.01 |
| Vulpia myuros (Wild-Type) | | |
| Buffer Control | 0.07 ± 0.01 | 0.04 ± 0.01 |
| GAF Treatment (10X)* | 0.05 ± 0.01 | 0.02 ± 0.00 |
| Seedlings of the indicated graminacious species were allowed to develop to the two-leaf stage. The roots of these seedlings were then immersed for 18 hours in either a concentrated GAF solution (derived from Pseudomonas fluorescens WH6) or in the corresponding buffer control. (The 10X GAF concentrate and buffer control were prepared as described in the legend to Table 9.) Following GAF treatment, the roots of the seedlings were washed thoroughly to remove any traces of the GAF | | |
| # solution. The seedlings were then planted into vermiculite and fertilized weekly with 20:20:20 N:P:K fertilizer. The seedlings were harvested, and shoot and root dry weights were determined five weeks after the GAF treatment. | | |

*A GAF concentration of 10X is equivalent to dissolving the GAF extract in a volume equal to one-tenth the original culture filtrate volume from which it was derived.

TABLE 11
Efficacy of GAF extracts derived from Pseudomonas fluorescens
WH6 in arresting germination of the seeds of Poa annua
(annual bluegrass) in soil systems

| | MEAN GERMINATION SCORE* | | |
|---|---|---|---|
| | (±Standard Error of the Mean) | | |
| | AT THE RELATIVE GAF | | |
| | CONCENTRATION (X) INDICATED** | | |
| IMBIBING SOLUTION | 1X | 5X | 15.0X |
| WATER | 4.0 ± 0.0 | 0.5 ± 0.2 | 0.0 ± 0.0 |
| GAF CONCENTRATE | 4.0 ± 0.0 | 0.0 ± 0.0 | 0.0 ± 0.0 |
| Seeds of Poa annua were placed on dry soil contained in the outer wells of a Netwell tissue culture cluster. In each outer well, five seeds of Poa annua were placed onto 1.5 grams of dry Woodburn sandy-loam soil. A total of four wells were prepared for each seed treatment. One ml of distilled water or one ml of the appropriate GAF concentrate was added to each well, and the seeds were | | | |
| # allowed to imbibe in the dark for 48 hours at 15° C. At the end of the imbibition period, one ml of the indicated GAF solution was added to each well, and the seeds were placed in a growth chamber at 15° C. with an 8-hour photoperiod. Seed germination was evaluated at 7 and 12 days post treatment. GAF extracts and concentrates were prepared from culture filtrates of Pseudomonas fluorescens WH6 | | | |
| # as described in the legend to Table 9 by dissolving 90% ethanol extracts of dried culture filtrates in a volume of 0.05 M KH₂PO₄ buffer (pH 6.5, KOH) equal to 1/15 of the original culture filtrate volume and diluting as indicated. | | | |

*Germination Score of 0.0 = Germination completely inhibited. Germination Score of 4.0 = Normal germination and seedling development. (See Table 5 for complete details of the scoring system.)
**A GAF concentration of 15X is equivalent to dissolving the GAF extract in a volume equal to one-fifteenth of the original culture filtrate volume from which it was derived.

TABLE 12
Efficacy of GAF extracts derived from Pseudomonas fluorescens
WH6 in arresting germination of the seeds of Aegilops cylindrica
(jointed goatgrass) in soil systems

| | MEAN GERMINATION SCORE* | | |
|---|---|---|---|
| | (±Standard Error of the Mean) | | |
| | AT THE RELATIVE GAF | | |
| | CONCENTRATION (X) INDICATED** | | |
| IMBIBING SOLUTION | 1X | 5X | 15.0X |
| WATER | 4.0 ± 0.0 | 0.8 ± 0.2 | 0.6 ± 0.1 |
| GAF CONCENTRATE | 2.7 ± 0.3 | 0.8 ± 0.2 | 0.5 ± 0.0 |
| Seeds of Aegilops cylindrica were placed on dry soil contained in the outer wells of a Netwell tissue culture cluster. In each outer well, five seeds of Aegilops cylindrica were placed onto 1.5 grams of dry Woodburn sandy-loam soil. A total of 4 wells were prepared for each seed treatment. One ml of distilled water was added to each well, and the seeds were allowed to imbibe in the dark for 48 hours | | | |
| # at 15° C. At the end of the imbibition period, one ml of the appropriate indicated GAF solution (or control solution) was added to each well, and the seeds were placed in a growth chamber at 15° C. with an 8-hour photoperiod. Seed germination was evaluated at 7 and 12 days post treatment. (GAF extracts and concentrates were prepared from culture filtrates of Pseudomonas fluorescens WH6 as described in the legend to Table II-4.) | | | |

*Germination Score of 0.0 = Germination completely inhibited. Germination Score of 4.0 = Normal germination and seedling development. (See Table II-1 for complete details of the scoring system.)
**A GAF concentration of 15X is equivalent to dissolving the GAF extract in a volume equal to one-fifteenth of the original culture filtrate volume from which it was derived.

### Example 4

### Genes Controlling the Expression of Germination-Arrest Factor (GAF) Activity in Culture Filtrates of Pseudomonas fluorescens WH6

Summary

The discovery of genes that control either production or secretion of the Pseudomonas Germination Arresting Factor (GAF) was accomplished by transposon mutagenesis. Transposon Tn5, which confers tetracycline resistance, was introduced into P. fluorescens wild-type strain WH6 on plasmid pUTmini-Tn5gfp (Tn5gfp). Of 1,214 tetracycline-resistant transformants screened, three were determined to have no GAF activity in culture filtrates (Table 13). DNA fragments containing Tn5 were cloned from each of the three mutants and used as molecular probes to identify wild-type DNA fragments by sequence homology. These wild-type fragments were cloned into pME4510 and used in complementation studies to identify genes that would restore GAF activity in the respective Tn5 mutants. The three Tn5 mutants, designated gaf1, gaf2 and gaf3, were complemented with wild-type DNA fragments. The nucleotide sequences of the wild-type genes, GAF2 and GAF3, have varying degrees of homology with known genes (a phosphonbosylglycinamide formyltransferase enzyme from Rhodococcus fascians and the dicistronic operon prtIR from P. fluorescens strain LS107d2, respectively). The gaf1 mutation was complemented by a DNA fragment that is predicted to encode a putative 459 amino acid polypeptide that, without being bound by theory, is believed to be essential either for expression or secretion of GAF activity. This putative polypeptide has amino acid sequence identity with a predicted polypeptide identified from the Pseudomonas fluorescence NCBI Microbial Genomes Annotation Project (GI:2306727). No function was attributed to this predicted protein. The complemented mutants exhibited restored GAF activity, establishing that the DNA sequences in question actually code for products essential for the expression of GAF activity in the bacterial culture filtrate.

Without being bound by theory, the GAF nucleotide and amino acid sequences identified herein, and functional fragments thereof, may encode GAF peptides, or may encode peptides involved in the production or secretion of GAF.

A. Characterization of the GAF1 Mutation of Pseudomonas fluorescens Wh6-1

A Tn5-containing Pst-HindIII fragment was cloned from the WH6-1 mutant. SEQ ID NO: 1 is the Pseudomonas nucleotide sequence flanking one side of Tn5. Polymerase chain reaction (PCR) was used to amplify a portion of the Pseudomonas sequences, and a probe was constructed of the PCR product using a non-radioactive kit (Amersham Pharmacia Biotech). A probe made to those sequences was used to identify a 5.2 kb EcoRI fragment from WH6 by sequence homology (SEQ ID NO: 2). This fragment was cloned into pBlueScript (pOSU5201) for nucleotide sequencing, and into pME4510 (Rist and Kertesz, FEMS Microbiology Letters 169:179-83, 1998), a shuttle vector that replicates in Escherichia coli and Pseudomonas spp., for genetic complementation studies. A BLAST search for sequence identity with entries in the NCBI database identified no entries with stretches of homology. This fragment is 5,178 nucleotides in length and the site of Tn5 insertion was within two open reading frames, one on each strand, that could encode putative polypeptides of 459 (ORFc, SEQ ID NO: 3) and 351 (ORFe, SEQ ID NO: 4) amino acids, respectively. The gaf1 mutation was initially complemented by this 5.2 kb fragment, resulting in the restoration of GAF activity (Table 14).

ORFe (SEQ ID NO: 4) has a methionine translation initiation codon but a search of the ExPASy (Expert Protein Analysis System; proteomics server of the Swiss Institute of Bioinformatics) and NCBI databases revealed no polypeptides with sequence identity. While not being bound by theory, ORFc (SEQ ID NO: 3) is believed to be essential for expression or secretion of GAF activity. This putative polypeptide has amino acid sequence identity with a predicted polypeptide identified from the Pseudomonas fluorescence NCBI Microbial Genomes Annotation Project (GI:2306727). ORFc (SEQ ID NO: 3) has 363/457 (79%) identities and 391/457 (85%) positive amino acids with the predicted polypeptide from P. fluorescens, and the nucleotide sequence of this fragment corresponds with gene Pflu_p_{—}2874 identified from the Pseudomonas fluorescence NCBI Microbial Genomes Annotation Project No function was attributed to this predicted protein. ORFc has two conserved domains; one spanning amino acids 55-170 is characteristic of a serine/threonine protein kinase domain. The second includes amino acids 283 through 446 and is characteristic of a RIO-like kinase domain. While not being bound by theory, this suggests that this protein could be involved in phosphorylating a specific protein that is also essential for GAF activity.

The translation start of ORFc lies approximately 60 nucleotides downstream of the PstI cloning site of pOSU2400. POSU2400 has been recovered from genetically complemented cells of WH6-1, suggesting that the promoter for this gene either resides within these 60 nucleotides, or that transcription is initiated from a promoter within vector sequences on pOSU2400. If the promoter is not in the insert of pOSU2400, the 5.2 kb insert in pOSU5200 contains 2,800 nucleotides upstream of the PstI cloning site that would contain the promoter.

A terminal 2373 bp EcoRI-PstI fragment also contains the site of Tn5 insertion (SEQ ID NO: 5). When a construct with this fragment (pOSU2400) was used to transform WH6-1, GAF activity was restored in the bacterial culture filtrate, indicating that all of the genetic information lost to Tn5 insertion resides within this smaller fragment.

B. Characterization of the gaf2 Mutation of Pseudomonas fluorescens Strain Wh6-2

A Tn5-containing PstI-HindIII fragment was cloned from the WH6-2 mutant SEQ ID NO: 6 is the Pseudomonas nucleotide sequence flanking one side of Tn5. A probe made to those sequences was used to identify a 5 kb BamHI fragment from WH6 by sequence homology (SEQ ID NO: 7). This fragment was cloned into pME4510 (pOSU5000) and pBlueScript (pOSU5501) for sequencing. The insert in pOSU5000 complements the gaf1 mutation, resulting in restoration of GAF activity (Table 14). The insert inpOSU5001 was 5040 nucleotides in length. A BLAST search for sequence identity with entries in the NCBI database identified a putative gene from Rhodococcus facians that had partial sequence identity with GAF2, (Accession Number: AJ311775.1; GI Number: GI:14595063). The gene encodes a phosphonbosylglycinamide formyltransferase.

The BamHI fragment contains an open reading frame (ORFb, SEQ ID NO: 8) that spans the site of Tn5 insertion and could encode a putative polypeptide of 200 amino acids. A search of bacterial proteins with ExPASy revealed a region of highest similarity with a phosphoribosylglycinamide formyltransferase enzyme from Rhodococcus fascians; the alignment of 164 amino acids revealed 37 percent (61/164) to be identical and 52 percent similar with a single gap.

Methionyl-tRNA formyltransferase transfers a formyl group onto the amino terminus of the acyl moiety of the methionyl aminoacyl-tRNA. The formyl group appears to play a dual role in the initiator identity of N-formylmethionyl-tRNA by promoting its recognition by IF2 and by impairing its binding to EFRU-GTP. Formyltetrshydrofolate dehydrogenase produces formate from formyl-tetrahydrofolate. This is the N-terminal domain of these enzymes and is found upstream of the C-terminal domain. The trifunctional glycinanude ribonucleotide synthetase-aminoimidazole ribonucleotide synthetase-glycinamide ribonucleotide transformylase catalyses the second, third and fifth steps in de novo purine biosynthesis. The glycinamide ribonucleotide transformylase belongs to this group.

Without being bound by theory, it appears that the gaf2 mutation may affect a putative formyltransferase gene that is essential for the synthesis, activation or secretion of GAF. This formyltransferase has not been described for P. fluorescens.

C. Characterization of the gaf3 Mutation of Pseudomonas fluorescens Strain Wh6-3

The gaf3 mutation of Pseudomonas fluorescens WH6-3 exhibits a slower growth rate than the parent strain WH6, and culture filtrates are devoid of any detectable GAF activity when measured in the Standard GAF Bioassay System as described in Example 1 (Table 13).

A PstI-HindIII fragment was cloned from strain WH6-3 that contains a portion of the Tn5 element that encodes tetracycline resistance at the HindIII end, and flanking Pseudomonas sequences near the PstI end. SEQ ID NO: 9 is the Pseudomonas nucleotide sequences at the PstI end. PCR was used to amplify a portion of the Pseudomonas sequences, and a probe was constructed of the PCR product using a non-radioactive kit (Amersham Pharmacia Biotech). A PstI fragment approximately 3.4 kb is size was identified in strain WH6 by Southern blot hybridization (SEQ ID NO: 10) to have homology with the probe and this fragment was subsequently cloned into pME4510. The insert in this construct (pOSU3400) was determined to be 3,279 bp in length The gaf3 mutant was transformed with this construct and GAF activity was restored to culture filtrates (Table 14) as determined in the Standard GAF Bioassay System.

The site of Tn5 insertion was in DNA that is highly homologous to the dicistronic operon, prtIR, which was previously cloned from P. fluorescens strain LS 107d2 (Burger et al., Microbiology 146:3149-55, 2000). This operon consists of two genesprtlandprtR and Tn5 was determined to have inserted within prtR in strain WH6-3. The first gene, prtI, is presumed to encode a sigma factor that is involved with extracytoplasmic functions (ECF sigma factors of the σ⁷⁰ family). The second gene, prtR, may be related to either a group of membrane-associated anti-sigma factors or some transmembrane-associated regulators. Evidence suggests that PrtR in LS107d2 is a transmembrane-associated regulator (Burger et al., Microbiology 146:3149-55, 2000). SEQ ID NO: 11 is the amino acid sequence for the putative PrtI polypeptide encoded by strain WH6. Comparison of amino acid sequences encoded by prtI from strain WH6 and LS107d2 revealed nearly complete identity to the PstI site used in cloning the PstI fragment from strain WH6. The PstI site (CTGCAG) is not present in strain LS107d2 (CTGCAA), indicating that either a transition had occurred from A to G (last nucleotide) in strain WH6, or a transition occurred from G to A in LS107d2 at this site. The G present at this site generates a PstI site (CTGCAG) in WH6. The open reading frame of prtI in strain LS107d2 continues upstream of that site resulting in an additional 17 amino acids that are not encoded by the PstI fragment from WH6. It is predicted that the DNA sequences upstream of the PstI site of the Prt1 gene of WH6 will be homologous to corresponding DNA in the Prt1 gene of strain LS107d2. The sequence of the PrtIR operon and upstream, flanking noncoding DNA of strain LS107d2 can be found using Accession Number AF228767; GI Number GI:8895712 at the NCBI site. Because the PstI fragment cloned from WH6 provides genetic complementation of gaf3, it is likely that transcription originates from vector sequences enabling expression of the downstream intact prtR gene.

SEQ ID NO: 12 is the amino acid sequence for the putative PrtR polypeptide encoded by strain WH6. Comparison of amino acid sequences encoded by prtR from strain WH6 and LS107d2 revealed that the polypeptides are 84 percent identical and have 88 percent positive amino acids. Because there may be multiple sigma factors, it is unclear whether the gene cloned from WH6 is one of a gene family. The ECF sigma factors are necessary for transcription of genes that encode products with extracytoplasmic functions. Mutation of either the prtIR operon described by Burger et al. (Microbiology 146:3149-55, 2000), or similar ECF sigma factor in strain WH6-3, appears to interfere with expression of the gene(s) that encode GAF activity. This is consistent with the supposition that GAF has an extracytoplasmic function.

D. Pseudomonas fluorescens Sequences

The disclosure provides isolated nucleic acid molecules and polypeptides from Pseudomonas fluorescens VW6. Specific nucleic acid molecules and polypeptides include:

The nucleotide sequence of Pseudomonas fluorescens DNA flanking one side of the site of Tn5 insertion at the GAF1 locus:

(SEQ ID NO: 1)

GCGCCTGACGAAGCGGCGTAGCCACAAGGAGAACCGCTGATGCGCCTGTC
CGAGCTGAAAAAGGCCGGCCGCACCCTGAGCCTGCCCCTGACCCTCGATC
TTGCGGACGCCGTCGGCCCCGGCCAGTTGCAACTGCTGAGCCTGTTGCGT
GTATTGCCGGGCGAGCGTTACGTGGGCGCGGCGGTCTGGCGCGGGCGTGC
GGTGCTGGCCAAGTTATTGGTGGGCAGCAAGGCCGCGCGGCATTTTCAGC
GTGAACTCACGGGCGTGCGCCTGCTGGCCGAACACGGCCTGACCACCCCC
CGGTTGCTCGCCGATGGCTTGCAGGAAGGCGAGGGCGGTTGGTTGCTGTT
CGAGTTCCTCGAAGGCGCCGAAAGCCTGGCCGATGCCTGGCAGGCCCGTT
CGAAGCGCTGCCGCCGCTGGCCGACGAACAAACCGCGGTGCTCGCCGAAG
CGCTGGGTGCGATCGCGCAGATGCACACCAAGGCTGTGGCAGAAGACTGC
ATCTGGACACCTGCTGCGCCAGGACGGCAAGCTGTACTTGATCGACGTG
C.

The nucleotide sequence of the 5.2 kb EcoRI fragment cloned from strain WH6:

(SEQ ID NO: 2)

CGAATTCCTTGATCTTGTGGATCTTGCCGTTTTCGCCGTAGCCGAAGAAC
GTGGTGGCGTACTCACCCTTACCGTCGATACCGAGGATCGCGGTTTTTTC
CTGGAAGCCCGAGCAGTGGTAAGCACTGGAGGCGTGGGCCAGGTGGTGCT
CAACCGGTTCGATCTTGATTTTCTTCGGATCGAAGCCCAGTTGCTCCAGG
CACCAGACGATCTTGTTGCGGTAGCGCTTGTAGCGACGGTTGCCCATCAG
GATCGCGTCGAGGGCACGGTCCGGGGCATACCAGTAACGCTTGGCGTAGT
GCCAGCGCGCCTCGCCGAACAGACTGATCGGAGCGAACGGGATCGCCACC
ACGTCAACGTCGGAAGGCTTGATGCCGGCCTGTTCCAGGCAGAACTTCGC
CGACTCGTAGGGCATGCGGTTCTTTGCATGTTTGTCGCGTACGAAGCGCT
CTTCTTCGGCGGCCGCGATCAGCTTGCCGTCGATATACAGGGCTGCGGAA
GGATCATGGCTAAGGGCGCCGGACAGGCCAAGAATCGTCAATGCCACAGG
GGTCTAGCCTCTTTTAGTCTGCATGCAGGCGGGTCGCGCCTGAAAAAAGT
GTGCTTCCCGCCTGGGCAGGAAACAGCTAAAGGGCGGGATTATAGCTTAA
AAGCAGACGAAAGCTGTTAGCGGCAAGCGGCAAGCCATTAGGAACTAGCG
GAGTTCAAGGCTGCCGTGGGCACTTTGCCGGTAGATCGTATAGGGGAGAA
TGAGCATGTCGAGGACGCCGGAGACAGGCAAGTCAAGAACGGCCCAGGGC
ACGCCGGGAATGAATCCGGTAATACGATCGTTCCCACGCGCGCGTGGGAA
CGATCAATACAGGCCTCTCACGTGAAGCACGGGGAACCATCACACTTGCC
TGGGCAACCGCTGATCAATCAGTTGGTACAACGCACTGTCCTGCGGCCAG
TTACGCATGAACCGCGCCCGATCCCTGGCATATGCCGGTGCGAAACTGGC
GGCTGAGCTGTGCTGACAGACCGAATCCAGATCAATCAGCGCCCAGCGGT
CTTCGTGCCAGAACAGGTTGTGGCCCTTGAAATCCCCATGGCTGATGCGC
TCGCCAATAAGTTGGGCAAACAGGTGGTCCAGGGCCAGCAATTCGTTTTC
CGGCGCGTCACCGTTGTCGATGTAGGGCGCAAAGCGCTCGATGATGTCCG
GGCCCGGCAGGTATTCGGTGATCAGGTAGGCACGGCTGCGCAGCCAGAAA
ACGCGCTTTTCCAATACGGCCAGTGGCTTGGGCGTGGCGATACCCAGGAA
CGCCAGGCGGTTGCCTTCACGCCAGGAGTGCCAGGCGCGGCTTGGGCGCC
AGAAGCGTTTGAGCCAATGGGCGAACCCCTTGATGTTGTAACGCTTGATC
ACCAACGGACGACCGGCCACCTCGACCTTGGCCACGCTGGCGGCACCGCC
GGTCTTGTACAGGTGGCCGCGGTCGAGCAGCGCATCGGCCTGTTCCAGCA
CCGGCAGCATCGCAGGCTCTTCTTCGCGACGAATCGCGCGCAAACCAAAC
GCACCGCGCACTACGCTGAACAGCGTGCATTCACGGCCGGCCTTGTTCAA
AAAGTCCTTCAAGCGCCAGGCACTGACCTTGCGCACCTGTTTTTCCAGCG
CTTGCAGCGGCAACGCGTGCTCACTGTTgCCGAGCAGGTAATACACCAGC
AGCTCCTCGGTAAACGGCTCGAGGTTTTTCGGCAATTGGGCGAAAAACAC
ACCGAGGTTTTCCAGTACTCGGTTGCGCGACAGCGGTTTACCGGCCTCTT
CGACACGGATGCCCGCACCGTCGATCAAGTACAGCTTGCCGTCCTGGCGC
AGCAGGTTGTCCAGATGCAGGTCTTCCTGCCACAGCCCTTTGGTGTGCAT
CTGCGCGATCGCACCCAGCGCTTCGGCGAGCACCGCGGTTTGTTCGTCGG
CCAGCGGCGGCAGCGCTTCGACGGCCTGCCAGGCATCGGCCAGGCTTTCG
GCGCCTTCGAGGAACTCGAACAGCAACCAACCGCCCTCGCCTTCCTGCAA
GCCATCGGCGAGCAACCGGGGGGTGGTCAGGCCGTGTTCGGCCAGCAGGC
GCACGCCCGTGAGTTCACGCTGAAAATGCCGCGCGGCCTTGCTGCCCACC
AATAACTTGGCCAGCACCGCACGCCCGCGCCAGACCGCCGCGCCCACGTA
ACGCTCGCCCGGCAATACACGCAACAGGCTCAGCAGTTGCAACTGGCCGG
GGCCGACGGCGTCCGCAAGATCGAGGGTCAGGGGCAGGCTCAGGGTGCGG
CCGGCCTTTTTCAGCTCGGACAGGCGCATCAGCGGTTCTCCTTGTGGCTA
CGCCGCTTCGTCAGGCGCTGCAGCCAGGTGGCGACCAGCGGCACTGTCTT
CAGGCTGGTCGAGATAAGCCGCCAGCAATTGGCGCACCTGCGCGTTCGAC
CACTGCGGTGCGCGGCGCAACAGCGGCTCCAGGTCCTTGACCCGGTCACG
CCAGCCGAACAGCAACGGGCGGGTTTTCTCCAGGTCGATCAACTGCGCGG
CGTAACCGTCACCGGGGGCCTGCAAAAAAATATGCTTGGGGTAAAAACAG
CCATGCACCTGGCCCACGCCATGCAGCCGGCGCGCCAGCCGGCCACAGGC
CAGCAGGATGGCCCGGTGTTGGGCGTCGCTCAGTTGCGGCCATTGCTCCA
GCACCGAATCCAGGTCGTTCCAGCCATCCAGGGCACGGGTCAGCAACATC
GCGCGGTGTTCGCCAGCCACCTTGCGTTCGCCATAGAACGCCGCCTGCAA
CGCCGGGATGCCCAGCGTGCGATAACGGCTGATATTGCGAAACTCGCGGG
AGAAACTCGGCTCGCCCAACGGCCGGTGCAGACTGCGGGTGAGGTAGTTG
CTCTGGCGCTTGAGGTAATACCCGTGGCCCTCTATCTCCAGGCGGAACAC
ACTGCTCCAACCGCCGCGACTGGTGTTCGGCTCGTCGACGGCGTCCAGTT
GCTTGGCCCACAGCGCGTCGAATGTCGCCAGGCCGTTACGCTCCAGCACG
GCACGGTGTTCAGCCGCCAGAAAATCACTCATTCGCGCCCCTCGAAAAAC
TTCACCACGTGGCGAATCCGCCGTTTGTCCGAATCACTCAGGTGCCGACT
GCCACGGTACTGCAAGTAGAAACGCAGGCGTTGGGTGGCCGACAGGTGAT
ACTTGGCCACTTTGTCCAGGCAAGCCAGGTCCTTGGTGATGCGGTATTTG
AGCCAGAAACCACGCCAGAAATCGCCGTTGGGACAGTCGATCAAATACAG
GGTCGACTGATCATCGACCAGCAGGTTGCGCCACTTCAAATCGTTATGGG
TGAAGCGATGATCGTGCATGGTGCGCGTGTATTCGGCGAGCTGGCGGCTG
ACCGCATCGACCCATTTGGGGTCGCGCAGGCGCGCATCGCGGTGCTCGGC
CAGCACCGAGAGGTCTTCGGTGCGTGGCAACTCGCGGGTGATCATCGCGC
CACGGTCATAGGCCAGGCCGTTACGCTCCAGGCCCCAGGCCACCACGTCG
GCGGTGGGTATGCCCCACTTGGCGAAGCGCTTGAGTTCTGCCATTCGGAC
TTGACCCGCGGCTTGCCCAGGTAGCGTCGCAGGCCCTTGCCGGCGCCGAC
GTAGCGCTTGACGTAGTAATTGACCCCACCGCGCTGCACCCGGATCACCT
CCGACAACGGGTCGCGGGTCAGGCGCTCGCCTTGCAGGGCGAACACCGCT
TCGAGGCTGCCGAAAaTCATCCGCCAGGTTGGCGTACGCAGGTTCCAGGA
TCCAACCCGCCATCAGAGCGCATCTCCATATCGCTGCTTGCGTGCGTAGA
GCTTGTCGGCCTTGGCTTGCAGCCACTGCAACAACGCGCTTTCTTCGGCC
AGGACCTGGCGCAACGGCTGCTGGAAGTAGCCCTTGAGAAAGCGCAGCTT
GTCACGGCGGGTCAGGCCGATGTCCAGCGCCGAGAAGTACAGCGCGGCCA
GGTCCTTGTCGCGCCAGCGATGACTGATCTTGTCGCGGGTCTGGGCGCGG
TGCAGGTCGATCACCGAAAGTTTGAAGTGGTCGGCGGTCACCGGCGTGTC
AGTGTGCAACAGGAAATGGCAGATGTAGCAGTCGCGATGGTTGACCCCGC
CGCGATGCATCATGCCCGTCATGCGCGCCACTTCGGCGATCAGCGCACGC
TTGAGACGTGGCTCGGGCGGCTGTTTGCGCCAGTCGATGCTGAAATCTTC
CAGGCTGACGGTGGGCGCCAACTCCTCGGTGACGATGAACGAATGCTGAT
CGGCCGGGTTGCTGGCCGCGCTCGCCATAAGCCACCGCGGTCATGGTCGG
CACACCCAATGCCTGCAAGCGCTGGATCGCCAGCCATTCCTGGCCGGCGC
CCAATACCGGGAGTTTGGCGGTGACCAGGTTCTTGACGATCTCGCCCCAG
CCGATGCCACGGTGAATCTTCACGAAATAGCCACGGCCATCCACCTCGGT
ACGCAAGGTGCGACGCGCTTCGAGTTCACGGTACACCTCGCCyTCCAGCG
CCTtCGACGGCGGCGAACGCATCGCGCCCGGCCCAGAGGGTCTTGAACGG
TTCGGCAAGCATCAACTTCATGGTTTTGGCTCCGCCAGAATCACATCCGC
AGCGTGCTGCGGCATGCTGTAGAGGTCGGCCGTCTCGGCGAAGGCCAACC
CATTGCGGCTCCAGGCCGCGCGCTGTGCAGCGTCACTCAACATACGTACC
AGGTAGCCGTTGAGCTGCGCTTGTTCAAACGGCTCATCCAGCACCAGGCC
GCTGTCGGCCTCATTGATGTAATGGGCATAACCACACACCGCCGAGACCA
GCACCGGCAGGCCGGCCACCAGGGGTTCGAGCAACACGGTGCCGGTATTT
TCGTTGTACGCCGGGTGGATCAACAGGTCGGCGCCCAGCAGGAAACGCGG
AATGTCGCTGCGTCCCTTGAGGAACTGCACGTTATCGCCCAAGCCCAACG
TGGCGCTCTGCAATTGGAATACTTTGGGGTCGTCCTGGCCGATTACAAAC
AGACGGGTGCGTTTCTTCAGTTCCGCGGGCAAGGCGGCCACAGCCTTGAG
GCTGCGGTCGACGCCCTTGGTCTTGAAGCCCGAGCCGATTTGCACCAGCA
ACAGGTCGTCATCGCCGAGGTTGAATTC.

The amino acid sequence of the ORFc polypeptide from the EcoRI fragment:

(SEQ ID NO: 3)

MRLSELKKAGRTLSLPLTLDLADAVGPGQLQLLSLLRVLPGERYVGAAVW
RGRAVLAKLLVGSKAARHFQRELTGVRLLAEHGLTTPRLLADGLQEGEGG
WLLFEFLEGAESLADAWQAVEALPPLADEQTAVLAEALGAIAQMHTKGLW
QEDLHLDNLLRQDGKLYLIDGAGIRVEEAGKPLSRNRVLENLGVFFAQLP
KNLEPFTEELLVYYLLGNSEHALPLQALEKQVRKVSAWRLKDFLNKAGRE
CTLFSVVRGAFGLRAIRREEEPAMLPVLEQADALLDRGHLYKTGGAASVA
KVEVAGRPLVIKRYNIKGFAHWLKRFWRPSRAWHSWREGNRLAFLGIATP
KPLAVLEKRVFWLRSRAYLITEYLPGPDIIERFAPYIDNGDAPENELLAL
DHLFAQLIGERISHGDFKGHNLFWHEDRWALIDLDSVCQHSSAASFAPAY
ARDRARFQV.

The amino acid sequence of the ORFe polypeptide from the EcoRI fragment:

(SEQ ID NO: 4)

MADALANKLGKQVVQGQQFVFRRVTVVDVGRKALDDVRARQVFGDQVGTA
AQPENALFQYGQWLGRGDTQERQAVAFTPGVPGAAWAPEAFEPMGEPDVV
TLDHQRTTGHLDLGHAGGTAGLVQVAAVEQRIGLFQHRQHRRLFFATNRA
QTKRTAHYAEQRAFTAGLVQKVLQAPGDLAHLFFQRLQRQRVLTVAEQVI
HQQLLGKRLEVFRQLGKHTEVFQYSVARQRFTGLFTDARTVDQVQLAVLA
QQVVQMQVFLPQPFGVHLRDRTQRFGEHRGLFVGQRRQRFDGLPGIGQAF
GAFEELEQQPTALAFLQAIGEQPGGGQAVFGQQAHAREFTLKMPRGLAAH
Q.

The nucleotide sequence of the terminal 2.4 kb EcoRI-PstI fragment cloned from strain WH6:

(SEQ ID NO: 5)

TCGCTGGTCGCCACCTGGCTGCAGCGCCTGACGAAGCGGCGTAGCCACAA
GGAGAACCGCTGATGCGCCTGTCCGAGCTGAAAAAGGCCGGCCGCACCCT
GAGCCTGCCCCTGACCCTCGATCTTGCGGACGCCGTCGGCCCCGGCCAGT
TGCAACTGCTGAGCCTGTTGCGTGTATTGCCGGGCGAGCGTTACGTGGGC
GCGGCGGTCTGGCGCGGGCGTGCGGTGCTGGCCAAGTTATTGGTGGGCAG
CAAGGCCGCGCGGCATTTTCAGCGTGAACTCACGGGCGTGCGCCTGCTGG
CCGAACACGGCCTGACCACCCCCCGGTTGCTCGCCGATGGCTTGCAGGAA
GGCGAGGGCGGTTGGTTGCTGTTCGAGTTCCTCGAAGGCGCCGAAAGCCT
GGCCGATGCCTGGCAGGCCGTCGAAGCGCTGCCGCCGCTGGCCGACGAAC
AAACCGCGGTGCTCGCCGAAGCGCTGGGTGCGATCGCGCAGATGCACACC
AAAGGGGTGTGGCAGGAAGACCTGCATCTGGACAACCTGCTGCGCCAGGA
CGGCAAGCTGTACTTGATCGACGGTGCGGGCATCCGTGTCGAAGAGGCCG
GTAAACCGCTGTCGCGCAACCGAGTACTGGAAAACCTCGGTGTGTTTTTC
GCCCAATTGCCGAAAAACCTCGAGCCGTTTACCGAGGAGCTGCTGGTGTA
TTACCTGCTCGGCAACAGTGAGCACGCGTTGCCGCTGCAAGCGCTGGAAA
AACAGGTGCGCAAGGTCAGTGCCTGGCGCTTGAAGGACTTTTTGAACAAG
GCCGGCCGTGAATGCACGCTGTTCAGCGTAGTGCGCGGTGCGTTTGGTTT
GCGCGCGATTCGTCGCGAAGAAGAGCCTGCGATGCTGCCGGTGCTGGAAC
AGGCCGATGCGCTGCTCGACCGCGGCCACCTGTACAAGACCGGCGGTGCC
GCCAGCGTGGCCAAGGTCGAGGTGGCCGGTCGTCCGTTGGTGATCAAGCG
TTACAACATCAAGGGGTTCGCCCATTGGCTCAAACGCTTCTGGCGCCCAA
GGCGCGCCTGGCACTCCTGGCGTGAAGGCAACCGCCTGGCGTTCCTGGGT
ATCGCCACGCCCAAGCCACTGGCCGTATTGGAAAAGCGCGTTTTCTGGCT
GCGCAGCCGTGCCTACCTGATCACCGAATACCTGCCGGGCCCGGACATCA
TCGAGCGCTTTGCGCCCTACATCGACAACGGTGACGCGCCGGAAAACGAA
TTGCTGGCCCTGGACCACCTGTTTGCCCAACTTATTGGCGAGCGCATCAG
CCATGGGGATTTCAAGGGCCACAACCTGTTCTGGCACGAAGACCGCTGGG
CGCTGATTGATCTGGATTCGGTCTGTCAGCACAGCTCAGCCGCCAGTTTC
GCACCGGCATATGCCAGGGATCGGGCGCGGTTCATGCGTAACTGGCCGCA
GGACAGTGCGTTGTACCAACTGATTGATCAGCGGTTGCCCAGGCAAGTGT
GATGGTTCCCCGTGCTTCACGTGAGAGGCCTGTATTGATCGTTCCCACGC
GCGCGTGGGAACGATCGTATTACCGGATTCATTCCCGGCGTGCCCTGGGC
CGTTCTTGACTTGCCTGTCTCCGGCGTCCTCGACATGCTCATTCTCCCCT
ATACGATCTACCGGCAAAGTGCCCACG6CAGCCTTGAACTCCGCTAGTTC
CTAATGGCTTGCCGCTTGCCGCTAACAGCTTTCGTCTGCTTTTAAGCTAT
AATCCCGCCCTTTAGCTGTTTCCTGCCCAGGCGGGAAGCACACTTTTTTC
AGGCGCGACCCGCCTGCATGCAGACTAAAAGAGGCTAGACCCCTGTGGCA
TTGACGATTCTTGGCCTGTCCGGCGCCCTTAGCCATGATCCTTCCGCAGC
CCTGTATATCGACGGCAAGCTGATCGCGGCCGCCGAAGAAGAGCGCTTCG
TACGCGACAAACATGCAAAGAACCGCATGCCCTACGAGTCGGCGAAGTTC
TGCCTGGAACAGGCCGGCATCAAGCCTTCCGACGTTGACGTGGTGGCGAT
CCCGTTCGCTCCGATCAGTCTGTTCGGCGAGGCGCGCTGGCACTACGCCA
AGCGTTACTGGTATGCCCCGGACCGTGCCCTCGACGCGATCCTGATGGGC
AACCGTCGCTACAAGCGCTACCGCAACAAGATCGTCTGGTGCCTGGAGCA
ACTGGGCTTCGATCCGAAGAAAATCAAGATCGAACCGGTTGAGCACCACC
TGGCCCACGCCTCCAGTGCTTACCACTGCTCGGGCTTCCAGGAAAAAACC
GCGATCCTCGGTATCGACGGTAAGGGTGAGTACGCCACCACGTTCTTCGG
CTACGGCGAAAACGGCAAGATCCACAAGATCAAGGAATTCG.

The nucleotide sequence of Pseudomonas fluorescens DNA flanking one side of the site of Tn5 insertion in strain WH6-2:

(SEQ ID NO: 6)

ATAGGCACCTACGTGCAGGCGCAACGATGAAGCCGTCTCGGCGGGTGCGA
CGTTGAAACGCGCCACATTGATGATCTGCCCGCTGTCCACCGAAGGGGCC
AGGTGATGGCAGGTCGAGCCGTAGGTTTCATCGTTGTAGTAGATCGCGTA
ATGCTGGCTGCCCAGGCCCCGGTACTTGGGCGGTGCCGGGTGCAGATTGA
TCGCGCCTTTGCGGGCATTCTTGTAGATGCTCGGCGGAAAAATGAAGTCG
CCACGGTAGGAGATGATCCAATCGCCTTCCCAGTTATCCAGATGGTAAGG
ATAGGGATCGCCCGGGTCCCAGCAAAACACCTCAAGGTTGGAGAATACGG
TCTTGGCGAATTCACGCCGTGATCGCACCAGTCCATGGTGCACACCGACA
GCAGGACCTTGTCTTGCATGGCTCGATTGTCTGCATCTTACTTNTTCATT
TCAGGTAATGTGGGTGACAACGCCCGCCCCCCGTCAAAGCCCGCCTTGTC
GCTAGTACGCGACAGCCTTA.

The nucleotide sequence of the 5 kb BamHI fragment cloned from strain WH6:

(SEQ ID NO: 7)

GGATCCCGAAGTTCGCTACTTGCCCAACGGTAACGCCGTGACCAACCTGA
GTCTGGCGACCAGCGAACAGTGGACCGACAAGCAGACCGGCCAGAAGGTC
GAGAAGACCGAATGGCACCGTGTGTCGATGTTCGGCAAGGTTGCAGAGAT
CGCTGGTGAGTACCTGCGCAAAGGTTCGCAGGTCTACATCGAAGGCAAAC
TGCAAACCCGCGAGTGGGAAAAAGACGGCATCAAGCGTTACACCACCGAG
ATCGTGGTCGACATGCAAGGCACCATGCAACTGCTGGGCGGCCGTCCACA
GGGCGACCAACAGGGGCAGGGTGGCATGTCCAACTCGGCACCGCGTCCAC
AACAGTCGCGTCCACAGCCAAGCCAGCAGCCACAGCGTGAGTCGCGTCCA
GCGCCCCAGCAGGCCGCTGCGCAACCGGCGCCGGATTTCGACAGCTTTGA
TGACGATATTCCGTTCTAACGCAGCTTTCAGTACCGTAACCCATAAAAAA
GCGAAGCCATTAATCTGGCTTCGCTTTTTTTATGGCCACAGATTTTTGTG
AAGTAACAACTGCGTGCCTGGGATCAACTCGTATGCTGCAGCAGCGGATA
CAGCGAAATCACCAGCAACGCCGCCATGCCGAAATTGAACATGCGCAGCC
AGCGCGGGTTGCGCAGCACGTTGCGCAGTACACTGCCGAAnCCCGCCCAT
ACCCCCACGCTTGGGGCGTTGATCAGAGCGAACACCGTAGCGATGATCAC
CACGTTCaTGGTGTAGCCCTGCaAGGGCGTGTACGTGCTGATCGCACCAA
TTGCCATGACCCACGCCTTGGGATTGACCCACTGAAACGCCGCTGCGCCC
CAGAACCCCAGGGGCTTGCCTTTACCTTCGGTGTCTTCTGATGCCGGGCC
GGAACGGGCGATGTTCCATGCCAGGTACAGCAAGTAAGCCGCGCCCGCAT
AGCGCAGCACGGTGTAGAGCACCGGATAGGCGGTAAAGGCTGCACCCAGC
CCAAGCCCCACGGCCAATACCAGCACCAGGAACCCGGAGCTGATACCCAG
GATGTGCGGGATGGTGCGCTGGAAGCCAAAGTTGACGCCCGAGGCCAACA
GCATGGCGTTATTGGGGCCAGGGGTGACGGAAGTGACAAACGCAAACAAA
GCAAACGCCAGCATTAAATCAAACGACAAGGACATGGATATTTCCTGGAA
ACGGGCCTTCGCGCTTACCTGCCACCCGGACAGCGCGGGATACAGGTGCA
GTGTGGCCACTTGTAGTTGGAGTCAGAACAACGAACTGAGAAAACCAATG
GCAACCATCGAGTAGAACGCTGCACAAATCAGCCCGAAGGACTTGCGCGT
CCGGGTGaAAAACGGTTCTGCCGAATGGGCGGAAAACGCTAAGGCGTAGG
TACAGAAAATCAGGAAACCCAATAGCGCACAGGCCGCCACCAGCACGAAA
CTGGCCCAGGCCGGCGCGTTGACGCTCAGGCCCACGGTGGTCACCGTGAA
CCAGGTCAAGGCCGCCTTGGGGTTAGTCAGGTGAATGCCCAACCCCTGCA
AATAGAAGCCCAGGTTGCGCGTCTGCTTTTCCCGTACTTTGGCTACCGTC
GTGGCGTTGTGCGCCAGCGCACTGCGCACCGATTTCCAGGCCAGGAAAAA
CAGATAGCAGGCGCCGAACACTTTTAACCACAGGATGATCTGGGTGTTGG
ACATCAACAACGCCGACACGCCTGCGGCGGTCATCGCGCCCCAGCACAAC
GACCCGGAAATCACCCCGGCCGCCAACGCCAGGCCCGGTGTGCGCCCGTA
GTTCAAGGAGGTGTTGGCGATGGCCAGGTTGCCCGGGCCAGGGCTGGCGG
TGCCGATCACGTAGACCCCCAGTGCCGCTGCAAAACTCGAAAACTCAAAC
ATAGATGCACTCATACCCGTCGCGCCAGCGCGCAGTGAGCACTTCAAGCC
ACCCATGGGTTGAAGTGACGGCCAAGAAAAAGCTATTTGAAGCAACGATG
ATCCGGCTCCTGGGCGCGGATCTTCTCCATCCAGGGCTTGAGTTCCGATT
GCTTGTACAGGCGCTCGCCCCAATTTTCCGGCGAGACCGGCAGTGGCCGT
CCCAGCAGGATGTAGTCGGTGAGCAGGTGGATGACTGCTGCAGGCAATAG
GCACGTACGTGCAGGCGCAACGATGAAGCCGTCTCGGCGGGTGCGACGTT
GAAACGCGCCACATTGATGATCTGCCCGCTGTCCACCGAAGGGGCCAGGT
GATGGCAGGTCGAGCCGTAGGTTTCATCGTTGTAGTAGATCGCGTAATGC
TGGCTGCCCAGGCCCCGGTACTTGGGCGGTGGCGGGTGCAGATTGATCGC
GCCTTTGCGGGCATTCTTGTAGATGCTCGGCGGAAAAATGAAGTCGCCAC
GGTAGGAGATGATCCAaTCGCCTTCCCAGTTCCAGATGGTAAGGATAGGG
ATCGCCCGGGTCCCAGCAAAACACCTCAAGGTTGGAGAATACgGTCtTGG
CGAATTCCACGCCGTGATCGCACCAGTCCATGGTGCACACCGACAGCAGG
ACCTTGTCCTTGCATGGCTCGATTGTCTGCATCTTcACTTcTCATTTcCA
GGTAATGTGGGTGACAAcGCCCGcCCCCCCGtCAAGCCCGCgcTGTCGCT
AGTACGCGACAGcCCTgACCACTGCGTTGAAGCGTTCGACCATTTGCCCC
AGCTCCCCTTCGCTGGTGATAAACGGCGGTGCAAACAGCACATGATTGCC
GTGCGTTCCCTGCACGGTGCCGCTGCCGGGATAGATCAACAGGCCGCGCT
GCAACGCCTCTTGCTTGAGCGCCGCGGCATACGCGCCGCCGCCCTTGAAC
GGCGCGTTGGTGGCGCGGCTCTCGACAAACTCGACGCCCACGAACAGCCC
GCGCCCGCGCACATCACCGACGATATCCAGGTCAGCCAGGCTTTCCCTGA
GCCAGGCGCGCAATTGCTCACCGCGTTGGCGCACTTGCGCCAGCAGGTCT
TCGTCCTCGATGACTTGTTGCACTTCCAAGGCAATGGCACAGGCCAGCGG
gATGGTTGACGTGGGTCTGGCCGTTGCCCAGCACGCCTGAACCACGGGCC
ATTACCGAGTGCACCTGGTCGCTGATCAACAGTGCCGAGATCGGCATGTA
GCCCGCCGCCAGGCCTTTGCCGACCGCGACCATGTCCGGCACGATGCCGT
CGTCGGCATAGGcGAACAGCTGCCCGGTACGCCCCATGCCGGCCATCACC
TCATCGAGGATCAGCAGCACATCGTGACGCTCGCACACGGCCTTGATCTT
GCGAAAGTAACCGGGCACCGCAGGCACCGGGCCATTGGTGGAGCCCACCA
CGGTTTCGGGAAAGAACGCCGCGACGTTTTCACTGCCCAGGCTGCGAATT
TTGGCGTCCAGTTCATCGGCCAGGCGCGTGGCGTACTGCTCTTCGCTTTC
GTCGGGCCGTTGATCACGGTAGGCGTAGCATGGCGAGACAAACTCGGCCG
CCGCGAACAGTTGACCAAACACCGATTGGCGCTGCGGGTTACCGGAGATG
CTCAAGGTGCCCAGGGTGCTGCCGTGGTAGCTCTGCCGGCGGGAAATGAA
CAACGACTTTTCAGGCAAGCCGCGTTCGCATTGGTACTGGTAAGCGATTT
TCATCGCCAGCTCCATCACCTCCGAGCCGCCGGATAAAAACTGCGCACGC
GCCAGGCCGCCACTGGCTGCCACCAGGCGTTCGGCCAGTTCCTCGGCGGC
GGCGGTGGTGAAGCTGCCCGCGTGCGCCCACGCCAGTTTCTTCGCTTCGC
GCTCGAATGCCGCTGCGATACGCGGATGGCCATGGCCCAGGCTCGACACC
GCGACACCGCCGCAGGTGTCGAGGTAGCGTTTGCCCGTTGCATCCTCAAG
CCACACCCCTGCGCCGCCGACCGCCAGGATCGGCGAGGTGTTGAGATTTT
TGTAAATCACTTTTGACATGCGCGCGGTTCCTTGGGCGATTGGCCTTACG
GGTTACTTACGGAGTGATCCCAAGCCGTCGGCGCCTGGTGAAAGCGCCGC
CCGAAACGGGACGAATGAAACACCGAACGCGTCAGCCCGATGGGCAGCAA
CACAACGATAAACATCAAAAACGCAAGAAATCTtCAtGCAgCGGTAAAAG
CTCCAAAGAGTGAAaGGGGGTGGGGATGCAGTCCCAGCcGGGATGCTACG
CTGCGTCCAGTCAACGGCCTCGACGAAGGCCGGGTGGGCGGGgCGCTCAA
GCACGTAGCGGCCCAGAACCAGCACGTCCATTTCCGTGCGCATGAAGCAG
GCGTACGCTTCCTGCGGCGTGCACACGATCGGTTCGCCACGCACGTTGAA
CGAGGTGTTGACCAACACCGAGCAGCCGGTGTGCTCCTTGAAACTGCGCA
GCAGGTAGGTGAACGGTGCGTTGTTTTCTTCGGTCACCGTTTGCACCCGC
GCCGACAGGTCGACGTGGGTCACCGCCGGCAACTGCGAGCGAATATGGTT
GATGCTGCCCAAGCCCCGCTCGGGGCCGCCTTTCTTGCAGCGACTGCTGG
GATTTGATCGAGTCGGTGACCGGCGCGACCATCAACATGTACGGGCTTTT
TTCGCAGATATCGAAATAGTTGCCGGCGTCTTCGGCCAGCACGGCCGGGG
CGAACGGGCGGAACGACTCGCGGTATTTGATCTTCAGGTTCATGGTGCGC
TGCATCTCGGGGTTGCGCGCATCGCCGAGAATCGAACGCGCGCCCAAGGC
CCGTGGGCCAAACTCCATGCGCCCCTGGAACCAACCCACCACCGCAGCGT
CGGCCAGGCGACTGGCGACCTGGTCATACAGCCCGGCGTCGTGGTAGCGG
GTAAAGGGGTAGTGGTTTTCCAGCAGGAACTGGGCGATTTCTTCATCGCC
AAAACCCGGCCCCAGCAGGCTGCCGCTCATGGCATCGGACTTGTTGCCAT
CCAAGTGCGGGCGACCGCTGTGCTTGACCGCAAAATCCAACGCCGCCCCC
AGGGCACAACCGGCATCGCCGGCCGCCGGCTGGATCC.

The amino acid sequence of the ORFb polypeptide from the BamHI fragment:

(SEQ ID NO: 8)

MQTIEPCKDKVLLSVCTMDWCDHGVEFAKTVFSNLEVFCWDPGDPYPYHL
DNWEGDWIISYRGDFIFPPSIYKNARKGAINLHPAPPKYRGLGSQHYAIY
YNDETYGSTCHHLAPSVDSGQIINVARFNVAPAETASSLRLHVGAYCLQQ
FIHLLTDYILLGRPLPVSPENWGERLYKQSELKPWMEKIRAQEPDHRCF
K.

The nucleotide sequence of Pseudomonas fluorescens DNA flanking one side of the site of Tn5 insertion in strain WH6-3:

(SEQ ID NO: 9)

GCCCAGGACAGCGGCACCATGCAGGCCTGGCTCGACCGCTATTTCAACCA
GGCCCATCGCCTGCCGGATTTGAGCCCATCGGGGTTCAAGCCGGTCAGCG
GGCGTTTGCTCAGCACCGAGCAAGGCGCTGCCGCCATGGTGCTTTACCAG
GACGCGCAAGGCCGGCGCATCAGTTTCTATATCCGGCCGCCGGGGCCGAA
CAACGGTTTTCTACCGCGTGGCAGCCGCACCGCAGATGGGCTGCAAGCGC
AATACTGGTCCGGCGGCGGCTACAACTATGCGGTCGTCAGCCCGGCGGAC
CAGGTGCCCGCACACCTGTTGCAGTTCTAGGCGCGGCGCTCCTGTATTGG
TCAAATCAGCGCGAGCGCCCTAAAGTGAGCGACCACCGCACTACAGAGTG
ACTCGCATTGGCCGTGCTTCCCTGCTCCGTCGTTACTCGGTAAACACAGC
GCCCCACGACACCTCGCCATCAGGCATTCTTTGAGAACCAGGCCCGGCAA
CAGGTTACACCTcAGCGCGCAGTCCGGCATCGCCGCATGACGCGAACCG.

The nucleotide sequence of the 3.4 kb PstI fragment cloned from strain WH6:

(SEQ ID NO: 10)

CTGCAGGCGTTCGATCAACTCAACACCGAACAACGCGCGTTGTTGCTGTG
GGTCTCGGTCGAAGGCTTGAGCTACAAGGAAGTCGCCGAAATACTCGACG
TGCCGCTTGGCACCGTGATGTCACGCCTGTCCCGCGCCCGCCAAGCCCTG
CGGCAACTCAGCGACGGCGAAATTGCCAGCCCTTCCCTGCGGATACTCAA
ATGATCAGCCTGCCCCCCAGCGAACGCGACCTGCATGCCTACGTCGATCA
CCAACTCCTGGAGAGCGATCGCCGTGTTCTCGAAACGTGGCTGACCGCGC
ACCCCGACGTCGCGGCCCAAGTGCATGCCTGGCAGCAGGATGCGCAGTTG
CTGCGCGCGTCATTGAGCGGCGCCCTGCAACAGCCCGCCAACCCCAACCT
TGACCCGGCGCTGATTCGCCAGCGCATCCAGCACCGGTCGCGCCGCCACT
TCGCCACGGCCGCCGTGTTGCTGATCGCCGTGAGCCTTGGCGGCCTCGGC
GGCTGGCATGCCCGTGAAGCCACGCAATCACCCCAACAGCCAATGGCCGA
CGCGATGCAAGCGTTCCGGCTGTTTGCCCAGGACGGCATCCTGCCCGCCG
ATTACAACGCCCAGGACAGCGGCACCATGCAGGCCTGGCTCGACCGCTAT
TTCAACCAGGCCCATCGCCTGCCGGATTTGAGCCCATCGGGGTTCAAGCC
GGTCAGCGGGCGTTTGCTCAGCACCGAGCAAGGCGCTGCCGCCATGGTGC
TTTACCAGGACGCGCAAGGCCGGCGCATCAGTTTCTATATCCGGCCGCCG
GGGCCGAACAACGGTTTTCTACCGCGTGGCAGCCGCACCGCAGATGGGCT
GCAAGCGCAATACTGGTCCGGCGGCGGCTACAACTATGCGGTCGTCAGCC
CGGCGGACCAGGTGCCCGCACACCTGTTGCAGTTCTAGGCGCGGGCGCTC
CTGTATTGGTCAAATCAGCGCGAGCGCCCTAAAGTGAGCGACCACCGCAC
TACAGAGTGACTCGCATTGGCCGTGCTTCCCCTGCTCCGTCGTTTACTCG
GTAAACACAGCGCCCCCCACGACACCTCGCCCATCCAGGCATTCTTTGAG
AACCAGGCCCGGCAACAGGGTTACACCCTCAGCGCCGGCCAGGTCCGGGC
AATCGCCGCCATGGCACGCGAAACCCGGCACCGGCTCGCCCGCCGGCCCA
CTCGAAGCCTGTACCTGCACGGTCCGGTGGGACGTGGCAAAAGCTGGCTG
CTGGACGGTTTCTTCCAGGCGCTGCCCATCGCCGAGAAACAGCGTGTGCA
TTTCCATGAGTTTTTCGCGCGCTTGCACCGCGGCATGTTCGCCCACCGGG
CCGTGGCACGATGCGCTCGCCATGACCCTCGATGAGTTGCTCGACGGCTG
CCAGGTGCTGTGTTTCGACGAGTTCCACGTCCACGACATCGGCGACGCCA
TGCTGATCACGCGGCTGTTCCAGGGCCTGTTCCGGCGCGGTGTCTGGGTG
CTGGTCACCTCCAACTATCCGCCTGAAGGCCTGTTGCCCAACCCTCTGTA
CCACGAGCGTTTCAAGCCGGTGATCGACTTGATCGCCGCGCGCATGGACG
TCTTGGAAGTCAGCTCCCCCGAGGATTTTCGCCGCTTGCCCCAGGCCCAC
GCCACGCAACGCTTTACCACGGGGCAGTATGTGTGGCCCGGCACCGCGGC
CCAACGCGCGACGCTCGGCCTCCCCGCCACGGATGGCCCGGCGCATACCC
TGGCAGTGGGCAACCGGCAGTTGGCCTGCCGCTACCATCAGGCGCGAACG
GTCGCGTTCACCTTCAGCGACCTCTGCGAACAACTGACGGCCGTGATGGA
TACCTGCTGCTGTGCGAAGATTTCGACCACTGGATCATCGACGGCCTGCC
TCACCTGGCCGAGTGTCCGATTGCCGTGCAGCAGCGCTTTATCAACCTGG
TGGACGTGCTCTACGACAAAGACAAGCACCTGGTGCTGATCGGTGAACAG
CCACTGGCACAGGCGATGAGTGGCGAGGCCATCGACCTCGCCCGCACCGC
CAGCCGCCTGAACCAATTGCAACAGGCCAGCCCGCAACCGGTGCCCGACC
CGGTATCATGAGCGCCCTTTACGCCCCCTAGCCGAGTGACCGCGCCGCTG
ATGCATACCCTCGCCCAACTCAAGTCCGGCCAATTGGCCGGTATCCAGCG
CCTGGACCTGTCCTGCGGGCTCACCGAATTTCCCCGGGAAATCTTCGAAC
TGGCCGAGTCCCTGGAGATCGTCAACGTCAGCGGTAACGCCCTGAGCCGG
TTGCCCGACGACCTGCATCGCCTGCCACACCTGCGGGTACTGTTCTGCTC
GGACAACGCCTTCACCGAACTGCCCGAATGCCTGGGCCAGTGCGCGCAAC
TGAGCATGATCGGTTTCAAGGCCACCAGATCAGCCACGTGCCCGCCGCCG
CGCTGCCGCCGTTGCTGCGCTGGCTGATCCTCACCGACAACCGTATCAGC
CAATTGCCCGATGAATTGGGCGAGCGCCCACTGCTGCAAAAACTGATGCT
GGCCGGCAACCAATTGGCGCATCTGCCGCCGAGCCTGAGTCACTGTCACC
ACCTCGAATTGCTGCGCATCGCGTCCAACCGCTTCACCCAGCTGCCGGCG
TGGCTGTTGACCCTGCCCAGCCTGACCTGGCTGGCCTATGCCGGTAACCC
GGTGGAAATGGCGGTGGATGTAGCGGTGGACGACGCCACGCCCGACATTC
CTTGGGCCGAGCTGGAACTGGCCCAAGTGCTGGGCGAAGGCGCTTCGGGG
GTGATTCGCAAAGCGCTGCGTAAACCCACGGGCACGCCCGTTGCCGTCAA
GCTCTACAAAGGCACCATCACCAGCGACGGTTCGCCGTTGCACGAAATGC
AAGCCTGCATCGCCGCCGGGCTGCACCCCAACCTGATCAGGGTGCAGGGC
CGCGTCATCGGCCACCCCGATGACCAGGCTGCGCTGGTGATGGACCTGAT
CGACCCGAGCTACCGCAACCTGGCGGCCCTGCCGAGCCTGGCCTCGTGTA
CCCGTGACATCTACGCGCCCGACACGCGTTTCAGCGCCAAAGTGGCCTTG
CGTATGGCACGGGGCATCGCCTCGGTGGCCGCGCATCTGCACCGGCACGG
CATTACCCATGGCGACCTCTACGGCCACAACATCCTGTGGAATGAAGCCG
GGGATTGCCTGCTCGGGGATTTTGGCGCGGCGTCGTTCCATGCCACGGCG
GATACCCTCGAAACACGGGCGCTGCAG.

The amino acid sequence for the putative PrtI polypeptide encoded by strain WH6:

(SEQ ID NO: 11)

LQAFDQLNTEQRALLLWVSVEGLSYKEVAEILDVPLGTVMSRLSRARQAL
RQLSDGEIASPSLRILK.

The amino acid sequence for the putative PrtR polypeptide encoded by strain WH6:

(SEQ ID NO: 12)

MISLPPSERDLHAYVDHQLLESDRRVLETWLTAHPDVAAQVHAWQQDAQL
LRASLSGALQQPANPNLDPALFATAAVLLIAVSLGGLGGWHAREATQSPQ
QPMADAMQAFRLFAQDGILPADYNAQDSGTMQAWLDRYFNQAHRLPDLSP
SGFKPVSGRLLSTEQGAAAMVLYQDAQGRRISFYIRPPGPNNGFLPRGSR
TADGLQAQYWSGGGYNYAVVSPADQVPAHA.

The amino acid sequence of the ORFc polypeptide from the BamHI fragment: MVERFNAVVRAVAY (SEQ ID NO: 13).

TABLE 13
GAF activities recovered from culture filtrates of wild type
Pseudomonas fluorescens strain WH6 and three
Tn5-induced mutants

| | | MEAN GERMINATION SCORE* | | |
|---|---|---|---|---|
| | | (±Standard Error of the Mean) | | |
| | | AT RELATIVE SAMPLE | | |
| PSEUDOMONAS | | CONCENTRATION (X) INDICATED | | |
| STRAIN | MUTATION | 0.1X | 0.3X | 1.0X |
| WH6 | | 1.1 ± 0.0 | 1.0 ± 0.0 | 1.0 ± 0.0 |
| WH6-1::Tn5 | gaf1 | 4.0 ± 0.0 | 4.0 ± 0.0 | 4.0 ± 0.0 |
| WH6-2::Tn5 | gaf2 | 4.0 ± 0.0 | 4.0 ± 0.0 | 3.8 ± 0.1 |
| WH6-3::Tn5 | gaf3 | 4.0 ± 0.0 | 4.0 ± 0.0 | 3.7 ± 0.1 |

Aliquots of culture filtrates from the wild type strain WH6 and the three Tn5-induced mutants were tested in the Standard GAF Bioassay System as described in Example 1.
*Germination Score of 1.0 = Germination completely arrested immediately after emergence of the coleorhiza and plumule. Germination Score of 4.0 = Normal germination and seedling development. (See Table II-1 for details of the scoring system.)

TABLE 14
Restoration of GAF activities in culture filtrates of Pseudomonas
fluorescens mutants transformed with wild-type genes

| | | MEAN GERMINATION SCORE* | | |
|---|---|---|---|---|
| | | (±Standard Error of the Mean) | | |
| | | AT RELATIVE SAMPLE | | |
| PSEUDOMONAS | | CONCENTRATION (X) INDICATED | | |
| STRAIN | MUTATION | 0.1X | 0.3X | 1.0X |
| WH6 | — | 1.1 ± 0.0 | 1.0 ± 0.0 | 1.0 ± 0.0 |
| WH6-1::Tn5 | gaf1 | 4.0 ± 0.0 | 4.0 ± 0.0 | 4.0 ± 0.0 |
| WH6-1::Tn5 | | 1.6 ± 0.1 | 1.1 ± 0.1 | 1.0 ± 0.0 |
| (pOSU5200) | | | | |
| WH6-1::Tn5 | | 2.1 ± 0.1 | 1.0 ± 0.0 | 1.0 ± 0.0 |
| (pOSU2400) | | | | |
| WH6-2::Tn5 | gaf2 | 4.0 ± 0.0 | 4.0 ± 0.0 | 3.8 ± 0.1 |
| WH6-2::Tn5 | | 1.6 ± 0.2 | 1.0 ± 0.0 | 1.0 ± 0.0 |
| (pOSU5000) | | | | |
| WH6-3::Tn5 | gaf3 | 4.0 ± 0.0 | 4.0 ± 0.0 | 3.7 ± 0.1 |
| WH6-3::Tn5 | | 1.0 ± 0.0 | 1.0 ± 0.0 | 1.0 ± 0.0 |
| (pOSU3400) | | | | |

Aliquots of culture filtrates from the wild type strain WH6, the three Tn5-induced mutants, and the mutants transformed with the corresponding wild type gene, were tested in the Standard GAF Bioassay System as described in Example 1.
*Germination Score of 1.0 = Germination completely arrested immediately after emergence of the coleorhiza and plumule. Germination Score of 4.0 = Normal germination and seedling development. (See Table II-1 for details of the scoring system.)

### Example V

### Physical and Chemical Characteristics of the Germination-Arrest Factor (GAF) from Pseudomonas fluorescens WH6

Summary

The Germination-Arrest Factor (GAF) produced by Pseudomonas fluorescens WH6 is a hydrophilic molecule that is insoluble, or sparingly soluble, in all organic solvents tested to date. It has a molecular weight that is less than 3,000 daltons and probably less than 1,500 daltons. It contains at least one ionizable group. GAF reacts with ninhydrin, and it can be visualized as a ninhydrin-positive band that chromatographs with characteristic Rf values in defined thin-layer chromatography systems. The identification of GAF as the compound responsible for the specific ninhydrin-positive band in question has been accomplished by mutational genetic analysis.

A. Solubility

The Germination-Arrest Factor (GAF) from Pseudomonas fluorescens WH6 culture filtrates is a water-soluble compound that has very hydrophilic characteristics. It is insoluble in organic solvents that are immiscible with water (e.g. ethyl acetate, chloroform) as shown in Tables 15 and 16, and it is insoluble or only sparingly soluble in more polar organic solvents (e.g. acetonitrile, absolute ethanol, isopropanol, methanol) as shown in Table 16. The addition of water to ethanol greatly increases the solubility of the active compound in this solvent, and extraction of dried culture filtrates with 90% (v/v) ethanol under carefully controlled conditions has been used as a preliminary purification step to lower the amount of inorganic salts associated with the active compound and permit concentration of the compound for more extensive biological testing and for chromatographic purification. The recovery of the active compound from dried culture filtrates by extraction with varying concentrations of aqueous ethanol solutions is illustrated in Table 17.

B. Molecular Weight

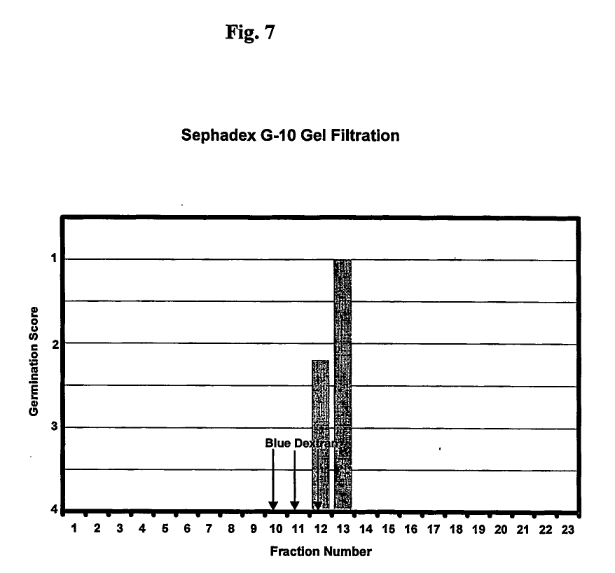

GAF from Pseudomonas fluorescens WH6 culture filtrates has been shown by ultrafiltration to have a molecular weight less than 3,000 daltons (Table 18). GAF elutes from Sephadex G-15 gel filtration columns at a position slightly later than the Blue Dextran marker used to measure the void volume of the column (FIG. 7). This behavior is consistent with a molecular weight less than 1,500 daltons, which agrees with the fractionation of GAF activity observed using a ultrafiltration membrane with a molecular weight cut-off of 1,000 daltons (Table 18).

C. Ionic Properties

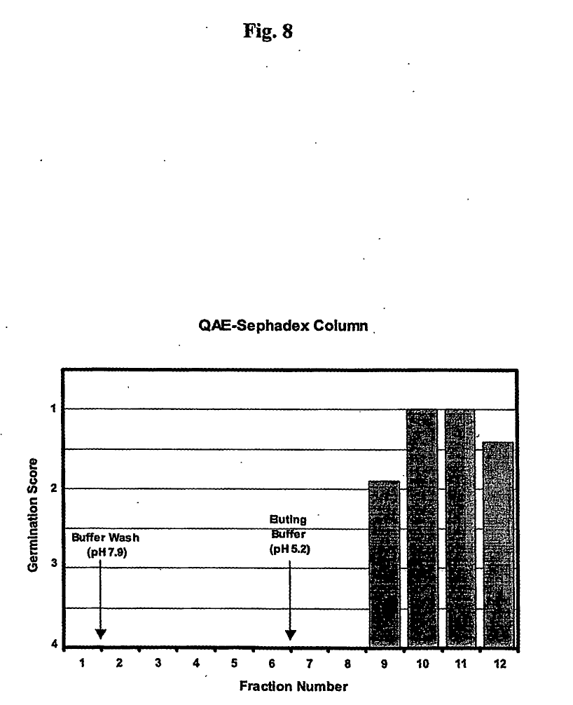

GAF from Pseudomonas fluorescens WH6 culture filtrates appears to have at least one anionic group. This conclusion is based on the pH-reversible binding of the active compound to QAE-Sephadex anion exchange columns. As shown in FIG. 8, GAF binds to QAE-Sephadex columns at pH 8.0 and is eluted from the column when the pH of the eluting solution is adjusted to pH 5.6. This behavior is consistent with the presence of an anionic group with a pK between 5 and 8.

D. Stability

The GAF activity associated with Pseudomonas fluorescens WH6 culture filtrates appears to be stable when stored under sterile conditions at 4° C. or at room temperature. GAF activity is not measurably affected by heating to temperatures up to 55° C. for periods of at least 1 hour, but some loss of activity is evident at 75° C., and activity is destroyed in boiling water (100° C.) or by autoclaving (Table 19).

The GAF activity associated with WH6 culture filtrates is not affected by exposure to acid pH (down to pH 2) for periods of up to 3 hours (Table 20). Similarly, GAF activity does not appear to be affected by exposure to weakly alkaline pH (up to pH 9) for 3 hours, but exposure to more extreme alkaline pH values (pH 11 and above) results in some lost of activity (Table 20).

E. Choromatographic and Chemical Properties

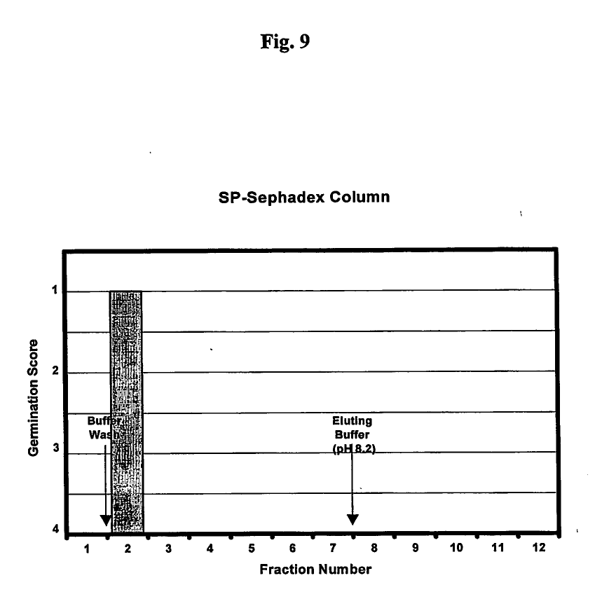

GAF does not bind to the Silica-C₁₈ columns which are frequently used for the trace enrichment and fractionation of hydrophobic compounds (Table 21). As indicated previously, GAF does bind reversibly to the anion exchanger QAE-Sephadex at alkaline pH (FIG. 8), but it does not bind (or is only slightly retarded) when applied to a column of SP-Sephadex (a cation exchanger) at pH 4 (FIG. 9).

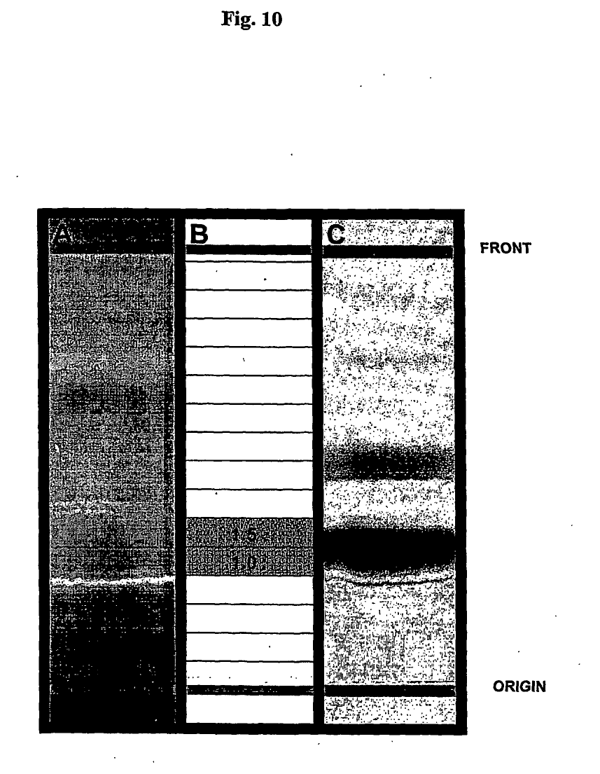

Thin-layer chromatography (TLC) on microcrystalline cellulose plates has been used to separate the biological activity associated with GAF from a number of contaminating ultraviolet-absorbing and ultraviolet-fluorescing compounds (FIGS. 10A and B). On these TLC plates, using ethyl acetate:isopropanol:water (15:30:20) as the solvent, GAF activity consistently co-chromatographs with a ninhydrin-positive band which has an Rf value less than 0.5 (FIGS. 10B and C). Other ninhydrin-positive bands are present on the chromatogram but are not associated with any detectable biological activity. As expected, GAF activity recovered from the other bacterial isolates identified in Section I also chromatographs with a ninhydrin-positive band of approximately the same Rf.

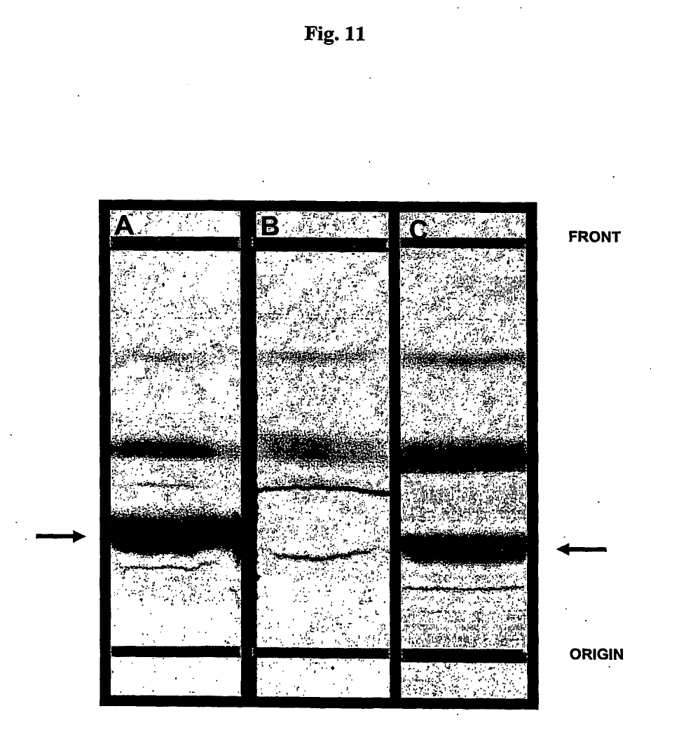

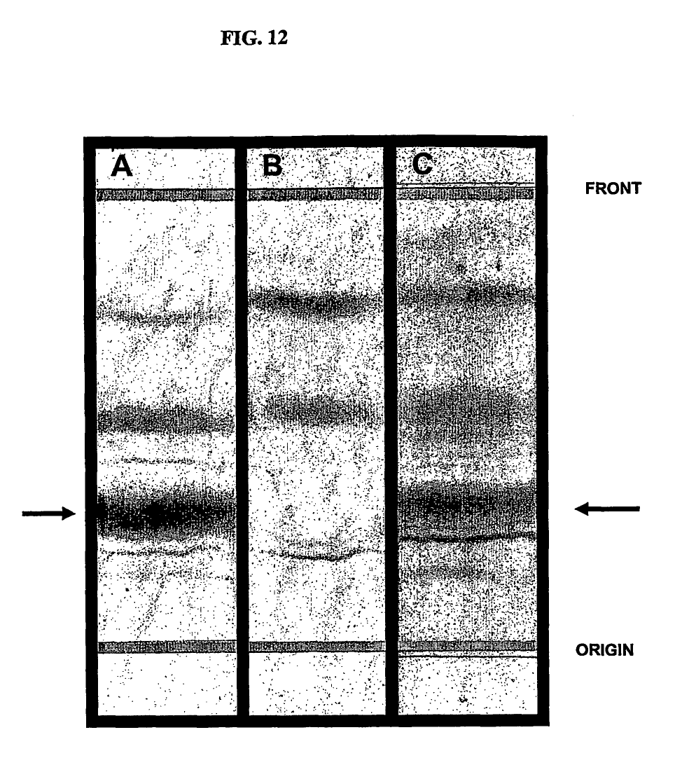

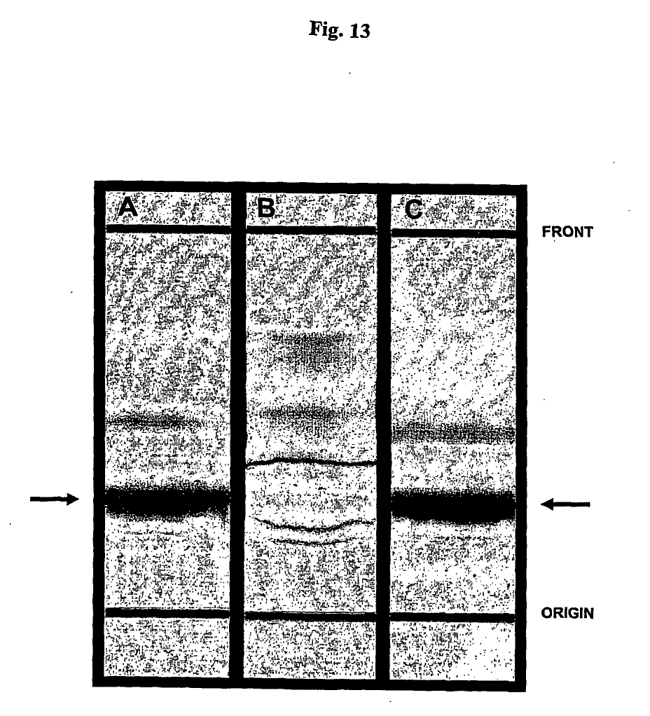

The identity of GAF with the ninhydrin-positive band with which GAF activity co-chromatographs in TLC separations has been demonstrated using the three mutant strains of Pseudomonas fluorescens WH6 (WH6-1, WH6-2, WH6-3) generated by transposon mutagenesis as previously described. Coincident with the loss of biological activity in each of the three GAF mutants, the ninhydrin-positive band that co-chromatographs with GAF activity in the wild type disappears (FIGS. 11A and B, 12A and B, and 13A and B). This specific ninhydrin-positive band reappeared when the biological activity of GAF1, GAF2, and GAF3 was restored by complementation (FIGS. 11C and 13C). This result establishes the identity of GAF with the ninhydrin-positive band in question. (Complementation of GAF2 is identical to the results shown for GAF1 and GAF3.) The plasmids used in complementation studies are presented in Table 14.

TABLE 15
Distribution of GAF activity in aqueous and organic phases
resulting from partition of WH6 culture filtrate against
organic solvents that are immiscible with water

| | | MEAN GERMINATION SCORE* | | |
|---|---|---|---|---|
| | | (±Standard Error of the Mean) | | |
| | | AT RELATIVE SAMPLE | | |
| | PARTITION | CONCENTRATION (X) INDICATED | | |
| SOLVENT | PHASE | 0.1X | 0.3X | 1.0X |
| Chloroform | Organic | 4.0 ± 0.0 | 4.0 ± 0.0 | 4.0 ± 0.0 |
| | Aqueous | 1.3 ± 0.1 | 1.0 ± 0.0 | 1.0 ± 0.0 |
| Ethyl Acetate | Organic | 4.0 ± 0.0 | 4.0 ± 0.0 | 4.0 ± 0.0 |
| | Aqueous | 1.3 ± 0.1 | 1.0 ± 0.0 | 1.0 ± 0.0 |
| Culture Filtrate | — | 1.7 ± 0.1 | 1.1 ± 0.1 | 1.0 ± 0.0 |
| Control | | | | |
| Aliquots (30 ml each) of Pseudomonas fluorescens WH6 culture filtrate were mixed with 60 ml of either chloroform or ethyl acetate. The aqueous and organic phases were shaken vigorously in a separatory funnel for 3 minutes. The phases were then allowed to separate, and the aqueous and organic phases from each partition were taken to dryness separately in vacuo at 45° C. The solids recovered from each phase were dissolved in separate 30 ml | | | | |
| # aliquots of distilled water and tested for GAF activity in the Standard GAF Bioassay System (see Example 1) using seeds of annual bluegrass (Poa annua). | | | | |

*Germination Score of 1.0 = Germination completely arrested immediately after emergence of the coleorhiza and plumule. Germination Score of 4.0 = Normal germination and seedling development. (See Table 5for details of the scoring system.)

TABLE 16
Solubility of WH6 GAF activity in various organic solvents

Mean Germination Score*
(±Standard Error of the Mean)
at relative sample
concentration (X) indicated

| SOLVENT | FRACTION | 0.1X | 0.3X | 1.0X |
|---|---|---|---|---|
| Acetonitrile | Extract | 4.0 ± 0.0 | 4.0 ± 0.0 | 4.0 ± 0.0 |
| | Residue | 2.0 ± 0.1 | 1.0 ± 0.0 | 1.0 ± 0.0 |
| Acetone | Extract | 4.0 ± 0.0 | 4.0 ± 0.0 | 4.0 ± 0.0 |
| | Residue | 1.8 ± 0.1 | 1.2 ± 0.0 | 1.0 ± 0.0 |
| Chloroform | Extract | 4.0 ± 0.0 | 4.0 ± 0.0 | 4.0 ± 0.0 |
| | Residue | 1.7 ± 0.2 | 1.2 ± 0.1 | 1.0 ± 0.0 |
| Ethanol | Extract | 4.0 ± 0.0 | 4.0 ± 0.0 | 4.0 ± 0.0 |
| (Absolute) | Residue | 1.8 ± 0.1 | 1.1 ± 0.0 | 1.0 ± 0.0 |
| Ethyl | Extract | 4.0 ± 0.0 | 4.0 ± 0.0 | 4.0 ± 0.0 |
| Acetate | Residue | 1.7 ± 0.1 | 1.0 ± 0.0 | 1.0 ± 0.0 |
| Isopropanol | Extract | 4.0 ± 0.0 | 4.0 ± 0.0 | 4.0 ± 0.0 |
| | Residue | 2.2 ± 0.1 | 1.0 ± 0.0 | 1.0 ± 0.0 |
| Methanol | Extract | 4.0 ± 0.0 | 1.7 ± 0.0 | 1.0 ± 0.0 |
| | Residue | 1.8 ± 0.1 | 1.2 ± 0.1 | 1.0 ± 0.0 |
| Aliquots (15 ml each) of Pseudomonas fluorescens WH6 culture filtrate were taken to dryness in vacuo at 45° C. The dry solids recovered from a given aliquot were extracted three times (5 ml per extraction) with one of the indicated organic solvents. (Each extraction was performed by swirling the dry solids with the indicated volume of organic solvent for 5 minutes.) The three extracts prepared in this manner were combined, | | | | |
| # and the combined extracts were taken to dryness in vacuo at 45° C. The residual solids remaining after the extraction procedure were dried in a similar manner. For each original aliquot of the culture filtrate, the dried extract and the residue from the extraction procedure were dissolved in separate 15 ml aliquots of distilled water and tested for GAF activity in the Standard GAF Bioassay System (see Example 1) using seeds of annual bluegrass (Poa annua). | | | | |

*Germination Score of 1.0 = Germination completely arrested immediately after emergence of the coleorhiza and plumule. Germination Score of 4.0 = Normal germination and seedling development. (See Table 5 for details of the scoring system.)

TABLE 17
Solubility of WH6 GAF activity in aqueous ethanol solutions of varying
concentration

| | | Mean Germination Score* | | | |
|---|---|---|---|---|---|
| | | (±Standard Error of the Mean) | | | |
| | FRAC- | at relative sample concentration (X) indicated | | | |
| SOLVENT | TION | 0.03X | 0.1X | 0.3X | 1.0X |
| None | — | 2.0 ± 0.0 | 1.44 ± 0.0 | 1.0 ± 0.0 | 1.0 ± 0.0 |
| (Culture | | | | | |
| Filtrate) | | | | | |
| 95% Ethanol | Extract | 4.0 ± 0.0 | 4.0 ± 0.0 | 2.2 ± 0.2 | 1.2 ± 0.1 |
| | Residue | 2.6 ± 0.1 | 1.7 ± 0.1 | 1.0 ± 0.0 | 1.0 ± 0.0 |
| 90% Ethanol | Extract | 2.7 ± 0.1 | 2.0 ± 0.0 | 1.0 ± 0.0 | 1.0 ± 0.0 |
| | Residue | 3.8 ± 0.2 | 2.5 ± 0.1 | 1.0 ± 0.0 | 1.0 ± 0.0 |
| 85% Ethanol | Extract | 2.7 ± 0.1 | 1.6 ± 0.2 | 1.0 ± 0.0 | 1.0 ± 0.0 |
| | Residue | 4.0 ± 0.0 | 3.6 ± 0.2 | 2.0 ± 0.0 | 1.0 ± 0.0 |
| 75% Ethanol | Extract | 2.5 ± 0.1 | 1.5 ± 0.1 | 1.0 ± 0.0 | 1.0 ± 0.0 |
| | Residue | 4.0 ± 0.0 | 4.0 ± 0.0 | 4.0 ± 0.0 | 4.0 ± 0.0 |
| Aliquots (9 ml each) of Pseudomonas fluorescens WH6 culture filtrate were taken to dryness in vacuo at 45° C. The dry solids recovered from a given aliquot were extracted three times (3 ml per extraction) with the indicated concentration of aqueous ethanol. (Each extraction was performed by swirling the dry solids with the indicated volume of solvent for 5 minutes.) The three extracts prepared in this manner were combined, and the combined extracts were taken to dryness | | | | | |
| # in vacuo at 45° C. The residual solids remaining after the extraction procedure were dried in a similar manner. For each original aliquot of the culture filtrate, the dried extract and the residue from the extraction procedure were dissolved in separate 9 ml aliquots of distilled water and tested for GAF activity in the Standard GAF Bioassay System (see Example 1) using seeds of annual bluegrass (Poa annua). | | | | | |

*Germination Score of 1.0 = Germination completely arrested immediately after emergence of the coleorhiza and plumule. Germination Score of 4.0 = Normal germination and seedling development. (See Table 5 for complete details of the scoring system.)

TABLE 18
Evaluation of the Molecular Weight of GAF by Ultrafiltration

| | | RELATIVE | |
|---|---|---|---|
| ULTRAFILTRATION | | SAMPLE | GERMINATION |
| MEMBRANE | | CON- | SCORE* |
| (Molecular Weight | | CENTRATION | (±Standard |
| Cut-Off) | FRACTION | (X) | Error) |
| 3,000 | Retentate | 1.0X | 4.0 ± 0.0 |
| | | 0.3X | 4.0 ± 0.0 |
| | | 0.1X | 4.0 ± 0.0 |
| | Filtrate | 1.0X | 1.0 ± 0.0 |
| | | 0.3X | 2.5 ± 0.0 |
| | | 0.1X | 3.5 ± 0.2 |
| 1,000 | Retentate | 1.0X | 1.1 ± 0.1 |
| | | 0.3X | 2.5 ± 0.0 |
| | | 0.1X | 4.0 ± 0.0 |
| | Filtrate | 1.0X | 1.0 ± 0.0 |
| | | 0.3X | 2.5 ± 0.0 |
| | | 0.1X | 4.0 ± 0.0 |
| CONTROLS | Culture | 1.0X | 1.0 ± 0.0 |
| | Filtrate | 0.3X | 1.7 ± 0.2 |
| | | 0.1X | 2.5 ± 0.0 |
| | Water | — | 4.0 ± 0.0 |
| Aliquots (15 ml each) of Pseudomonas fluorescens WH6 culture filtrate were centrifuged (3000 × g, 2 hours per centrifugation) in Macrosep ® Centrifugal Concentrators (Pall Filtron Corp.) through ultrafiltration membranes of the indicated molecular weight cut-off values. The filtrate volumes were removed and brought to 15 ml with deionized water. The retentate volumes in the filtration devices | | | |
| # were made to 15 ml with deionized water and recentrifuged. The filtrates from this centrifugation were discarded, and the retentate volumes were recovered and made to 15 ml with deionized water. The filtrates and the washed retentates (from the second centrifugation) were then assayed for GAF activity in the Standard GAF Bioassay System (Example 1) over the range of concentrations indicated. | | | |

*Germination Score of 1.0 = germination completely arrested immediately after emergence of the coleorhiza and plumule. Germination Score of 4.0 = Normal germination and seedling development. (See Table 5 for details of the scoring system.)

TABLE 19
Heat stability of WH6 GAF activity

| | MEAN GERMINATION SCORE* (±SEM) | | | |
|---|---|---|---|---|
| HEAT | AT RELATIVE SAMPLE | | | |
| TREATMENT | CONCENTRATION (x) INDICATED | | | |
| (° C.) | 0.03X | 0.10X | 0.3X | 1.0X |
| None | 3.3 ± 0.2 | 1.8 ± 0.1 | 1.1 ± 0.0 | 1.0 ± 0.0 |
| (Room Temp) | | | | |
| 35 | 3.6 ± 0.2 | 1.7 ± 0.1 | 1.0 ± 0.0 | 1.0 ± 0.0 |
| 45 | 3.2 ± 0.3 | 1.6 ± 0.1 | 1.1 ± 0.0 | 1.0 ± 0.0 |
| 55 | 3.8 ± 0.1 | 1.8 ± 0.1 | 1.0 ± 0.0 | 1.0 ± 0.0 |
| 65 | 4.0 ± 0.0 | 1.9 ± 0.2 | 1.0 ± 0.0 | 1.0 ± 0.0 |
| 75 | 4.0 ± 0.0 | 3.9 ± 0.1 | 1.8 ± 0.2 | 1.2 ± 0.1 |
| 85 | 4.0 ± 0.0 | 4.0 ± 0.0 | 3.5 ± 0.0 | 3.0 ± 0.0 |
| 95 | 4.0 ± 0.0 | 4.0 ± 0.0 | 3.0 ± 0.0 | 2.6 ± 0.2 |
| 100 | 4.0 ± 0.0 | 4.0 ± 0.0 | 3.0 ± 0.0 | 2.9 ± 0.1 |
| (30 min) | | | | |
| Autoclave | 4.0 ± 0.0 | 4.0 ± 0.0 | 3.0 ± 0.0 | 2.9 ± 0.1 |
| (30 min) | | | | |

Aliquots (10 ml each) of Pseudomonas fluorescens WH6 culture filtrate were incubated at the indicated temperatures for a period of 1 hour, unless otherwise indicated. Each aliquot was then tested for GAF activity in the Standard GAF Bioassay System (see Example 1) using seeds of annual bluegrass (Poa annua).
*Germination Score of 1.0 = Germination completely arrested immediately after emergence of the coleorhiza and plumule. Germination Score of 4.0 = Normal germination and seedling development. (See Table 5 for details of the scoring system.)

TABLE 20
pH Stability of WH6 GAF activity

| | MEAN GERMINATION SCORE* | | | |
|---|---|---|---|---|
| | (±Standard Error of the Mean) | | | |
| | AT RELATIVE SAMPLE | | | |
| pH | CONCENTRATION (x) INDICATED | | | |
| TREATMENT | 0.03X | 0.10X | 0.3X | 1.0X |
| None | 2.3 ± 0.2 | 1.7 ± 0.1 | 1.0 ± 0.0 | 1.0 ± 0.0 |
| 2 | 2.7 ± 0.1 | 1.9 ± 0.1 | 1.1 ± 0.0 | 1.0 ± 0.0 |
| 4 | 3.0 ± 0.2 | 1.8 ± 0.2 | 1.1 ± 0.0 | 1.0 ± 0.0 |
| 6 | 2.5 ± 0.2 | 1.8 ± 0.1 | 1.0 ± 0.0 | 1.0 ± 0.0 |
| 8 | 2.5 ± 0.3 | 1.8 ± 0.1 | 1.0 ± 0.0 | 1.0 ± 0.0 |
| 10 | 2.3 ± 0.2 | 1.8 ± 0.1 | 1.0 ± 0.0 | 1.0 ± 0.0 |
| 12 | 2.5 ± 0.2 | 1.9 ± 0.2 | 1.0 ± 0.0 | 1.0 ± 0.0 |
| Aliquots (10 ml each) of Pseudomonas fluorescens WH6 culture filtrate were adjusted to the indicated pH values by addition of 1 N HCl or 1 N_NaOH. Each aliquot was held at the indicated pH value for 3 hours before readjusting to the starting pH. All aliquot samples were then tested for GAF | | | | |
| # activity in the Standard GAF Bioassay System (see Example 1) using seeds of annual bluegrass (Poa annua). | | | | |

*Germination Score of 1.0 = Germination completely arrested immediately after emergence of the coleorhiza and plumule. Germination Score of 4.0 = Normal germination and seedling development. (See Table 5 for details of the scoring system.)

TABLE 21
Test of the retention of WH6 GAF activity by Silica-C18

| | MEAN GERMINATION SCORE* | | | |
|---|---|---|---|---|
| | (±Standard Error of the Mean) | | | |
| | AT RELATIVE SAMPLE | | | |
| | CONCENTRATION (x) | | | |
| | INDICATED | | | |
| FRACTION | 0.1X | 0.25X | 0.5X | 1.0X |
| Sample Flow-Through | 4.0 ± 0.0 | 2.2 ± 0.3 | 1.3 ± 0.0 | 1.0 ± 0.0 |
| Buffer Wash | 4.0 ± 0.0 | 4.0 ± 0.0 | 4.0 ± 0.0 | 4.0 ± 0.0 |
| A sample (9 ml) of Pseudomonas fluorescens WH6 culture filtrate was passed through a Silica-C18 filtration device (Waters Sep-Pak VacRC Silica C18 Cartridge) equilibrated with 0.05 M KH₂PO₄ buffer (pH 6.5, KOH). The cartridge was then flushed with nine ml of the same buffer, and the | | | | |
| # sample flow-through and the buffer wash were assayed for GAF activity in the Standard GAF Bioassay System (see Methods) using seeds of annual bluegrass (Poa annua). | | | | |

*Germination Score of 1.0 = Germination completely arrested immediately after emergence of the coleorhiza and plumule. Germination Score of 4.0 = Normal germination and seedling development. (See Table 5 for details of the scoring system.)

It will be apparent that the precise details of the methods or compositions described may be varied or modified without departing from the spirit of the described disclosure. We claim all such modifications and variations that fall within the scope and spirit of the claims below.

1. An isolated bacterial strain selected from the group consisting of Pseudomonas fluorescens Biotype B E34, Pseudomonas fluorescens Biotype C WH19, Pseudomonas fluorescens Biotype C WH6, Pseudomonas putida Biotype B AH4, and Pseudomonas putida Biotype B AD31, wherein the bacterial strain inhibits or arrests grassy weed germination.

2.-5. (canceled)

6. A Germination-Arrest Factor, wherein the factor is produced by the isolated bacterial strain of claim 1, wherein the Germination-Arrest Factor inhibits or arrests grassy weed germination.

7. The Germination-Arrest Factor of claim 6, wherein the grassy weed is Poa annua (annual bluegrass), Poa trivialis (roughstalk bluegrass), or Bromus tectorum (downy brome), crabgrass, goosegrass, dallisgrass, bahiagrass, jointed goatgrass, rattail fescue, perennial ryegrass, or tall fescue.

8. (canceled)

9. The Germination-Arrest Factor of claim 6, wherein the Germination-Arrest Factor (a) is a hydrophilic molecule, (b) has a molecular weight less than 3,000 daltons, (c) reacts with ninhydrin, (d) comprises an ionizable group, or (e) a combination of two or more of (a), (b), (c), and (d).

10.-12. (canceled)

13. An isolated nucleic acid as set forth in:
(a) SEQ ID NO: 2;
(b) SEQ ID NO: 7;
(c) SEQ ID NO: 10; or
(d) sequences having at least 90% sequence identity to (a), (b), or (c);
wherein the nucleic acid encodes a Germination-Arrest Factor or a protein involved in the synthesis and or secretion of a Germination-Arrest Factor.

14. An isolated Germination-Arrest Factor protein encoded by the nucleic acid of claim 13 and comprising an amino acid sequence as set forth in:
(a) SEQ ID NO: 3;
(b) SEQ ID NO: 4;
(c) SEQ ID NO: 8;
(d) SEQ ID NO: 11;
(e) SEQ ID NO: 12;
(f) SEQ ID NO: 13;
(g) sequences having at least 90% sequence identity to (a), (b), (c), (d), (e), or (f); or
(h) conservative variants of (a), (b), (c), (d), (e), or (f);
wherein the Germination-Arrest Factor protein inhibits or arrests germination in grassy weeds or is involved in the synthesis or secretion of a Germination Arrest Factor.

15. The Germination-Arrest Factor protein of claim 14, wherein the grassy weed is Poa annua (annual bluegrass), Poa trivialis (roughstalk bluegrass), Bromus tectorum (downy brome), crabgrass, goosegrass, dallisgrass, bahiagrass, jointed goatgrass, rattail fescue, perennial ryegrass, or tall fescue.

16.-21. (canceled)

22. A method of inhibiting or arresting weed germination in a growth medium in which it would be desirable to inhibit or arrest grassy weed germination, or of inhibiting or arresting weed germination in grass seed, the method comprising applying a sample of the isolated bacterial strain of claim 1 or the Germination-Arrest Factor of claim 6 to the growth medium or to the grass seed in an amount sufficient to inhibit or arrest grassy weed germination.

23. (canceled)

24. The method of claim 22, wherein the sample or Germination-Arrest Factor is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide.

25. The method of claim 22, wherein the sample or Germination-Arrest Factor is applied in a formulation that is a solution, a soluble powder, an emulsifiable concentrate, a wettable powder, a liquid flowable, a dry flowable, a water-dispersible granule, a granule, or a pellet.

26.-29. (canceled)

30. A composition for inhibiting or arresting the germination of grassy weeds, comprising:
the Germination-Arrest Factor of claim 6; and
a timed- or temperature-release coating over at least a portion of the Germination-Arrest Factor.

31. The composition of claim 30, further comprising a water-resistant coating over the timed-or temperature-release coating.

32. A method of inhibiting or arresting weed germination in an area in which inhibiting or arresting weed germination is desirable, comprising:
broadcasting an herbicidally effective amount of the Germination-Arrest Factor of claim 6 at least once a year across the area, thereby inhibiting or arresting weed germination in the area.

33. The method of claim 32, wherein the area is a grass patch, an agricultural field, a natural landscape, or a road-side.

34. The method of claim 32, wherein the Germination-Arrest Factor is applied in a formulation that also comprises a surfactant, a stabilizer, a buffer, a preservative, an antioxidant, an extender, a solvent, an emulsifier, an invert emulsifier, a spreader, a sticker, a penetrant, a foaming agent, an anti-foaming agent, a thickener, a safener, a compatibility agent, a crop oil concentrate, a viscosity regulator, a binder, a tacker, a drift control agent, a fertilizer, an antibiotic, a fungicide, a nematicide, or a pesticide.

35. The method of claim 32, wherein the Germination-Arrest Factor is applied in a formulation that is a solution, a soluble powder, an emulsifiable concentrate, a wettable powder, a liquid flowable, a dry flowable, a water-dispersible granule, a granule, or a pellet.

36. The method of claim 35, wherein the Germination-Arrest Factor is formulated as a granule.

37. The method of claim 36, wherein the granule is at least partially coated with a timed-or temperature-release coating.

38. The method of claim 37, wherein the timed-or temperature-release coating is coated with a water-resistant coating.

39. The method of claim 32, wherein the method is a method of inhibiting grassy weeds among dicot species.

40. A method of producing the Germination-Arrest Factor of claim 6 comprising:
culturing Pseudomonas fluorescens Biotype B E34, Pseudomonas fluorescens Biotype C WH19, Pseudomonas fluorescens C Biotype WH6, Pseudomonas putida Biotype B AH4, Pseudomonas putida Biotype B AD31, or a combination thereof in a suitable culture medium;
collecting the culture medium; and
purifying the culture medium to produce the Germination-Arrest Factor.

41.-44. (canceled)

45. A kit for inhibiting or arresting grassy weed growth, comprising:
the Germination-Arrest Factor of claim 6; and
a container.

46.-47. (canceled)

48. A method of using the Germination-Arrest Factor of claim 6 to investigate regulation of seed germination and seedling development comprising using Germination-Arrest Factor to probe for regulatory sites in plant cells and regulatory mechanisms controlling seed germination and development.

49. A Pseudomonas fluorescens or Pseudomonas putida bacterial strain having the GAF-producing characteristics of Pseudomonas fluorescens Biotype B E34 (deposited as NRRL # B-30481), Pseudomonas fluorescens Biotype C WH19 (deposited as NRRL # B-30484), Pseudomonas fluorescens C Biotype WH6 (deposited as NRRL # B-30485), Pseudomonas putida Biotype B AH4 (deposited as NRRL # B-30482), or Pseudomonas putida Biotype B AD31 (deposited as NRRL # B-30483).

50. A Germination-Arrest Factor produced by the bacterial strain of claim 49, wherein Germination-Arrest Factor is a hydrophilic molecule, has a molecular weight less than 3,000 daltons, reacts with ninhydrin, and comprises an ionizable group.

51.-52. (canceled)
